SEQUENCE LISTING (12 sequences)

SEQ ID NO: 1 - protein, 272 aa, synthetic construct
  source 1..272: note = An exemplary RBD domain of a SARS-CoV-2 spike protein
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 2 - protein, 224 aa, synthetic construct
  source 1..224: note = an exemplary sequence used herein for the RBD domain corresponds to amino acids 319-541 of SARS-CoV-2 Spike
QRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 3 - protein, 598 aa, synthetic construct
  source 1..598: note = an exemplary sequence used herein for the ACE2 domain corresponds to amino acids 18-615 of the full-length human ACE2
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 4 - DNA, 2975 bp, synthetic construct
  source 1..2975: mol_type = other DNA; note = cDNA sequence encoding full-length Spike trimer protein
ggatccgccaccatgttcgtgtttctggtgctgctgcctctggtgtccagccagtgtgtgaacctgaccacaagaacccagctgcctcctgcgtacacaaacagcttcacccggggagtgtactaccccgataaggtgttccgtagctccgtgctgcactctacacaggacctgttcctcccctttttctctaacgtgacttggttccacgccatccacgtgagtggcaccaacggcaccaagagattcgacaatcctgttctgcccttcaacgacggcgtgtacttcgccagcacagagaagagcaacatcatcagaggatggatcttcggcaccactctcgatagcaagacccagtctctgctgatcgtcaacaatgccaccaatgtggtgatcaaggtttgtgaattccagttctgcaacgacccttttctgggcgtttactatcacaagaataacaagtcctggatggaaagcgagtttcgggtgtattcttctgccaacaactgtaccttcgagtacgtgtctcaaccttttctgatggacctggaaggcaagcagggcaacttcaaaaacctgagagaattcgtgttcaagaacattgatggctacttcaaaatctacagcaagcacacaccaatcaacctggtgagagacctgcctcagggcttcagcgccctggaacccctggtggacctgcctattggcattaacatcaccagattccagaccctgttggctctgcacagaagctacctgacacctggcgactccagcagcggatggaccgccggcgccgctgcttactacgtgggctacctgcagcctaggacattcctactgaagtacaatgagaacggcaccatcaccgatgccgtggattgcgccctggaccctctgagtgaaaccaagtgtaccctgaaatcgttcactgtcgagaagggcatctaccagaccagcaacttcagagtgcagcctacagagagcgcttcggtggcgtgtctgtgatcaccccgggtaccaacaccagcaaccaggtggctgttctttaccagggagtgaactgcaccgaagtgcccgtggccattcacgcagaccagctgacccccacctggcgggtgtactcaacaggcagcaatgtgttccagactcgggccggatgtctgatcggagctgaacacgtgaacaatagctacgagtgcgacatccccatcggcgctggcatctgcgcctcttaccagacccagaccaactccccaggatctgcctcttccgtggcctctcagagcatcatcgcctacaccatgagcctgggagccgagaatagcgtggcttacagcaacaactccatcgcgatccctacaaacttcaccatctctgtgaccaccgagatcctgccggtttctatgaccaagaccagcgttgattgcaccatgtacatctgcggcgattccacagagtgcagcaacctgctgctgcaatacggcagcttttgcacccagctcaacagagccctgaccggcatcgcagttgagcaggataagaacacacaggaggttttcgcccaagtgaaacaaatctacaagacccctcctatcaaggacttcggcgggtttaatttcagccaaatcctgcctgatccttctaaacccagcgcgggttctcccatcgaggacctgctgttcaacaaggtaacactcgctgacgccggcttcatcaagcagtatggcgactgcctgggcgatatcgctgccagagacctgatctgcgcccagaaattcaacggcctgacggtgctgcctcctctgctgaccgacgagatgatcgcccagtatacctctgccctcctggccggaacaatcaccagcggctggaccttcggcgccggacctgccctccagattcccttccctatgcagatggcctaccggttcaacggaatcggcgtcacccaaaacgtgctgtacgagaaccagaaactgatcgctaatcagttcaacagcgccatcggaaagatccaggacagcttgagtagcacaccaagcgccctgggcaagctgcaggatgtggttaatcagaacgcccaggccctgaacaccctggttaagcagttaagctctaactttggcgccatcagctccgtgctgaatgacatcctcagcagactggaccctcctgaggccgaggtgcagatcgaccggctgattacagggcggctgcaaagcctgcagacatacgtgacacagcaactaatccgggctgccgagatcagagcctccgccaacctggccgccacaaagatgtccgaatgcgtgctcggacagagcaagcgagtggacttctgcggaaagggctaccacctgatgagcttcccacagtccgccccccacggcgtcgtgttcctgcacgtgacatatgtgcctgcacaggaaaaaaacttcacaacagcccctgccatctgccacgacggcaaggcccacttccccagagagggcgtgttcgtgtccaacggaacacactggttcgtgacccaaagaaacttctacgagcctcagatcatcaccaccgataataccttcgtcagcggcaactgcgacgtggtgatcggcatcgtgaacaacacggtgtacgacccgttgcaaccagagttggatagctttaaggaagagctggacaagtacttcaagaatcacacctcccctgacgtggacctgggcgacatctccggcatcaacgccagcgtggtgaacatccagaaggaaatcgatagacttaacgaagtggccaagaacctgaacgagagcctgatcgaccttcaagagctgggcaaatacgagcagtacatcaagtggcctggcagcggttacatccctgaagcccctagagacggccaggcctatgtgcggaaagatggcgaatgggtcctgctgagcacgtttctgggacatcatcatcatcatcactaatgactcgag

SEQ ID NO: 5 - DNA, 3615 bp, synthetic construct
  source 1..3615: mol_type = other DNA; note = cDNA sequence encoding Loop-less Spike trimer protein
ggatccgccaccatgttcgtgtttctggtgctgctgcctctggtgtccagccagtgtgtgaacctgaccacaagaacccagctgcctcctgcgtacacaaacagcttcacccggggagtgtactaccccgataaggtgttccgtagctccgtgctgcactctacacaggacctgttcctcccctttttctctaacgtgacttggttccacgccatccacgtgagtggcaccaacggcaccaagagattcgacaatcctgttctgcccttcaacgacggcgtgtacttcgccagcacagagaagagcaacatcatcagaggatggatcttcggcaccactctcgatagcaagacccagtctctgctgatcgtcaacaatgccaccaatgtggtgatcaaggtttgtgaattccagttctgcaacgacccttttctgggcgtttactatcacaagaataacaagtcctggatggaaagcgagtttcgggtgtattcttctgccaacaactgtaccttcgagtacgtgtctcaaccttttctgatggacctggaaggcaagcagggcaacttcaaaaacctgagagaattcgtgttcaagaacattgatggctacttcaaaatctacagcaagcacacaccaatcaacctggtgagagacctgcctcagggcttcagcgccctggaacccctggtggacctgcctattggcattaacatcaccagattccagaccctgttggctctgcacagaagctacctgacacctggcgactccagcagcggatggaccgccggcgccgctgcttactacgtgggctacctgcagcctaggacattcctactgaagtacaatgagaacggcaccatcaccgatgccgtggattgcgccctggaccctctgagtgaaaccaagtgtaccctgaaatcgttcactgtcgagaagggcatctaccagaccagcaacttcagagtgcagcctacagagagcatcgtgcggttccctaacatcacaaatctgtgtcctttcggcgaggtgttcaacgccacaagatttgcctcagtgtacgcttggaataggaagagaatctccaactgtgtggccgactatagcgttctgtacaacagcgcgagcttcagcaccttcaagtgctacggcgttagccctaccaagctgaacgacctgtgcttcaccaacgtgtacgccgacagcttcgtcatcagaggagatgaggtgagacagatcgcccctggccagacaggcaaaatcgccgactacaactacaagctgcctgacgacttcactggctgcgtgatcgcctggaacagcaacaacctggacagcaaggtgggcggcacaaatggagtgggctaccagccctaccgcgtggtggtgctgagcttcgagctgctgcacgcccctgctaccgtgtgcggcccaaaaaagtctaccaacctggtgaagaataagtgcgtgaactttaacttcaacggcctgacaggaaccggcgtcctgaccgaaagcaacaaaaagttcctgccattccaacagtttggcagagatatcgctgacaccaccgacgccgtgcgggaccctcagaccctggaaatcctggacataacaccctgtagcttcggtggcgtgtctgtgatcaccccgggtaccaacaccagcaaccaggtggctgttctttaccagggagtgaactgcaccgaagtgcccgtggccattcacgcagaccagctgacccccacctggcgggtgtactcaacaggcagcaatgtgttccagactcgggccggatgtctgatcggagctgaacacgtgaacaatagctacgagtgcgacatccccatcggcgctggcatctgcgcctcttaccagacccagaccaactccccaggatctgcctcttccgtggcctctcagagcatcatcgcctacaccatgagcctgggagccgagaatagcgtggcttacagcaacaactccatcgcgatccctacaaacttcaccatctctgtgaccaccgagatcctgccggtttctatgaccaagaccagcgttgattgcaccatgtacatctgcggcgattccacagagtgcagcaacctgctgctgcaatacggcagcttttgcacccagctcaacagagccctgaccggcatcgcagttgagcaggataagaacacacaggaggttttcgcccaagtgaaacaaatctacaagacccctcctatcaaggacttcggcgggtttaatttcagccaaatcctgcctgatccttctaaacccagcgcgggttctcccatcgaggacctgctgttcaacaaggtaacactcgctgacgccggcttcatcaagcagtatggcgactgcctgggcgatatcgctgccagagacctgatctgcgcccagaaattcaacggcctgacggtgctgcctcctctgctgaccgacgagatgatcgcccagtatacctctgccctcctggccggaacaatcaccagcggctggaccttcggcgccggacctgccctccagattcccttccctatgcagatggcctaccggttcaacggaatcggcgtcacccaaaacgtgctgtacgagaaccagaaactgatcgctaatcagttcaacagcgccatcggaaagatccaggacagcttgagtagcacaccaagcgccctgggcaagctgcaggatgtggttaatcagaacgcccaggccctgaacaccctggttaagcagttaagctctaactttggcgccatcagctccgtgctgaatgacatcctcagcagactggaccctcctgaggccgaggtgcagatcgaccggctgattacagggcggctgcaaagcctgcagacatacgtgacacagcaactaatccgggctgccgagatcagagcctccgccaacctggccgccacaaagatgtccgaatgcgtgctcggacagagcaagcgagtggacttctgcggaaagggctaccacctgatgagcttcccacagtccgccccccacggcgtcgtgttcctgcacgtgacatatgtgcctgcacaggaaaaaaacttcacaacagcccctgccatctgccacgacggcaaggcccacttccccagagagggcgtgttcgtgtccaacggaacacactggttcgtgacccaaagaaacttctacgagcctcagatcatcaccaccgataataccttcgtcagcggcaactgcgacgtggtgatcggcatcgtgaacaacacggtgtacgacccgttgcaaccagagttggatagctttaaggaagagctggacaagtacttcaagaatcacacctcccctgacgtggacctgggcgacatctccggcatcaacgccagcgtggtgaacatccagaaggaaatcgatagacttaacgaagtggccaagaacctgaacgagagcctgatcgaccttcaagagctgggcaaatacgagcagtacatcaagtggcctggcagcggttacatccctgaagcccctagagacggccaggcctatgtgcggaaagatggcgaatgggtcctgctgagcacgtttctgggacatcatcatcatcatcactaatgactcgag

SEQ ID NO: 6 - DNA, 2967 bp, synthetic construct
  source 1..2967: mol_type = other DNA; note = cDNA sequence encoding RBD-less Spike trimer protein
ggatccgccaccatgttcgtgtttctggtgctgctgcctctggtgtccagccagtgtgtgaacctgaccacaagaacccagctgcctcctgcgtacacaaacagcttcacccggggagtgtactaccccgataaggtgttccgtagctccgtgctgcactctacacaggacctgttcctcccctttttctctaacgtgacttggttccacgccatccacgtgagtggcaccaacggcaccaagagattcgacaatcctgttctgcccttcaacgacggcgtgtacttcgccagcacagagaagagcaacatcatcagaggatggatcttcggcaccactctcgatagcaagacccagtctctgctgatcgtcaacaatgccaccaatgtggtgatcaaggtttgtgaattccagttctgcaacgacccttttctgggcgtttactatcacaagaataacaagtcctggatggaaagcgagtttcgggtgtattcttctgccaacaactgtaccttcgagtacgtgtctcaaccttttctgatggacctggaaggcaagcagggcaacttcaaaaacctgagagaattcgtgttcaagaacattgatggctacttcaaaatctacagcaagcacacaccaatcaacctggtgagagacctgcctcagggcttcagcgccctggaacccctggtggacctgcctattggcattaacatcaccagattccagaccctgttggctctgcacagaagctacctgacacctggcgactccagcagcggatggaccgccggcgccgctgcttactacgtgggctacctgcagcctaggacattcctactgaagtacaatgagaacggcaccatcaccgatgccgtggattgcgccctggaccctctgagtgaaaccaagtgtaccctgaaatcgttcactgtcgagaagggcatctaccagaccagcaacttcagagtgcaggctagcttcggtggcgtgtctgtgatcaccccgggtaccaacaccagcaaccaggtggctgttctttaccagggagtgaactgcaccgaagtgcccgtggccattcacgcagaccagctgacccccacctggcgggtgtactcaacaggcagcaatgtgttccagactcgggccggatgtctgatcggagctgaacacgtgaacaatagctacgagtgcgacatccccatcggcgctggcatctgcgcctcttaccagacccagaccaactccccaggatctgcctcttccgtggcctctcagagcatcatcgcctacaccatgagcctgggagccgagaatagcgtggcttacagcaacaactccatcgcgatccctacaaacttcaccatctctgtgaccaccgagatcctgccggtttctatgaccaagaccagcgttgattgcaccatgtacatctgcggcgattccacagagtgcagcaacctgctgctgcaatacggcagcttttgcacccagctcaacagagccctgaccggcatcgcagttgagcaggataagaacacacaggaggttttcgcccaagtgaaacaaatctacaagacccctcctatcaaggacttcggcgggtttaatttcagccaaatcctgcctgatccttctaaacccagcgcgggttctcccatcgaggacctgctgttcaacaaggtaacactcgctgacgccggcttcatcaagcagtatggcgactgcctgggcgatatcgctgccagagacctgatctgcgcccagaaattcaacggcctgacggtgctgcctcctctgctgaccgacgagatgatcgcccagtatacctctgccctcctggccggaacaatcaccagcggctggaccttcggcgccggacctgccctccagattcccttccctatgcagatggcctaccggttcaacggaatcggcgtcacccaaaacgtgctgtacgagaaccagaaactgatcgctaatcagttcaacagcgccatcggaaagatccaggacagcttgagtagcacaccaagcgccctgggcaagctgcaggatgtggttaatcagaacgcccaggccctgaacaccctggttaagcagttaagctctaactttggcgccatcagctccgtgctgaatgacatcctcagcagactggaccctcctgaggccgaggtgcagatcgaccggctgattacagggcggctgcaaagcctgcagacatacgtgacacagcaactaatccgggctgccgagatcagagcctccgccaacctggccgccacaaagatgtccgaatgcgtgctcggacagagcaagcgagtggacttctgcggaaagggctaccacctgatgagcttcccacagtccgccccccacggcgtcgtgttcctgcacgtgacatatgtgcctgcacaggaaaaaaacttcacaacagcccctgccatctgccacgacggcaaggcccacttccccagagagggcgtgttcgtgtccaacggaacacactggttcgtgacccaaagaaacttctacgagcctcagatcatcaccaccgataataccttcgtcagcggcaactgcgacgtggtgatcggcatcgtgaacaacacggtgtacgacccgttgcaaccagagttggatagctttaaggaagagctggacaagtacttcaagaatcacacctcccctgacgtggacctgggcgacatctccggcatcaacgccagcgtggtgaacatccagaaggaaatcgatagacttaacgaagtggccaagaacctgaacgagagcctgatcgaccttcaagagctgggcaaatacgagcagtacatcaagtggcctggcagcggttacatccctgaagcccctagagacggccaggcctatgtgcggaaagatggcgaatgggtcctgctgagcacgtttctgggacatcatcatcatcatcactaatgactcgag

SEQ ID NO: 7 - protein, 1249 aa, synthetic construct
  source 1..1249: note = Protein sequences of Full-length Spike trimer protein variant
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSAGSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGSGYIPEAPRDGQAYVRKDGEWVLLSTFLGHHHHHH

SEQ ID NO: 8 - protein, 1197 aa, synthetic construct
  source 1..1197: note = Protein sequences of Loop-less Spike trimer protein variant
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSAGSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGSGYIPEAPRDGQAYVRKDGEWVLLSTFLGHHHHHH

SEQ ID NO: 9 - protein, 981 aa, synthetic construct
  source 1..981: note = Protein sequences of RBD-less Spike trimer protein variant
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQASFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSAGSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGSGYIPEAPRDGQAYVRKDGEWVLLSTFLGHHHHHH

SEQ ID NO: 10 - protein, 1220 aa, synthetic construct
  source 1..1220: note = Protein sequences of the Full-length Spike monomer protein variant
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSAGSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGHHHHHH

SEQ ID NO: 11 - protein, 1168 aa, synthetic construct
  source 1..1168: note = Protein sequences of Loop-less Spike monomer protein variant
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSAGSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGHHHHHH

SEQ ID NO: 12 - protein, 952 aa, synthetic construct
  source 1..952: note = Protein sequences of RBD-less Spike monomer protein variant
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQASFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSAGSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGHHHHHH

(19) United States
(12) Patent Application Publication
(10) Pub. No.: US 2023/0251256 A1
(43) Pub. Date: Aug. 10, 2023

(54) MOLECULES, AND RELATED ASSAYS, TEST KITS AND METHODS

(71) Applicant: SAPPHIRE BIOTECH, INC., San Diego, CA (US)

(72) Inventors: Alim SEIT-NEBI, San Diego, CA (US); Sergei SVAROVSKY, San Diego, CA (US)

(21) Appl. No.: 18/296,328

(22) Filed: Apr. 5, 2023

Related U.S. Application Data
(63) Continuation-in-part of application No. PCT/US2022/077718, filed on Oct. 6, 2022.
(60) Provisional application No. 63/252,908, filed on Oct. 6, 2021.
(60) Provisional application No. 63/275,856, filed on Nov. 4, 2021.

(51) Int. Cl. G01N 33/543; G01N 33/569; G01N 21/78

(52) U.S. Cl. CPC G01N 33/54386; G01N 33/56983; G01N 21/78; G01N 2333/165; G01N 2469/20

(57) ABSTRACT

Molecules, test kits, test kit components and methods for detecting and measuring different first and second antibodies in a test sample using a single test are provided herein. A method includes the steps of obtaining the test specimen from a subject, transferring the test specimen to a sample receiving portion of an assay of a test kit, and reading the results from the assay. The test kit includes a first molecule comprising a first portion of a protein, wherein the first antibody has a first affinity to bind to the first portion, and a second molecule comprising a second portion of the protein different from the first portion, wherein the second antibody has a second affinity to bind to the second portion.

### CROSS-REFERENCE TO RELATED APPLICATIONS

This application is a Bypass Continuation-In-Part of International Patent Application No. PCT/US2022/077718, filed on Oct. 6, 2022, which is based on upon and claims priority to U.S. Provisional Patent Application Ser. No. 63/252,908, filed Oct. 6, 2021, and U.S. Provisional Patent Application Ser. No. 63/275,856, filed Nov. 4, 2021. These and all other extrinsic materials discussed herein, including publications, patent applications, and patents, are incorporated by reference in their entirety. Where a definition or use of a term in an incorporated reference is inconsistent or contrary to the definition of that term provided herein, the definition of that term provided herein applies and the definition of the term in the reference does not apply.

### SEQUENCE LISTING SUBMITTED ELECTRONICALLY

This application includes a sequence listing submitted electronically, in a file entitled 127607_0023 WO01_Sequence_Listing.xml, created Oct. 5, 2022 and having a file size of 31 KB, which is incorporated by reference herein.

In SEQ ID NOS. 4, 5, and 6, a part encoding signal peptide has the following sequence: ATGTTCGTGTTTCTGGTGCTGCTGCCTCTGGTGTCCAGC, where the bolded “AGT” is the protein translation initiation site. A part encoding the T4-phage fibritin trimerization domain has the following sequence: GGCAGCGGTTACATCCCTGAAGCCCCTAGAGACGGCCAGGCCTATGTGCGGAAAGATGGCGA ATGGGTCCTGCTGAGCACGTTTCTG. A part encoding the 6×His tag has the following sequence: CATCATCATCATCATCAC. The protein stop codon has the following sequence: TAATGA. Cloning restriction sites, 5′ BamHI and 3′ XhoI have the following sequences, respectively: GGATCC and CTCGAG.

In SEQ ID NOS. 7, 8, and 9, a Signal peptide has the following sequence: MFVFLVLLPLVSS. A T4-phage fibritin trimerization domain has the following sequence: GSGYIPEAPRDGQAYVRKDGEWVLLSTFL. A 6×Hs tag has the following sequence HHHHHH.

In SEQ ID NOS. 10, 11, and 12, a Signal peptide has the following sequence: MFVFLVLLPLVSS. A 6×Hs tag has the following sequence HHHHHH.

### TECHNICAL FIELD

This disclosure relates to proteins, and related assays, test kits, and methods.

### BACKGROUND

Due to their high specificity and selectivity antibodies have had the potential to be of use as biochemical tools for a range of applications including selection, identification, purification and as therapeutics. Broadly speaking antibodies are categorized into polyclonal antibodies and monoclonal antibodies. Antibodies can be utilized in research, diagnostics and therapeutics.

Antibodies are tools in many of the laboratory techniques. Due to their specificity they make tools that allow researchers to identify molecules that cannot be seen by the naked eye and thus enable conclusions to be drawn about the target molecule and pathway of interest.

Antibodies have become a component of many diagnostic assays. Uses included but are not limited to the detection of infections, recognition of allergies and the measurement of hormones and other biological markers in a biological sample such as blood.

### SUMMARY

In an aspect of the disclosure, a test kit for detection of a first antibody and a second antibody in a test specimen is provided, comprising a first molecule comprising a first portion of a protein, wherein the first antibody has a first affinity to bind to the first portion, and a second molecule comprising a second portion of the protein different from the first portion, wherein the second antibody has a second affinity to bind to the second portion. In some embodiments, the test kit further comprises a target molecule for the first molecule, for example, a target molecule the first molecule has an affinity to bind to.

In an aspect of the disclosure, a test kit for detection of functional and binding antibodies in a test specimen is provided, comprising a first molecule comprising an essential portion of a protein, a second molecule comprising a non-essential portion of the protein separate from the essential portion of the protein, and a target molecule for the essential portion of the protein, for example, a target molecule the essential portion of the protein has an affinity to bind to.

In some embodiments, such test kits can be, for example, utilized to detect and/or measure two (or more) different antibody types. In some embodiments, such test kits can be, for example, utilized to detect and/or measure amounts or concentration of two (or more) different antibody types in a single test (e.g., based on an intensity, optical density of a test line). Accordingly, in some embodiments, such test kits can be, for example, utilized to detect and/or measure and/or determine a quantitative relation (e.g., a ratio) between the two values to calculate or otherwise obtain a score (e.g., an immune response quality score, for example, an IC50 value or an EC50 (Effective Concentration) value) or titer. In some embodiments, a measurement or value associated with a first test line can be associated with an amount or concentration of a first antibody type (e.g., functional antibodies) in a sample. A measurement or value associated with a second test line can be associated with an amount or concentration of a second antibody type (e.g., binding antibodies) in a sample.

In an aspect of the disclosure, a method for detection of first and second antibodies in a test specimen is provided, comprising obtaining the test specimen from a subject, transferring the test specimen to a sample receiving portion of an assay of a test kit, and reading the results from the assay. In some embodiments, the test kit can further comprise a functional antibodies test line and a binding antibodies test line. In some embodiments, reading the results can comprise obtaining an indication indicating a level of immunity against a species or antigen, in an object (e.g., patient) taking a test using the test kit. In some embodiments, reading the results can comprise obtaining an indication indicating a level of immunity against a species or antigen, in an object (e.g., patient) taking the test, by relating, correlating, analyzing or considering two (or more) levels of two (or more) types of antibodies. For example, reading the results can comprise obtaining an immune response quality score based on a quantitative relation, such as a ratio, multiple, sum and other mathematical correlation, between a binding antibodies test line value and a functional antibodies test line value. For example, the functional antibodies test line value can be associated with an amount or concentration of functional antibodies in a sample. The binding antibodies test line value can be associated with an amount or concentration of binding antibodies in a sample. In some embodiments, obtaining the immune response quality score can comprise obtaining the binding antibodies test line value and the functional antibodies test line value, and determining a ratio between the binding antibodies test line value and the functional antibodies test line value (e.g., a ratio of the binding antibodies test line value to the functional antibodies test line value; a ratio of functional antibodies test line value to the binding antibodies test line value). In some embodiments, obtaining the immune response quality score can comprise comparing the ratio between the binding antibodies test line value and the functional antibodies test line value obtained from the test sample to known correlations between known test line values and associated an indication of immunity level or other associated values regarding an antigen or a target antigen, such as IC50 values, titer (or other immune response quality scores or score indicators). The test kit can further comprise a first molecule comprising a first portion of a protein, wherein the first antibodies have a first affinity to bind to the first portion, and a second molecule comprising a second portion of the protein different from the first portion, wherein the second antibodies have a second affinity to bind to the second portion. The test kit can further comprise a target molecule for the first molecule.

In yet another aspect of the disclosure, a protein variant is provided, the protein variant including an amino acid sequence selected from the group consisting of SEQ ID NOs: 8, 9, 11, 12.

Other advantages and benefits of the disclosed proteins, and related assays, test kits and methods will be apparent to one of ordinary skill with a review of the following drawings and detailed description.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic of a lateral flow assay (LFA) for detecting COVID-19 neutralizing antibodies;

FIG. 2 is a schematic of an immunoassay for detecting neutralizing antibodies and non-neutralizing antibodies, along with non-limiting examples of potential test results, according to an embodiment;

FIG. 3 are images of test results obtained using a test kit of the disclosure, according to an embodiment;

FIG. 4A is a schematic of a Spike protein trimer with an ACE2 receptor binding domain (or the “RBD”) including ACE2 binding loop (or “full length Spike protein trimer”) according to an embodiment;

FIG. 4B is a schematic of a Spike protein monomer with an ACE2 receptor binding domain including ACE2 binding loop (or “full length Spike protein monomer”) according to an embodiment;

FIG. 4C is a schematic of a Spike protein trimer with an ACE2 receptor binding domain not including an ACE2 binding loop (or “loop-less Spike protein trimer”) according to an embodiment;

FIG. 4D is a schematic of a Spike protein monomer with an ACE2 receptor binding domain not including ACE2 binding loop (or “loop-less Spike protein monomer”) according to an embodiment;

FIG. 4E is a schematic of a Spike protein trimer without an ACE2 receptor binding domain (or “RBD-less Spike protein trimer”) according to an embodiment;

FIG. 4F is a schematic of a Spike protein monomer without an ACE2 receptor binding domain (or “RBD-less Spike protein monomer”) according to an embodiment;

FIG. 5A is 4-20% reducing SDS-PAGE analysis of Spike protein variants expressed in HEK293F cells at 50 mL scale and purified by IMAC;

FIG. 5B is 4-20% reducing SDS-PAGE SDS-PAGE analysis of a loop-less Spike protein variants expressed in HEK293F cells at 50 mL scale and purified by IMAC;

FIG. 5C is 4-20% reducing SDS-PAGE SDS-PAGE analysis of a RBD-less protein variants expressed in HEK293F cells at 50 mL scale and purified by IMAC;

FIG. 6 is reducing and non-reducing 4-20% SDS-PAGE analysis of Spike protein variants expressed in HEK293F cells at 1 L scale and purified by IMAC;

FIG. 7 is a graph indicating evaluation of ACE2-binding activity of full-length, “RBD-less” and “Loop-less” Spike proteins by a direct ELISA.

FIG. 8 is a schematic of an immunoassay and molecules of a test kit, according to an embodiment;

FIG. 9 is a schematic of a mixed color immunoassay and molecules of a test kit, according to an embodiment;

FIG. 10 are images of exemplary test results obtained using the test kit of FIG. 6;

FIG. 11 illustrates a vertical flow assay, according to an embodiment; and

FIG. 12 illustrates a 96-channel assay plate for mass screening/surveillance, according to an embodiment.

FIG. 13 are images of test results obtained using a test kit of the disclosure, according to an embodiment;

FIG. 14 is a compilation of numeric data obtained from the test results of FIG. 13 using a lateral flow densitometer;

FIG. 15A illustrates a graph of the data from FIG. 14 using nAb alone and IC50;

FIG. 15B illustrates a graph of the data from FIG. 14 using bAb/nAb ratio and IC50;

FIG. 16 illustrates an example infrastructure, in which one or more of the processes described herein, may be implemented, according to an embodiment; and

FIG. 17 illustrates an example processing system, by which one or more of the processes described herein, may be executed, according to an embodiment.

### DETAILED DESCRIPTION

After reading this description it will become apparent to one skilled in the art how to implement the disclosed devices, components and methods in various alternative embodiments and alternative applications. However, all the various embodiments of the present disclosure will not be described herein. It is understood that the embodiments presented here are presented by way of an example only, and not limitation. As such, this detailed description of various alternative embodiments should not be construed to limit the scope or breadth of the present disclosure as set forth below.

Various recombinant proteins, test kits, test kit components and methods for detecting and measuring first and second different antibodies are provided herein. In some aspects, a test kit and method for detecting and measuring “binding antibodies” (for example, non-neutralizing antibodies) as well as “functional antibodies” (for example, neutralizing) in a single test and at the same time are provided. Such test kit and method can advantageously improve the diagnosis and therapy of various diseases.

As used herein, the term “binding antibodies” or “bAbs” refers to antibodies that bind to (or has an affinity to bind to) a protein without affecting its function of interest (as the “primary function”). As used herein, the term “functional antibodies” or “fAbs” refers to antibodies that affect the function of interest (as the “primary function”) of a protein upon binding. For example, in a given protein that has a functional portion of interest (as the “essential portion” or “essential part” of the protein or the “target portion” of the protein), a fAb will bind to (or has an affinity to bind to) part of the functional portion of interest of the given protein to affect the function of interest, and a bAb will bind to (or has an affinity to bind to) a different portion of the given protein, other than the functional portion of interest to affect the function of interest (as the “non-essential portion” or “non-essential part” of the protein). Functional antibodies may include, for example, neutralizing antibodies (nAbs), blocking antibodies (blAbs), and enhancing antibodies (eAbs). For example, if a protein is an enzyme, fAbs can, affect its activity by binding to a portion of the protein important for its catalytic activity and thus deactivating or enhancing enzymatic activity. Other antibodies that do not have affinities to the portion for its catalytic activity—for example, antibodies that bind only to portions of the protein unimportant for its catalytic activity—can be considered bAbs. If a protein is a cytokine, fAbs can, for example, bind to a portion of the protein involved in binding to a cytokine receptor, thereby preventing efficient cytokine-driven intracellular signaling. Other antibodies that do not have affinities to the portion of the protein involved in binding to a cytokine receptor—for example, that bind only to portions of the protein not involved in binding to a cytokine receptor—can be considered bAbs. If protein is a receptor, fAbs can, for example, bind to a portion of the protein essential for binding with its ligand (cytokine receptor, for example), thereby preventing efficient receptor-driven intracellular signaling. All other antibodies that do not have affinities to bind to the portion essential for the receptor—for example, that bind only to portions of the protein non-essential for binding with its ligand—can be considered bAbs.

In some embodiments, a component of a test kit for detecting and measuring fAbs and bAbs corresponding to a target protein of interest comprises a first protein having an essential part of the target protein of interest (with or without a non-essential part of the target protein) and a second protein having a non-essential part of the target protein of interest without having the essential part of the target protein. For example, the first and second proteins may be synthesized proteins, such as oligo- and poly-peptides or protein domains, or engineered proteins, that resemble essential and non-essential parts of the target protein of interest.

An aspect of the present disclosure is related to a test kit, a test kit component and a method for detecting and measuring a binding antibody (for example, a non-neutralizing antibody) as well as a functional antibody (for example, neutralizing) from the same bio sample added to the test kit for the test. Accordingly, the test kit measuring both binding and functional antibodies using the same bio sample can measure the two different antibody types in a single test, at the same time or using a single kit. In some embodiments, contemplated methods can comprise measuring the two different antibody types in a test sample in a single test and determining a quantitative relation between the two measured values (e.g., a ratio) and determine the quality or efficiency of the immune response based on known correlations between known ratios and IC50 values or other indicators of the quality or efficiency of the immune response. In some embodiments, the test kit can further comprise a functional antibodies test line and a binding antibodies test line. In some embodiments, reading the results can comprise obtaining an indication indicating a level of immunity against a species or antigen, in an object (e.g., patient) taking a test using the test kit. In some embodiments, reading the results can comprise obtaining an indication indicating a level of immunity against a species or antigen, in an object (e.g., patient) taking the test, by relating, correlating, analyzing or considering two (or more) levels of two (or more) types of antibodies. For example, reading the results can comprise obtaining an immune response quality score based on a quantitative relation, such as a ratio, multiple, sum and other mathematical correlation, between a binding antibodies test line value and a functional antibodies test line value. For example, the functional antibodies test line value can be associated with an amount or concentration of functional antibodies in a sample. The binding antibodies test line value can be associated with an amount or concentration of binding antibodies in a sample. In some embodiments, obtaining the immune response quality score can comprise obtaining the binding antibodies test line value and the functional antibodies test line value, and determining a ratio between the binding antibodies test line value and the functional antibodies test line value (e.g., a ratio of the binding antibodies test line value to the functional antibodies test line value; a ratio of functional antibodies test line value to the binding antibodies test line value). In some embodiments, obtaining the immune response quality score can comprise comparing the ratio between the binding antibodies test line value and the functional antibodies test line value obtained from the test sample to known correlations between known test line values and associated an indication of immunity level or other associated values regarding an antigen or a target antigen, such as IC50 values (or other immune response quality scores or score indicators). The test kit can further comprise a first molecule comprising a first portion of a protein, wherein the first antibodies have a first affinity to bind to the first portion, and a second molecule comprising a second portion of the protein different from the first portion, wherein the second antibodies have a second affinity to bind to the second portion. The test kit can further comprise a target molecule for the first molecule. Such test kit and method can increase the efficiency of the diagnosis and therapy of various diseases.

As an example, a test kit, test kit component and a method for detecting and measuring neutralizing and non-neutralizing antibodies to SARS-CoV-2 are provided herein.

SARS-CoV-2 is a 13 coronavirus and causes COVID-19, an acute respiratory infectious disease. Humans are generally susceptible. Individuals infected with SARS-CoV-2 are the main source of infection, including those who are asymptomatic. The main manifestations of COVID19 include fever, fatigue and dry cough. Nasal congestion, runny nose, sore throat, myalgia and diarrhea may also be present.

People who have recovered from COVID-19 have antibodies to the virus in their blood. Plasma prepared from these individuals is referred to as COVID19 convalescent plasma (CCP). CCP can be given to people with severe COVID-19 with the intention of boosting their ability to fight the virus.

Once someone recovers clinically and tests: (A) negative by PCR (no live virus present) and (B) positive by serology test (antibodies to SARS-Cov2 present), they may be asked if they would like to donate CCP. If they agree, they undergo plasmapheresis after which their plasma is then frozen, usually in 200 cc units.

When someone fighting COVID19 needs CCP, a unit of frozen plasma may be available. In order to determine whether the unit is suitable for use in someone fighting COVID19, there are certain assays available to detect neutralizing antibodies to SARS-CoV-2 as set forth in U.S. Patent Application Publication Nos. 2022/0205998, filed Feb. 1, 2022, 2022/0244258, filed on Dec. 16, 2021, and 2021/0356465, filed on May 12, 2021. Such assays detect a presence only of neutralizing antibodies (nAbs) against the essential part (e.g., receptor binding domain) of SARS-CoV-2 Spike proteins.

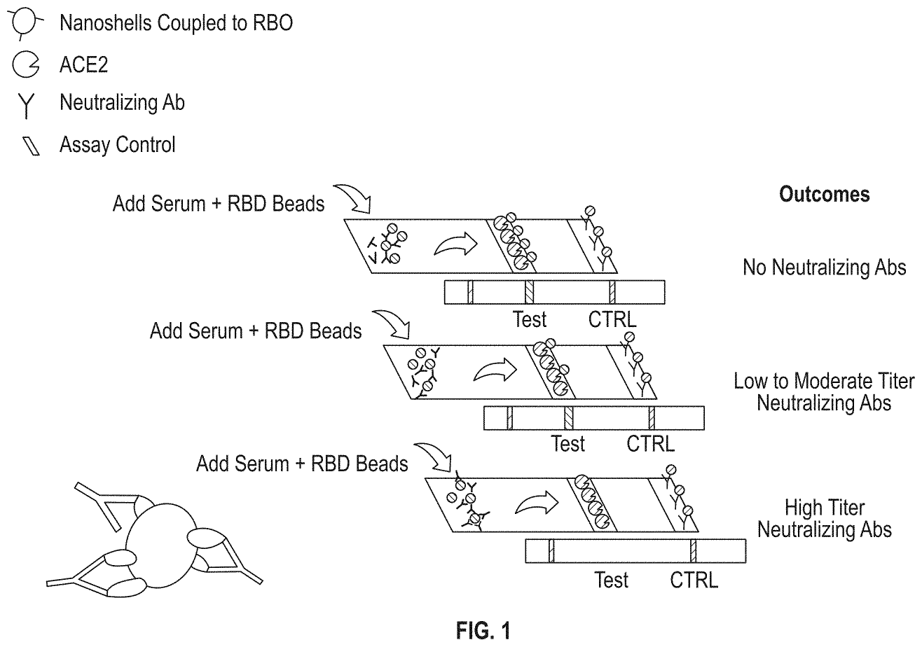

FIG. 1 is a schematic of a lateral flow assay (LFA) assay for detecting neutralizing antibodies to SARS-CoV-2. The assay is useful in detecting and measuring COVID-19 neutralizing antibody levels in a test sample, for example, in plasma or serum from individuals who have had recent or prior infection with SARS-CoV-2 or who have recovered from COVID19 or individuals who have received a COVID19 vaccine. The assay of FIG. 1 comprises a rapid test that utilizes a combination of SARS-COV-2 antigen coated colored particles (for example, nanoparticles coupled to RBD, nanoshells coupled to RBD) and ACE2 (for example, a modified human ACE2 protein receptor) for the detection of neutralizing antibodies to SARS-CoV-2 in serum or plasma that block interaction of the virus with human cells expressing ACE2.

The nanoparticle coupled to RBD can comprise a nanoparticle coupled to an essential part of a protein in relation to ACE2. As used herein, the phrase essential part of a protein in relation to ACE2 refers to any full length protein, functional fragment thereof (e.g., an RBD domain, an RBM and the like) that functions to bind to ACE2 (e.g., human ACE2) to facilitate gaining entry into cells to establish a coronavirus infection, e.g., a SARS-Cov-2 infection. Exemplary viral-ACE2 binding proteins are well-known in the art, and include Spike proteins (e.g., SARS CoV-2 Spike protein) or RBD domains thereof, and the like. In the case of coronaviruses, Spike proteins are large type I transmembrane protein trimers that protrude from the surface of coronavirus virions. Each Spike protein comprises a large ectodomain (comprising S1 and S2), a transmembrane anchor, and a short intracellular tail. The S1 subunit of the ectodomain mediates binding of the virion to host cell-surface receptors through its receptor-binding domain (RBD). The S2 subunit fuses with both host and viral membranes, by undergoing structural changes.

SARS-CoV-2 utilizes the Spike glycoprotein to interact with cellular receptor ACE2 (Zhou et al., Nature 579: 270-273, doi:10.1038/s41586-020-2012-7 (2020); Hoffmann et al., Cell, S0092-8674(0020)30229-30224, doi:10.1016/j.ce11.2020.02.052 (2020) doi:10.1016/j.ce11.2020.02.052 (2020). The amino acid sequence of the SARS-CoV-2 Spike protein has been deposited with the National Center for Biotechnology Information (NCBI) under Accession No. QHD43416. Binding with ACE2 triggers a cascade of cell membrane fusion events for viral entry. The high-resolution structure of SARSCoV-2 RBD bound to the N-terminal peptidase domain of ACE2 has recently been determined, and the overall ACE2-binding mechanism is virtually the same between SARS-CoV-2 and SARS-CoV RBDs, indicating convergent ACE2-binding evolution between these two viruses (Gui et al., CellRes 27, 119-129, doi:10.1038/cr.2016.152 (2017); Song et al., PLoS Pathog 14, e1007236-e1007236, doi:10.1371/journal.ppat.1007236 (2018); Yuan et al., Nat Commun 8, 15092-15092, doi:10.1038/ncomms15092 (2017); and Wan et al., J Virol,

JVI.00127-00120, doi:10.1128/JVI.00127-20 (2020)). This suggests that disruption of the RBD and ACE2 interaction, e.g., by neutralizing antibodies, would block SARS-CoV-2 entry into the target cell. Indeed, a few such disruptive agents targeted to ACE2 have been shown to inhibit SARS-CoV infection (Kruse, R. L., F1000Res, 9: 72-72; doi:10.12688/f1000research.22211.2 (2020); and Li et al., Nature 426, 450-454; doi: 10.1038/nature02145 (2003)). In addition, neutralizing antibodies directed against coronaviruses (also referred to herein as “coronavirus neutralizing antibodies”) have been identified and isolated (see, e.g., Liu et al., Potent neutralizing antibodies directed to multiple epitopes on SARS-CoV-2 Spike. Nature (2020). doi.org/10.1038/s41586-020-2571-7; Rogers et al., Science 15 Jun. 2020:eabc7520; DOI: 10.1126/science.abc7520; Alsoussi et al., J Immunol Jun. 26, 2020, ji2000583; DOI: /doi.org/10.4049/jimmuno1.2000583; Kreer et al., Cell, S0092-8674(20)30821-7. 13 Jul. 2020, doi:10.1016/j.cell.2020.06.044; Tai et al., J Virol. 2017 Jan. 1; 91(1): e01651-16; and Niu et al., J Infect Dis. 2018 Oct. 15; 218(8): 1249-1260).

The peptide comprising a receptor binding domain (RBD) of a coronavirus Spike protein may be prepared using routine molecular biology techniques, such as those disclosed herein. The nucleic acid and amino acid sequences of RBDs of various coronavirus Spike proteins are known in the art (see, e.g., Tai et al., Cell Mol Immunol 17, 613-620 (2020). doi.org/10.1038/s41423-020-0400-4; and Chakraborti et al., Virology Journal volume 2, Article number: 73 (2005); and Chen et al., Biochemical and Biophysical Research Communications, 525(1): 135-140 (2020)). An exemplary RBD domain of a SARS-CoV-2 Spike protein comprises the amino acid sequence SEQ ID NO: 1.

In other particular embodiments, an exemplary sequence used herein for the RBD domain corresponds to amino acids 319-541 of SARS-CoV-2 Spike, set forth as SEQ ID NO: 2. Those skilled in the art will recognize that functional fragments of SEQ ID NO: 1 and/or SEQ ID NO: 2 can also be used in the invention methods and devices.

In particular embodiments, an exemplary sequence used herein for the ACE2 domain corresponds to amino acids 18-615 of the full-length human ACE2, set forth as SEQ ID NO:3.

Those skilled in the art will recognize that functional fragments of SEQ ID NO:3 can also be used in the invention methods and devices.

The assay of FIG. 1 can be used to measure levels of neutralizing antibodies against Spike protein receptor binding domains (RBD) that block the RBDs from binding to ACE2 receptors. The addition of serum or plasma lacking nAbs (top) does not block binding of RBD-beads to ACE2 resulting in the RBD-bead—ACE2 complex creating a visible line at the test location (e.g., test line). The addition of moderate titer nAbs to the sample pad creates a weak line at the test location (middle). The addition of high titer nAbs (>1:640) blocks binding of RBD-beads to ACE2 such that no line is observed at the test location on the strip (bottom). The control location (e.g., control line) downstream of the test line represents capture of gold nanospheres coupled to a monoclonal antibody (e.g., a mouse Mab, or the like).

In one embodiment, a test uses immobilized polystreptavidin (test line TEST) and goat anti-mouse IgG (control line CTRL) on a nitrocellulose strip. In an embodiment, the conjugate pad contains recombinant SARS-CoV-2 antigen (Spike protein RBD domain from SARS-CoV-2) conjugated with dark green gold Nanoshells and a mouse antibody conjugated to red gold Nanospheres. The sample pad contains tagged (e.g., biotinylated) human ACE2 protein. During testing, in a particular embodiment, anti-RBD antibodies in plasma or serum bind to RBD-conjugated dark green gold Nanoshells in the test cassette. When assay (chase) buffer is added to the sample well, the dried components on the strip interact with plasma or serum from whole blood. If the sample contains antibodies that prevent RBD from binding to ACE2 (neutralizing antibodies), the test will show a light or ghost Test line. If the sample does not contain, or contains low levels of neutralizing antibodies, RBD-gold Nanoshells and ACE2-biotin will interact forming a dark green Test line.

The results described above are for the semi-quantitative measurement of only antibodies that neutralize SARS-CoV-2. However, as described herein, there is a need for the detection of functional (e.g., neutralizing) and binding (e.g., non-neutralizing) antibodies in a single test. There is also a need for an immunoassay for measuring or identifying a ratio of functional antibodies to binding antibodies, binding antibodies to functional antibodies, functional antibodies to total antibodies, and/or binding antibodies to total antibodies. Such ratio can be associated with an indicator indicating an immunity level of interest or compared to correlations between the indicator indicating an immunity level of interest and a level(s) of one, two, three or more types of antibodies. For example, known IC50 values may be based on to determine an immune response quality score. The immune response quality score can be, among other things, an IC50 value, an IC50 value range, or a grade or score based thereon. IC50, the half maximal inhibitory concentration, is a measure of potency of an antibody or any other substance inhibiting a specific biological function, such as virus particle binding to human cells.

An aspect of the disclosure is providing a test kit for detecting both neutralizing and non-neutralizing antibodies.

In some embodiments, a test kit and method for detecting and measuring neutralizing and non-neutralizing antibodies to SARS-CoV-19 are provided. In this case, for example, an antibody that disrupts RBD-ACE2 interaction is considered a functional and neutralizing antibody (fAb and nAb) and an antibody that does not disrupt RBD-ACE2 interaction is considered a binding antibody and non-neutralizing antibody (bAb and non-nAb).

An aspect of the disclosure is related to engineered Spike proteins.

For example, in order to measure non-nAbs on the same strip as nAbs, the inventors engineered novel Spike proteins devoid of any ACE2 binding activity. Exemplary engineered Spike proteins include a RBD-less Spike protein trimer or monomer (Spike protein completely missing RBD domain) a loop-less Spike protein trimer or monomer (Spike protein missing only ACE2-binding motif such as a RBD binding loop, in the RBD domain, while maintaining other portion of RBD such as a non-ACE2 binding RBD epitope).

In some embodiments, a biological sample as the test-specimen or test sample is whole blood, plasma or serum. In some embodiments, the whole blood, plasma or serum is obtained from a person either known or suspected of recovering from an infectious (e.g., COVID-19, HIV, Influenza, Hepatitis B, Hepatitis C, Zika, Adenovirus) disease, or known to have been vaccinated for a virus that causes the infectious disease (e.g., SARS-CoV-2, human immunodeficiency virus, human influenza A virus, human influenza B virus, hepatitis B virus, hepatitis C virus, Zika virus). In some embodiments, the plasma is obtained using anti-coagulants such as heparin, dipotassium EDTA or sodium citrate, and the like.

In some embodiments, the test-specimen or test sample is whole blood, plasma, serum and/or saliva. In some embodiments, the whole blood, plasma, serum or saliva is obtained from a person either known or suspected of recovering from an infectious (e.g., COVID-19) disease, or known to have been vaccinated for a virus that causes the infectious disease (e.g., SARS-CoV-2).

While certain assays available to detect neutralizing antibodies to SARS-CoV-2 are known, non-neutralizing antibodies may also be important in providing protection against COVID-19. If nAb levels are low, test results from known assays can be inconclusive, and an individual may be left wondering if they have any immune response at all.

While some examples herein relates to a test kit and method for detecting and measuring nAbs and non-nAbs in a bio sample in a single test, it should be appreciated that the molecules, test kits, test kit components and methods disclosed herein can be used to detect and measure any first and second antibodies, for example, functional and binding antibodies, in a single test as further described in detail herein. As noted above, binding antibodies bind to a protein without affecting its primary or essential function, while functional antibodies affect the essential or primary function of a protein upon binding. For example, it should be appreciated that the assays, test kits, methods and molecules described herein can be suitable for detecting and measuring other functional and binding antibodies, and determining an indication of an immunity level against an antigen, such as an IC50 value, or other immune response quality score based on an association or correlation (e.g., a ratio) among different types of antibodies and/or an antigen, such as antigen to an antibody, functional antibodies to binding antibodies, binding antibodies to functional antibodies, functional antibodies to total antibodies, and/or binding antibodies to total antibodies. Such determination can also be based on, for example, known correlations between known ratios and, for example, known IC50 values.

In an aspect of the disclosure, a test kit for detection of a first antibody and a second antibody in a test specimen is provided, comprising a first molecule comprising a first portion of a protein, wherein the first antibody has an affinity to bind to the first portion, a second molecule comprising a second portion of the protein different from the first portion, wherein the second antibody has an affinity to bind to the second portion, and a target molecule for the first molecule (e.g., a target molecule that the first molecule has an affinity to bind to).

The test kit can further comprise an immunoassay having a detection zone, the detection zone comprising at least one test location, and wherein the at least one test location comprises a first anti-tag. The detection zone can comprise a nitrocellulose membrane.

In some embodiments, the at least one test location of the detection zone comprises two test locations, the first anti-tag bound to the first test location, and a second anti-tag bound to the second test location. The second anti-tag can be different from the first anti-tag. The target molecule, for example, a domain or a fragment, such as a functional domain or polypeptide fragment, such as a fragment of cellular receptor, can be bound to a first tag, and the first tag/first anti-tag can form a first tag/anti-tag pair. In some embodiments, a variety of domains or fragments can be implemented. For examples, contemplated functional domains or polypeptide fragments of include ACE2 for SARS-CoV-2, CD4 for human immunodeficiency virus, sodium taurocholate co-transporting polypeptide (NTCP) for Hepatitis B, AXL receptor tyrosine kinas for Zika virus, CD81 for Hepatitis C, or CAR and CD46 for Adenovirus. The first molecule can be coupled to a first label, and a coupling molecule can be bound to a second label. The coupling molecule can, similar to the second molecule, comprise the second portion of the protein. The second molecule can be bound to a second tag, and the second tag/second anti-tag can form a second tag/anti-tag pair.

In some other embodiments, the at least one test location comprises a single test location, and the first anti-tag is bound to the single test location. The target molecule can bound to a first tag, and the first tag/first anti-tag can form a first tag/anti-tag pair. The first molecule can be coupled to a first label, the second molecule can be bound to a second tag (which can be the same as the first tag), and a coupling molecule can be bound to a second label. In some embodiments, the coupling molecule comprising the second portion of the protein. The second tag (which can be the same as the first tag) and the first anti-tag can form a tag/anti-tag pair.

The immunoassay can also include a sample receiving portion and a conjugate release pad. The sample receiving portion can comprise at least one of a sample port, a sample pad, and a sample filter.

In some embodiments, the first antibody is a functional antibody, the second antibody is a binding antibody, the first portion of the protein comprises an “essential portion” of the protein, and the second portion comprises a “non-essential portion” of the protein, as described above.

In some embodiments, the protein is a viral-ACE2-binding protein (e.g., a Spike protein), the first portion comprises an ACE2-binding motif of a receptor binding domain (RBD), the first antibody is a neutralizing antibody (NAb), the second portion lacks the ACE2-binding motif of the RBD, the second antibody is a non-neutralizing antibody (nNAb), and the target molecule for the first molecule comprises ACE2 or a functional fragment thereof.

In another aspect of the disclosure, a test kit for the detection of functional and binding antibodies in a test specimen is provided, comprising a first molecule comprising an “essential portion” of a protein, a second molecule comprising a “non-essential portion” of the protein separate from the essential portion of the protein, and a target molecule for the essential portion of the protein.

The test kit can further comprise an immunoassay having a detection zone, the detection zone comprising at least one test location, and wherein the at least one test location comprises a first anti-tag. The detection zone can comprise a nitrocellulose membrane.

In some embodiments, the at least one test location of the detection zone comprises a functional antibodies test region comprising the first anti-tag, and binding antibodies test location comprising a second anti-tag. The second anti-tag can be different from the first anti-tag. The target molecule can be bound to a first tag, and the first tag/first anti-tag can form a first tag/anti-tag pair. The first molecule can be coupled to a first label, and a coupling molecule can be bound to a second label. The coupling molecule can, similarly to the second molecule, comprise the second portion of the protein. The second molecule can be bound to a second tag, and the second tag/second anti-tag can form a second tag/anti-tag pair.

In some other embodiments, the at least one test location comprises a single test location, and the first anti-tag is bound to the single test location. The target molecule can bound to a first tag, and the first tag/first anti-tag can form a first tag/anti-tag pair. The first molecule can be coupled to a first label, the second molecule can be bound to a second tag (which can be the same as the first tag), and a coupling molecule can be bound to a second label. In some embodiments, the coupling molecule comprising the second portion of the protein. The second tag (which can be the same as the first tag) and the first anti-tag can form a tag/anti-tag pair.

In some embodiments, the protein is a viral-ACE2-binding protein, the essential portion comprises an ACE2-binding motif of a receptor binding domain (RBD), the functional antibody is a neutralizing antibody (NAb), the non-essential portion lacks the ACE2-binding motif of the RBD, the binding antibody is a non-neutralizing antibody (nNAb), and the target molecule for the first molecule comprises ACE2 or a functional fragment thereof.

In some embodiments, the essential portion and the non-essential portion each comprise engineered portions of the protein.

In another aspect of the disclosure, a method for detection of first and second antibodies (e.g., functional and binding antibodies) in a test specimen is provided, comprising obtaining the test specimen from a subject, and transferring the test specimen to a sample receiving portion of an assay of a test kit. The test kit can further comprise: a first molecule comprising a first portion of a protein (e.g., an essential part of the protein), wherein the first antibodies have an affinity to bind to the first portion; a second molecule comprising a second portion of the protein (e.g., a non-essential part of the protein) different from the first portion, wherein the second antibodies have an affinity to bind to the second portion; and a target molecule for the first molecule (e.g., ACE2 or fragment thereof). The method can further comprise the step of transferring the first molecule, the second molecule, and the target molecule to the assay. Additionally or alternatively, the first molecule, the second molecule and/or the target molecule may already be held on the assay, for example, on the conjugate release pad. The method can further comprise the step of adding a buffer. When assay (chase) buffer is added to the sample well, the dried components on the strip interact with plasma or serum from whole blood. In some embodiments, the method can further comprise the step of reading the results from the assay. In some embodiments, the assay comprises a detection zone at least one test location, and the at least one test location comprises a first anti-tag.

In some embodiments, the protein is a viral-ACE2-binding protein, the first portion comprises an ACE2-binding domain of the viral-ACE2-binding protein, the second portion lacks the ACE2-binding domain of the viral-ACE2-binding protein, and the target molecule is ACE2 of a functional fragment thereof.

In some embodiments, the detection zone comprises a single test location comprising a first anti-tag, wherein the target molecule is bound to a first tag, wherein the first molecule is coupled to a first label, wherein the second molecule is coupled to a second tag, and further comprising a coupling molecule coupled to a second label, the coupling molecule comprising the second portion of the protein. In some embodiments, the detection zone comprises a first antibodies test location comprising the first anti-tag, a second antibodies test location comprising a second anti-tag, wherein the target molecule is bound to a first tag, wherein the first molecule is coupled to a first label, wherein the second molecule is bound to a second tag, and further comprising a coupling molecule coupled to a second label, the coupling molecule comprising the second portion of the protein. In some embodiments, the target molecule is bound to biotin, and wherein the first anti-tag comprises streptavidin. In some embodiments, each of the first label and the second label is selected from a nanoparticle, bead, latex bead, aptamer, oligonucleotide, a quantum dot, and a combination thereof. In some embodiments, the first molecule is coupled to a gold nanoshell (GNS), and wherein the coupling molecule is coupled to a gold nanosphere (GNP). In some embodiments, the method further comprises determining a ratio of the first antibodies to the second antibodies and/or % measurement of the first and second antibody content using a color deconvolution algorithm. In some embodiments, a color microelectronic chip (color chip) records a color on a test line and splits it up into, for example, Red (R), Green (G) and Blue (B) components in a digitized form. If, for example, Red and Blue beads are used to construct the assay, the amount of R and B beads can be readily determined. Dividing R over B can provide a ratiometric measurement of different antibodies (e.g., binding and functional, neutralizing and non-neutralizing). In some embodiments, the method further comprises determining a ratio of the first antibodies to the second antibodies based on a color appearing on the test region. In some embodiments, determining the ratio comprises densitometry detection/determining an intensity of each of the test lines (e.g., functional antibodies test line, binding antibodies test line) using a densitometer or other device, and calculating or otherwise obtaining the ratio. In some embodiments, the method further comprises determining an immune response quality score based at least in part on the ratio and a comparison of the ratio to other known ratios and associated IC50 values.

As set forth herein, several embodiments of the present invention include lateral and vertical flow detection test-cassette devices and systems for detecting and/or quantifying a particular target analytes based on detecting complex formation of the analytes with known receptors in either colorimetric, fluorometric, radiometric, magnetometric, or thermometric detector modes. Other biochemical assays using the same principle include Enzyme Linked Immunoassay (ELISA), AlphaLisa, chemiluminescent, elecrochemiluminescent (e.g. Mesoscale Devices), multiplexed bead-based assays (e.g. Luminex), radioimmunoassays (RIA), electrochemical impedance spectroscopy (EIS), amperometric, voltametric, field effect transistor (FET) based bioassays, as well as other methods capable of quantifying immunoassay responses, all of which are contemplated for the molecules, assays, test kits, and methods described herein.

In some embodiments, assay systems, test-cassette devices and methods of the present invention, include an analytical membrane that is divided into one or more detection/test regions and optionally one or more control regions. The detection region or regions can include a target analyte binding agent immobilized to a portion of the detection region such that it is not displaced when facilitating lateral flow across the analytical membrane. Lateral and/or vertical flow assay systems of the present disclosure can also include a sample receiving portion and/or conjugate pad within which a target analyte binding agents are contained. In some embodiments, a target analyte binding agents are contained within the conjugate pad but flows from the conjugate pad and across the analytical membrane (also referred to herein as detection zone) towards the detection and control regions when lateral flow occurs. Lateral and/or vertical flow assay systems of the present disclosure can also include a sample pad that is positioned at one distal end of the lateral flow assay system (e.g., opposite an absorbent pad).

In some embodiments, a plasma test sample is collected using a tube containing Heparin, EDTA and/or ACD anti-coagulants. In certain embodiments, the serum or plasma is separated from blood as soon as possible to avoid hemolysis. In some embodiments, testing should be performed immediately after specimen collection unless immediately frozen below −20° C. Specimens should not be left at room temperature for longer than 3 days. Serum and plasma specimens may be stored at 2-8° C. for up to 3 days. For long-term storage, specimens should be kept below −20° C. Specimens should be brought to room temperature prior to testing. Frozen specimens should be completely thawed and mixed well prior to testing. Specimens should not be frozen and thawed more than once. If specimens are to be shipped, they should be packed in compliance with federal regulations for transportation of etiologic agents.

In some embodiments, the test sample comprises whole blood and the sample receiving portion includes a blood filter placed on top of the conjugate pad to receive the whole blood sample for analysis. In one embodiment, the blood filter functions to exclude at least red blood cells (and the like) from proceeding with the filtrate into the reaction portion of the assay. In a particular embodiment, the filtrate is serum that is then used for the analysis for the presence, absence and/or quantity of binding and functional antibodies therein.

A sample that contains (or may contain) a target analyte (e.g., functional antibodies, binding antibodies) is applied to the sample pad. In some embodiments, a lateral flow assay system also comprises a wicking pad at an end of the device distal to the sample pad. The wicking pad generates capillary flow of the sample from the sample pad through the conjugate pad, analytical membrane, detection region, and control region.

In accordance with some embodiments, upon addition of a test-specimen to the sample pad, the facilitation of flow (e.g., lateral flow) causes a target-analytes within the sample, if any, to contact a target analyte binding agents within the conjugate pad; subsequently, flow (e.g., lateral flow) causes the target analytes and the target analyte binding agents to contact a second target analyte binding agent immobilized to a detection region of the analytical membrane. The presence and/or quantity of the target analytes is then determined based on detection of the analytes in the detection regions also referred to herein as a “test line” or “test region” and/or in comparison to the control.

In some embodiments, levels of functional and binding antibodies can be interpreted by comparing the intensity of a test line in the cassette with a supplied scorecard that is color-matched to actual test lines. In some embodiments, levels of functional and binding antibodies can be interpreted using a densitometer or device that quantifies an intensity, optical density and/or other value associated with a characteristic of the test region/line. In some embodiments, the test lines develop within ten (10) minutes when the test is properly performed. Reading test results earlier than 10 minutes (or after 20 minutes) after the addition of a buffer may yield erroneous results. Repeat testing should be performed if the control line does not develop. Repeat testing should also be performed if the user is unsure he/she performed the test according to the instructions.

Table 1 is a non-limiting example of how
certain test results can be interpreted.

| | | Functional | Binding | |
|---|---|---|---|---|
| | | Antibodies | Antibodies | |
| | C Line | Test Line | Test Line | Test Result Interpretation |
| 1 | not | Any | Any | Invalid Test. The specimen |
| | present | | | must be retested with another |
| | | | | cassette |
| 2 | + | No or very | Very | Valid Test, High levels of |
| | | faint line | faint line | functional antibodies and |
| | | | | low levels of binding |
| | | | | antibodies present. Compare |
| | | | | to scorecard. |
| 3 | + | Moderately | Moderately | Valid Test, Moderate levels |
| | | positive Line | positive line | of functional antibodies and |
| | | | | binding antibodies present. |
| | | | | Compare to scorecard |
| 4 | + | Strongly | Strong | Valid Test, Low level or no |
| | | positive line | positive line | functional antibodies and |
| | | | | high levels of binding |
| | | | | antibodies present. Compare |
| | | | | to scorecard |
| 5 | + | No or very | Strong | Valid Test, High levels |
| | | faint line | positive line | of functional antibodies |
| | | | | and high levels of binding |
| | | | | antibodies present. Compare |
| | | | | to scorecard. |
| 6 | + | Moderately | Very | Valid Test, Moderate levels |
| | | positive Line | faint line | of functional antibodies and |
| | | | | low levels of binding |
| | | | | antibodies present. Compare |
| | | | | to scorecard |

In some embodiments, in addition to the scorecard (or alternatively), a lateral flow reader or a scanner can be used to quantitate the lines. In some embodiments, the test kit should be stored at room temperature prior to use. In some embodiments, some or all of the directions for use, storage recommendations, and/or specimen collection and preparation steps related to IMMUNOPASS test kits, for example, those described in U.S. Patent Application Publication No. 2022-0205998, can be utilized for test kits and methods of the disclosure.

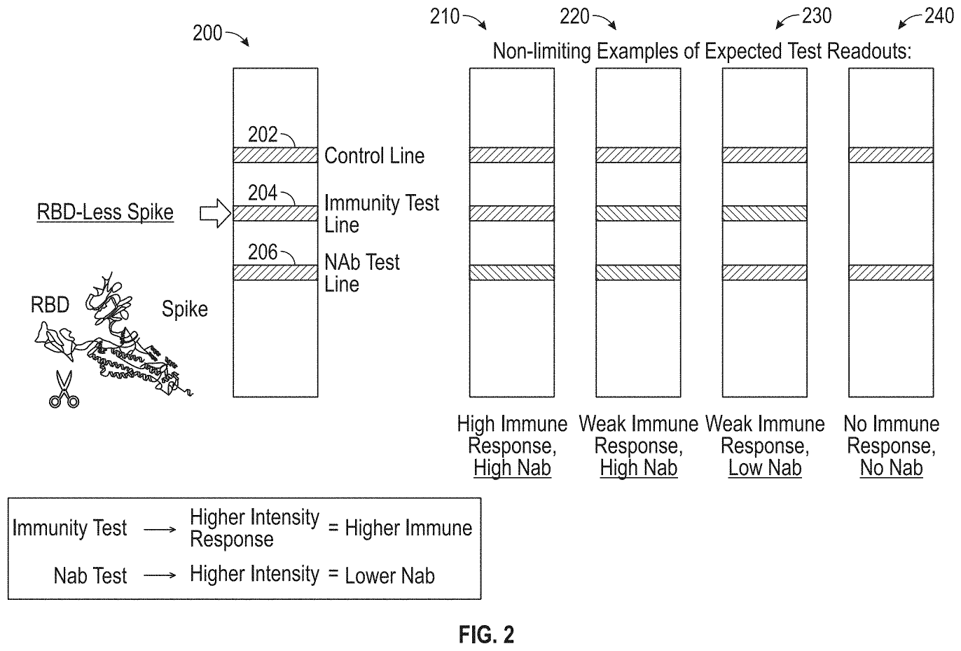

In some embodiments, the functional antibodies are at least one of neutralizing antibodies, blocking antibodies, and enhancing antibodies. In some embodiments, the functional antibodies are neutralizing antibodies, and the binding antibodies are non-neutralizing antibodies. FIG. 2 is a schematic of an exemplary lateral flow immunoassay 200 for detecting neutralizing antibodies and non-neutralizing antibodies, along with non-limiting examples of potential test results, according to an embodiment.

Lateral flow assays are based on the principles of immunochromatography and can be used to detect, quantify, test, measure, and monitor a wide array of analytes, pathogens (e.g., SARS-CoV-2), and the like. Neutralizing antibodies identified using the disclosed methods can specifically bind to any known or as yet undiscovered virus, including for example, coronavirus (e.g., coronavirus OC43, coronavirus 229E, coronavirus NL63, coronavirus HKU1, MERS-CoV, SARS-CoV, or SARS-CoV-2 (COVID-19)). In some embodiments, the neutralizing antibodies are directed against SARS-CoV-2 (COVID-19). In the context of the present disclosure a “neutralizing antibody” is an antibody that binds to a virus (e.g., a coronavirus) and interferes with the virus' ability to infect a host cell. A non-neutralizing antibody is an antibody that binds to a virus but does not interfere with the virus' ability to infect a host cell. Coronavirus Spike proteins are known to elicit potent neutralizing-antibody and T-cell responses. The ability of a virus (e.g., coronavirus OC43, coronavirus 229E, coronavirus NL63, coronavirus HKU1, MERS-CoV, SARS-CoV, or SARS-CoV-2 (COVID-19)) to gain entry into cells and establish infection is mediated by the interactions of its “viral-ACE2 binding protein” (e.g., Spike glycoproteins, and the like) with human cell surface receptors.

As shown on the far left of FIG. 2, the immunoassay 200 can comprise a control line 202, an immunity test line or region 204, and a nAb test line or region 206. In some embodiments, the control line is downstream of the immunity test line, which is downstream of the nAb test line.

The immunity test region can comprise an anti-tag of a first tag/anti-tag pair that is bound to the immunity test region. A corresponding tag of the first pair can be bound to a coupling molecule, which can comprise a non-essential portion of the viral-ACE2 binding protein (e.g., an RBD-less Spike protein, an RBM-less Spike protein). Another molecule, which similarly to the coupling molecule, comprises the non-essential part of a viral-ACE2 binding protein (e.g., another RBD-less Spike protein, another RBM-less Spike protein) can be coupled to a label.

A binding antibody (here, the non-neutralizing antibody) of a test sample, if present, binds to each of the labeled molecule comprising the non-essential part of the given protein (here, Spike protein) and the tagged coupling molecule (which can also comprise the non-essential part of the given protein) to form a complex of the binding antibody (here, non-neutralizing antibody), the labeled molecule comprising the non-essential part, and the tagged coupling molecule (an “A-B-C complex”), and the immunity test line can capture the A-B-C complex via the first tag/anti-tag pair.

The neutralizing antibodies test region can comprise a target (e.g., ACE2 or a fragment thereof). In some embodiments, ACE2 can be bound directly to the nAb test region via covalent bonding. In an embodiment, the covalent bond is amine-glutaraldehyde-amine, where an amine group on ACE2 is conjugated to an amine group either natively present or introduced on the surface of the nitrocellulose membrane. In an embodiment, the covalent bond is amine-NHS (N-hydroxysuccinimide), where NHS ester is used as a covalent linking agent. In an embodiment, the covalent bond is carboxylate-1-ethyl-3-(3-dimethylamonipropyl) carbodiimide (EDC)-amine, where carbodiimide is used to form amide linkage between carboxylates and amines. In other embodiments, the covalent bond is carboxylate-EDC+NHS-amine. In an embodiment, the covalent bond is amine/sulfhydryl-epoxide, where epoxides form covalent bonds with primary amines at mild alkaline pH or with sulfhydryl groups (—SH) in the physiological pH range. In an embodiment, the covalent bond is amine-isothiocyanate, where the reaction of an aromatic amine with thiophosgene (CSCl2) yields isothiocyanate (—NCS), which forms a stable bond with primary amine groups. In another embodiment, the covalent bond is amine-azlactone, where azlactone is used to react with nucleophiles such as amines and thiols at room temperature to form amide bonds. In an embodiment, the covalent bond is amine-p-nitrophenyl ester, where p-nitrophenyl ester is reactive to amines across the slightly basic pH range spanning 7-9 and the ester forms a stable amide bond with proteins. In an embodiment, the covalent bond is amine-tyrosinase (TR)-tyrosine. Tyrosinase is a phenol oxidase that oxidizes phenols into 0-quinone (i.e., 1,2-benzoquinone), which is reactive and undergoes reaction with various nucleophiles such as primary amines. In another embodiment, the covalent bond can be sulfhydryl-maleimide, where maleimide is used to form covalent links with the cysteine residues of proteins. In another embodiment, the covalent bond is reactive hydrogen-benzophenone, where during UV exposure, the benzophenone couples with a protein via reactive hydrogen compounds on the protein. When the benzophenone residues are incorporated onto sample pad, the ACE2 can be immobilized to the surface of the sample pad via exposure to UV light. The particular methods of applying these covalent bonding chemistries to conjugation of proteins is known to those of skill in the art. Multiple covalent bonding chemistries can be used together, including with bifunctional linkers, as known to those of skill in the art. An enormous variety of covalent conjugation chemistries beyond those listed here are known to those of skill in the art. See, for example Kim et al. Biomicrofluidics 7, 041501 (2013), Rusmini et al. Biomacromolecules 8, 1775 (2007), and Hermansson Bioconjugate Techniques, 2nd ed. (Academic Press, San Diego, 2008), all incorporated herein by reference.

The covalent bonding chemistries described above are useful not only for directly conjugating ACE2 to the nitrocellulose membrane, but also for conjugating the respective molecules for noncovalent interactions to ACE2 or to the nitrocellulose membrane, for example for conjugating biotin to ACE2 and/or for conjugating avidin or streptavidin to the nitrocellulose membrane. Additionally, spacers such as polyethylene glycol (PEG) chains can be used together with the linkers for such covalent conjugation (e.g., PEG-NHS) to provide space between the ACE2 and nitrocellulose membrane, and/or ACE2 and biotin, and/or avidin or streptavidin and nitrocellulose membrane. Such spacing can be used to provide the ACE2 with more freedom of movement relative to the nitrocellulose membrane and thus greater opportunity to interact with the viral ACE2-binding protein and/or neutralizing antibodies.

In some embodiments, ACE2 can bind to the nAb test region via a second tag/anti-tag pair. In some embodiments, ACE2 or a fragment thereof is bound to biotin (tag), and streptavidin (anti-tag) is bound to the nAb test region.

As used herein the term “tag/anti-tag pair” or vice versa (anti-tag/tag pair) refers to 2 moieties that are known to bind (e.g., non-covalently) to each other. For example, tag/anti-tag pairs can be ligand/receptor pairs; where the anti-tag is the binding partner to the tag. In an embodiment, the ACE2 or functional fragment thereof (referred to herein as ACE2 for simplicity) binds to the nitrocellulose membrane through a tag/anti-tag interaction during the assay. In another embodiment, the ACE2 is bound to the nitrocellulose membrane through a tag/anti-tag interaction prior to the assay, for example during manufacturing of or preparation of the assay. In an embodiment, the tagged coupling molecule binds to the nitrocellulose membrane through a tag/anti-tag interaction during the assay. In another embodiment, the tagged coupling molecule is bound to the nitrocellulose membrane through a tag/anti-tag interaction prior to the assay, for example during manufacturing of or preparation of the assay.

The tag/anti-tag interaction can be a noncovalent interaction, such as a protein-ligand interaction. In an embodiment, the noncovalent protein-ligand interaction is an interaction between biotin and avidin or streptavidin. Biotin (or other tag) is conjugated to the ACE2 (or other tagged molecule), and avidin or streptavidin (or other anti-tag) is conjugated to the nitrocellulose membrane. For example, with biotin-ACE2 and streptavidin-conjugated nitrocellulose membrane, the high-affinity interaction between biotin and avidin or streptavidin tethers the biotin-ACE2 conjugate to the streptavidin-conjugated nitrocellulose membrane such that the ACE2 is then available to be bound by the viral ACE2-binding protein from the conjugate pad. Streptavidin is a tetramer and each subunit binds biotin with equal affinity; thus, wild-type streptavidin binds four biotin molecules. For some applications it is useful to generate a strong 1:1 complex of two molecules, i.e., monovalent binding. Monovalent streptavidin is an engineered recombinant form of streptavidin which is still a tetramer but only one of the four binding sites is functional. A streptavidin with exactly two biotin binding sites per tetramer (divalent streptavidin) can be produced by mixing subunits with and without a functional biotin binding site. A streptavidin with exactly three biotin binding sites per tetramer (trivalent streptavidin) can also be produced using the same principle as to produce divalent streptavidins. The streptavidin used in the inventive assay can be wild-type (binding four biotins), or it may be monovalent, divalent, or trivalent. Methods of conjugating biotin and streptavidin to proteins and substrates are known to those of skill in the art and any such methods can be used to conjugate biotin or streptavidin to ACE2, and to conjugate biotin or streptavidin to the sample pad.

In another embodiment, the noncovalent protein-ligand interaction is a Halo interaction. Halo-Tag is a 33 kDa mutagenized haloalkane dehalogenase that forms covalent attachments to substituted chloroalkane linker derivatives (Halo-Ligand). Similarly to the streptavidin-biotin connection, the chloroalkane linker extends 1.4 nm into the hydrophobic core of Halo-Tag. Commercially available Halo-ligand derivatives include the invariant chloroalkane moiety followed by 4 ethylene glycol repeats, and a reactive sulfahydryl, succinimidyl ester, amine, or iodoacetamide group, among many other options. Methods of conjugating biotin and streptavidin to proteins and substrates are known to those of skill in the art and any such methods can be used to conjugate Halo-Tag or Halo-Ligand to ACE2 and/or coupling molecule, and to Halo-Tag or Halo-Ligand to the nitrocellulose membrane.

In another embodiment, the noncovalent protein-ligand interaction is a His-tag interaction. The His-tag (also called 6×His-tag) contain six or more consecutive histidine residues. These residues readily coordinate with transition metal ions such as Ni2+ or Co2+ immobilized on beads or a resin. The His-tag is added to the recombinant ACE2 and/or coupling molecule used in the assay, with the beads or resin with immobilized Ni2+ or Co2+ conjugated or otherwise bound to the nitrocellulose membrane. Methods of adding His-tags to proteins and beads or resin with immobilized Ni2+ or Co2+ to substrates are known to those of skill in the art and any such methods can be used to add a His-tag to ACE2 and/or coupling molecule, and beads or resin with immobilized Ni2+ or Co2+ to the nitrocellulose membrane. In other embodiments, the noncovalent interaction utilizes a ligand tag that is calmodulin-binding peptide, glutathione, amylose, a c-my tag, or a Flag tag. The ligand tag is attached to the ACE2 and/or coupling molecule, and the respective ligand-binding molecule is attached to the nitrocellulose membrane using methods known to those of skill in the art. The ligand tag can also be single-strand DNA (ssDNA) attached to the ACE2 and/or coupling molecule, where the complementary ssDNA is immobilized on the nitrocellulose membrane.

In some embodiments, the conjugate pad can comprise a mixture of (a) a first molecule coupled to a first label, the first molecule comprising essential part of the viral-ACE2 binding protein (e.g., RBD or RBM region of a SARS-CoV-2 Spike protein), (b) a coupling molecule bound to a tag, the coupling molecule comprising non-essential part of the viral-ACE2 binding protein (e.g., RBD-less or RBM-less Spike protein), (c) a second molecule coupled to a second label, the second molecule comprising the non-essential part of a viral-ACE2 binding protein (similarly to the coupling molecule), and/or (d) a target bound to a second tag (e.g., ACE2-biotyn).

In some embodiments, the label(s) is/are selected from a nanoparticle, bead, latex bead, aptamer, and/or a quantum dot. As used herein, the term “label” refers to a moiety, the presence of which can be detected using methods well-known in the art for label-detection. In an embodiment, the first molecule comprising essential part of the viral ACE2-binding protein is coupled to a label such that it can be detected when bound to the ACE2 bound to the nitrocellulose membrane, thus demonstrating a lack of neutralizing antibodies in the sample. In an embodiment, the control protein (for example, an anti-IgG monoclonal antibody) is coupled to a label such that it can be detected when bound to its target on the nitrocellulose membrane (for example, IgG on the Control Line), thus demonstrating that the test is functional and has been performed properly. In an embodiment, the first molecule comprising the essential part of the viral ACE2-binding protein, the second molecule comprising the non-essential part of the viral-ACE2 binding protein, and the control protein are coupled to different labels. In an embodiment, the label for the first molecule, the second molecule and/or that for the control protein is detectable by the naked eye. In another embodiment, the label for the first molecule, the second molecule and/or that for the control protein is detectable by fluorescence. In another embodiment, the label for the first molecule, the second molecule and/or that for the control protein is detectable by chemiluminescence. Methods for coupling the labels to proteins are known to those of skill in the art.

Labels detectable by the naked eye include metal nanoparticles and nanoshells (e.g., green gold nanoshells; red, orange, or blue gold nanoparticles; copper oxide nanoparticles; silver nanoparticles), gold colloid, platinum colloid, polystyrene latex or natural rubber latex colored with respective pigments such as red and blue. Labels detectable by the naked eye include textile dyes, such as for example, a Direct dye, a Disperse dye, a Dischargeable acid dye, a Kenanthol dye, a Kenamide dye, a Dyacid dye, a Kemtex reactive dye, a Kemtex acid dye, a Kemtex Easidye acid dye, a Remazol dye, a Kemazol dye, a Caledon dye, a Cassulfon dye, an Isolan dye, a Sirius dye, an Imperon dye, a phtalogen dye, a naphtol dye, a Levafix dye, a Procion dye, and an isothiocyanate dye. Examples of textile dyes that can be used to label proteins include, for example, Remazol brilliant blue, Uniblue A, malachite green isothiocyanate, and Orange 16 (Remazol orange). Any label known to those of skill in the art to both be fluorescent and used to label proteins can be used.

Fluorescent labels include any of the Alexa fluor dyes, any of the BODIPY dyes, any of the eFluor dyes, any of the Super Bright dyes, fluorescein or a derivative thereof, eosin or a derivative thereof, tetramethylrhodamine, rhodamine or a derivative thereof, Texas red or a derivative thereof, pyridyloxazole or a derivative thereof, NBD chloride, NBD fluoride, ABD-F, lucifer yellow or a derivative thereof, 8-anilino-1-naphthalenesulfonic acid (8-ANS) or a derivative thereof, Oregon green or a derivative thereof, Pacific blue or a derivative thereof, Pacific green or a derivative thereof, Pacific orange or a derivative thereof Cy3, Cy5, Cyanine7, Cyanine5.5, or coumarin or a derivative thereof. Fluorescent labels include any fluorescent protein, such as green fluorescent protein (GFP), red fluorescent protein (e.g., dsRed), cyan fluorescent protein, blue fluorescent protein, yellow fluorescent protein, enhanced green fluorescent protein (EGFP), or any derivative of such fluorescent proteins thereof. Any label known to those of skill to both be fluorescent and be used to label proteins can be used.

Chemiluminescent labels include enzyme labels that catalyze formation of ATP which is then assayed by the firefly reaction or that catalyze formation of peroxide which is determined by luminol, isoluminol, or peroxyoxalate CL. Such enzyme labels include luciferase and horseradish peroxidase. Any label known to those of skill in the art to both be chemiluminescent and used to label proteins can be used.

In some embodiments, the first molecule is coupled to a gold nanoshell (GNS) and the second molecule is coupled to a gold nanosphere (GNP). In some embodiments, reading the results from the test-cassette/assay further comprises determining the intensity of a test region (e.g., test line) in the assay compared with a reference standard. In a particular embodiment, the reference standard is a scorecard. As used herein, the phrase “reference standard” refers to a control set of values, either obtained simultaneously with the assay results or obtained from a previous control experiment such that they are indicative of the level of functional antibodies (e.g., nAb) and/or binding antibodies (e.g., non-nAbs) present in the test-specimen. In a particular embodiment, the reference standard is a scorecard.

In the examples shown, a higher intensity on the immunity test line corresponds to a higher immune response/higher levels of non-nAbs associated with the test sample, and a higher intensity on the nAb test line corresponds to lower levels of nAbs in the test sample.

In certain embodiments, the level of functional antibodies such as nAbs (e.g., anti-SARS-CoV-2 nAbs) in the test-specimen is reported as falling within a range of pre-determined values. In certain embodiments, the level of binding antibodies (e.g., non-nAbs) in the test-specimen is reported as falling within a range of pre-determined values. In some embodiments, the range of pre-determined values corresponds to High (H), Moderate-High (MH), Moderate to Moderate-High (M-MH), Moderate (M), Moderate to Not Detectable (M-ND) and Not Detectable (ND). As used herein, the phrase “reported as falling within a range of pre-determined values” refers to the manner in which the level of functional and/or binding antibodies are analyzed relative to the reference standard or set of control values. The range of pre-determined values can be as few as two levels of functional and/or binding antibody values (or concentrations) up top about 10 or more distinct concentration (or quantity) levels of functional and/or binding antibodies present in the test-specimen. In one embodiment corresponding to 2 levels of nAb values, for example, falling either above or below a predetermined set value may indicate the presence of sufficient protective anti-RBD nAbs, such that there is a greater likelihood there is protection from getting a subsequent coronavirus infection. Those of skill in the art will appreciated that any number of functional and/or binding antibody concentrations and/or quantity levels can be used to identify particular test-specimens being assayed for particular purposes, e.g., those test-specimens above a specified level can be advantageously useful in convalescent therapy.

In some aspects, measuring nAb (or other fAb) levels, measuring non-nAb (or other bAb) levels, determining a ratio of nAbs (or other fAbs) to non-nAbs (or other bAbs), determining a ratio of nAbs (or other fAbs) to total antibodies, determining a ratio of non-nAbs (or other bAbs) to nAbs (or other fAbs), and/or determining a ratio of non-nAbs (or other bAbs) to total antibodies, can comprise using an electronic device.

### EXAMPLE

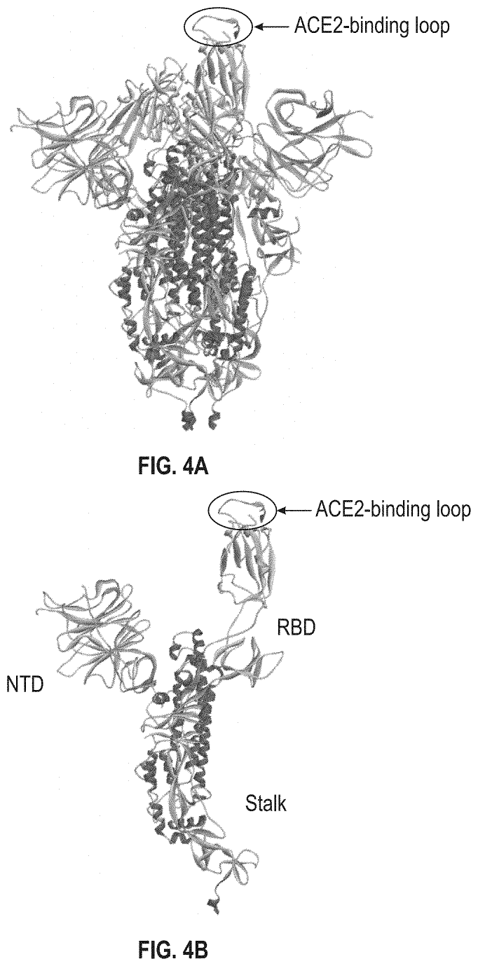

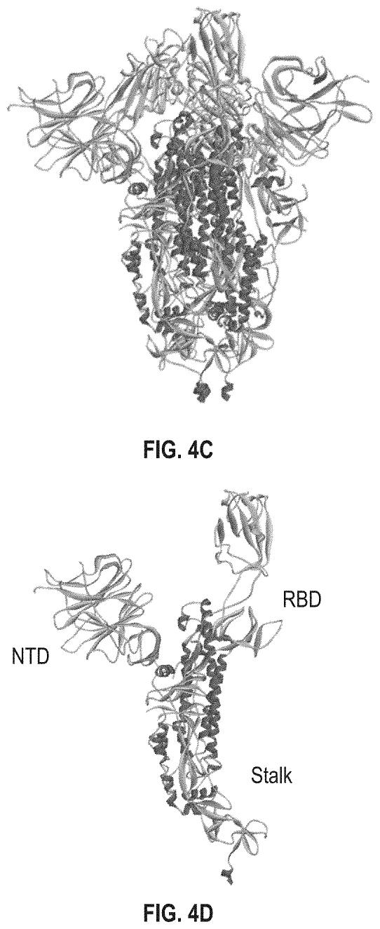

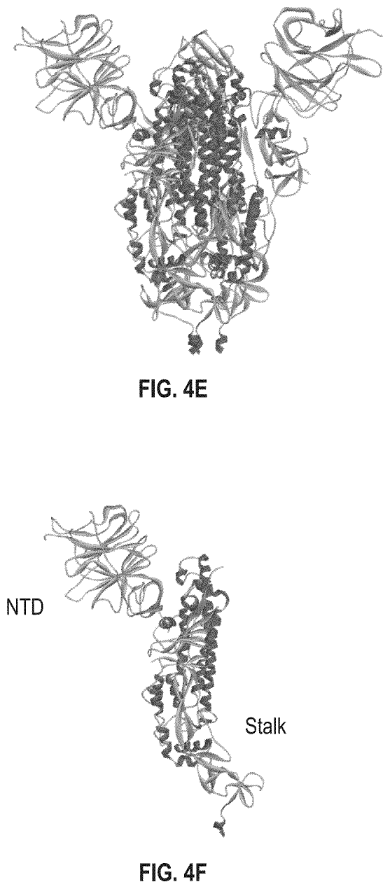

Spatial protein and primary DNA structures were analyzed using Biovia Discovery Studio 2020 Client (Dassault Systems) and Gene Runner (Frank Buquicchio & Michael Spruy) software packages. The human codon-optimized cDNA encoding 1,213 amino-acids of extra-virion domain of SARS-CoV-2 Spike protein with Asp614Gly, Arg682Gly, Arg683Gly, Arg685Ser, Lys814Ala, Arg815Gly, Phe817Pro, Ala892Pro, Ala899Pro, Ala942Pro, Lys986Pro, Val987Pro protein stabilizing mutations, followed by a C-terminal T4-phage fibritin trimerization domain and 6×His tag was cloned into BamHI and XhoI sites of pcDNA3.1 expression vector under the control of human CytomegaloVirus (CMV) promoter. Thus, obtained expression vector was then used for the generation of protein variants without T4-phage fibrtin trimerization domain, with the deletion of ACE2-binding loop, amino-acids 447-500 (“Loop-less”) in RBD domain, and for the variant missing the entire RBD, amino-acids 325-591 (“RBD-less”) by PCR assisted techniques. FIG. 4A is a schematic of a Spike protein trimer with an ACE2 receptor binding domain (or the “RBD”) including ACE2 binding loop (or “full length Spiker protein trimer”) according to an embodiment. FIG. 4B is a schematic of a Spike protein monomer with an ACE2 receptor binding domain including ACE2 binding loop (or “full length Spiker protein monomer”) according to an embodiment. FIG. 4C is a schematic of a Spike protein trimer with an ACE2 receptor binding domain not including an ACE2 binding loop (or “loop-less Spike protein trimer”) according to an embodiment. FIG. 4D is a schematic of a Spike protein monomer with an ACE2 receptor binding domain not including ACE2 binding loop (or “loop-less Spike protein monomer”) according to an embodiment. FIG. 4E is a schematic of a Spike protein trimer without an ACE2 receptor binding domain (or “RBD-less Spike protein trimer”) according to an embodiment. FIG. 4F is a schematic of a Spike protein monomer without an ACE2 receptor binding domain (or “RBD-less Spike protein monomer”) according to an embodiment. The integrity of obtained expression vectors was verified by automated DNA sequencing.

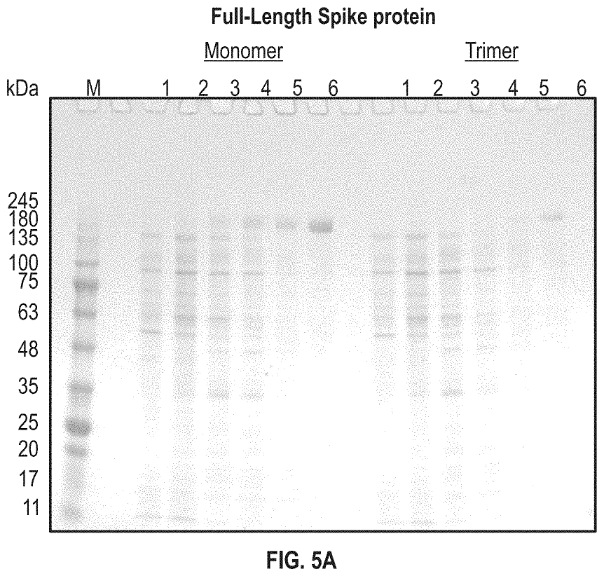

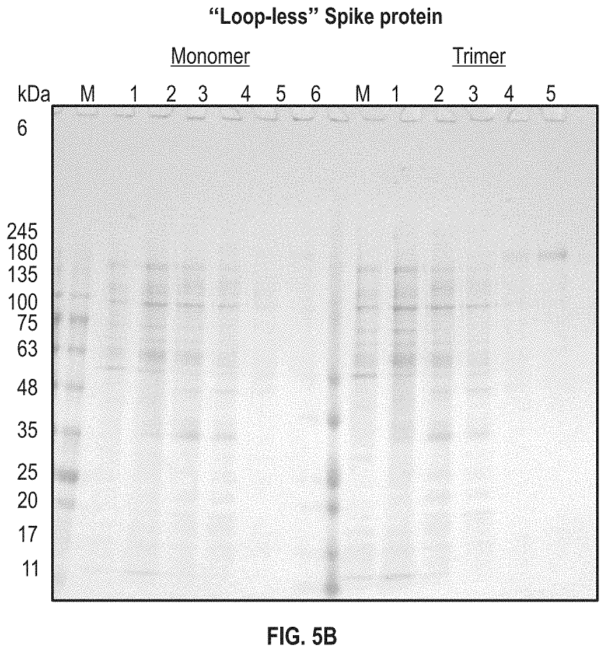

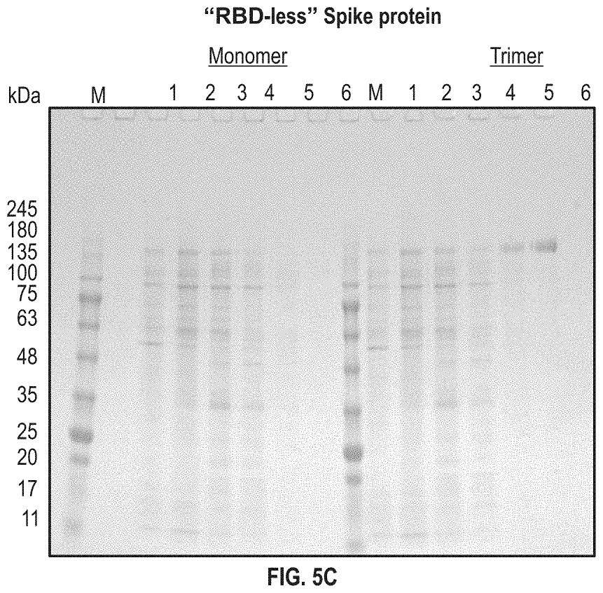

Protein expression and purification feasibility were evaluated at a small scale by PEI-mediated transient transfection of endotoxin-free vectors encoding Spike protein variants into 50 mL Human embryonic kidney 293-F cells (HEK293F cell) cultures at 0.7×10⁶ cells/L −0.8×10⁶ cells/L, grown in 250 mL vented shaker flasks at 130 RPM, 37° C. in a 5% CO₂ humidified atmosphere. Transfected cells were cultured for six days before protein purification from cell culture medium samples. Proteins were purified by immobilized metal affinity chromatography (IMAC) using 0.25 mL of His60 Ni Superflow resin (TaKaRa) by gravity flow method following resin manufacturer recommendations. Resin-bound protein samples were extensively washed and eluted by a step gradient of imidazole from 10 mM to 320 mM. The purity of eluted fraction was analyzed by reducing 4-20% SDS-PAGE stained with GelCode Blue (ThermoFisher Scientific). FIG. 5A is 4-20% reducing SDS-PAGE analysis of a full length Spike protein variants expressed in HEK293F cells at 50 mL scale and purified by IMAC. FIG. 5B is 4-20% reducing SDS-PAGE analysis of a loop-less Spike protein variants expressed in HEK293F cells at 50 mL scale and purified by IMAC. FIG. 5C is 4-20% reducing SDS-PAGE analysis of a RBD-less protein variants expressed in HEK293F cells at 50 mL scale and purified by IMAC. In FIGS. 5A-5C, “M” indicates a molecular weight (MW) protein marker, “1” indicates an eluted fraction with 10 mM Imidazole fraction, “2” indicates an eluted fraction with 20 mM Imidazole fraction, “3” an eluted fraction with indicates 40 mM Imidazole fraction, “4” an eluted fraction with indicates 80 mM Imidazole fraction, “5” indicates an eluted fraction with 160 mM Imidazole fraction, and “6” indicates an eluted fraction with 320 mM Imidazole fraction. The main protein peak was eluted with 320 mM Imidazole in all cases except monomers of RBD-less and Loop-less variants that yielded no or very low amount of target protein (a protein band migrating between 135 and 180 kDa). Referring to FIGS. 5A-5C, out of six variants tested at a small scale, only full-length Spike monomer, full-length Spike trimer, RBD-less, Spike trimer, and Loop-less Spike trimer proteins gave satisfactory yields judging by the SDS-PAGE analysis of the IMAC purified protein fractions. Referring to FIGS. 5A-5C, the purified protein fractions were analyzed in 4-20% gradient denaturing poly-acrylamide gel (4%-20% SDS-PAGE) under the reducing conditions. Under such conditions proteins will migrate as single bands regardless their quaternary structure.

Full-length Spike monomer, RBD-less Spike trimer, and Loop-less Spike trimer variants were chosen for protein expression and purification at a 1 L scale that was performed as described above with the following modifications. Before transfection, 1 L cell cultures were grown in 3 L flasks. Proteins were purified on 3 mL columns connected to the FPLC system, and during the elution step, the imidazole gradient was extended to 640 mM and 1000 mM. The major protein peak fractions eluted from columns with 160 mM 640 mM imidazole were pooled, buffer exchanged into PBS, pH 7.2, and concentrated to 1 mg/mL measured by absorption at 280 nm. Then protein samples were aliquoted, stored at −80° C. and used for assay development. The total protein yields varied between 1 and 3 mg/L.

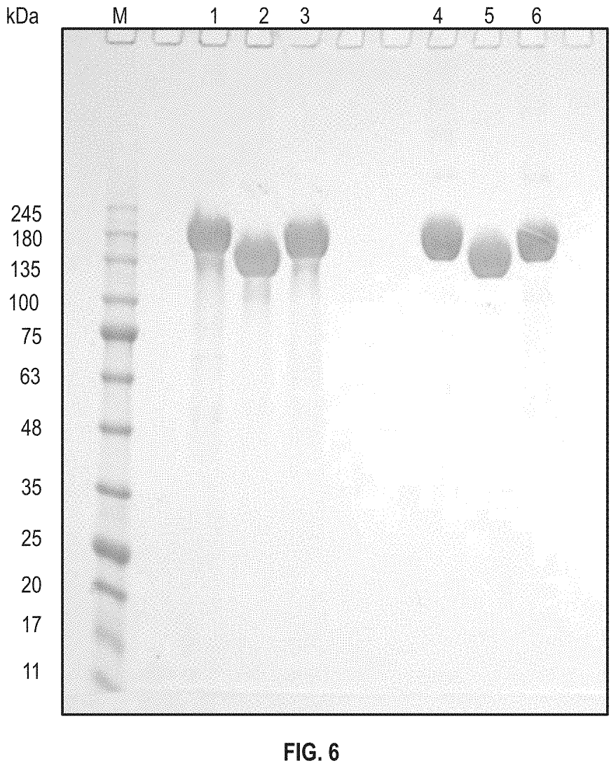

The purity and integrity of protein samples purified at 1 L scale were analyzed in reducing and non-reducing 4-20% SDS-PAGE stained with GelCode Blue and was estimated to be close to or above 90%. FIG. 6 is reducing and non-reducing 4-20% SDS-PAGE analysis of Spike protein variants expressed in HEK293F cells at 1 L scale and purified by IMAC. The protein load was 3 μg per lane on SDS-PAGE. In FIG. 6, “MW” indicates a protein molecular weight marker, “1” indicates full-length Spike variant(reduced), “2” indicates “RBD-less” Spike variant (reduced), “3” indicates “Loop-less” Spike (reduced), “4” indicates Full-length Spike variant (non-reduced), “5” indicates “RBD-less” Spike (non-reduced), “6” indicates “Loop-less” Spike variant (non-reduced). Referring to FIG. 6, it revealed the absence of any major contaminating protein bands of lower and higher molecular weight or protein aggregates. All samples migrated in SDS-PAGE under both reducing and non-reducing conditions as a single protein bands with the apparent molecular weights between 135 and 180 kDa that were slightly higher than the calculated weights of monomeric variants, most likely due to posttranslational glycosylation reported in the literature. In the absence of the reducing agent (e.g., without DTT non-reduction conditions), and in the presence of SDS, Spike proteins did not form trimers that could be visualized as single protein bands with the apparent molecular weight above 400 kDa in SDS-PAGE.

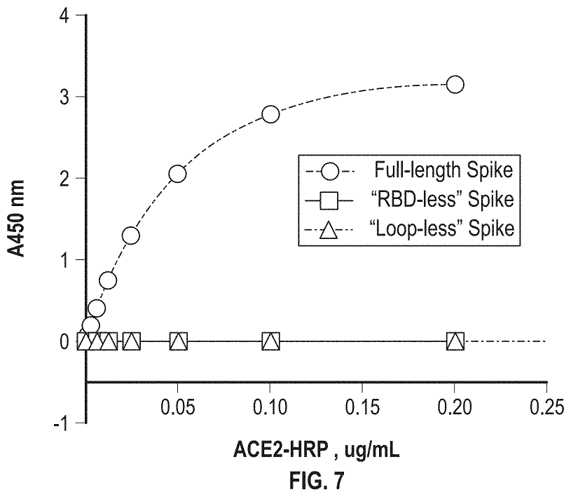

The ACE2 binding activity of purified full-length, “RBD-less” and “Loop-less” Spike protein variants was assessed by direct ELISA with ACE2-HRP conjugate. In brief, proteins were coated in a high-binding 96-well ELISA plate in PBS pH 7.2 at 1 ug/mL, 100 uL/wells overnight at 4° C., in duplicates. The next day plate was washed with ELISA wash buffer (PBS-Tween 20, 0.05%), and blocked for 1 hour at room temperature with ELISA blocking buffer (10 mg/mL BSA in PBS-Tween 20, 0.025%). After that plate was washed with 300 uL/well of ELISA wash buffer and incubated with 100 uL/well of two-fold serial dilutions of ACE2-HRP conjugate in ELISA blocking buffer, starting from 0.2 ug/mL for 1 hour at room temperature. The plate was washed again, and bound ACE2-HRP was detected in the presence of TMB HRP-substrate for 5 min. Then HRP reaction was stopped with 50 uL/well of 2M sulfuric acid, and absorbance in wells was read at 450 nm. The 450 nm absorption values with average blanks subtracted were analyzed and plotted in GraphPad Prizm software (Dotmatics) using a nonlinear regression binding-saturation algorithm. FIG. 7 is a graph indicating evaluation of ACE2-binding activity of full-length, “RBD-less” and “Loop-less” Spike proteins by a direct ELISA. Referring to FIG. 7, both “RBD-less” and Loop-less” Spike variant proteins showed complete loss of ACE2-binding activity.

In some embodiments, different size variants of the protein of interest may provide different features for different applications. For example, a relatively larger size variant may allow more binding sites for a bAb, which, for example, may make the bAb-based signal stronger. For example, a relatively smaller size variant may allow a smaller size-based or a lighter weight molecule-based application. For example, a Spike protein trimer variant may allow more binding sites for a bAb to bind compared to a Spike protein. For example, a loop-less Spike protein variant may provide more binding sites for a bAb to bind compared to a RBD-less Spike protein.

In some embodiments, a method can comprise scanning a code into the electronic device that identifies a test to be performed and a particular specimen to be tested, performing the method of detecting the presence and/or quantity of nAbs (fAbs) and non-nAbs (bAbs) according to methods provided herein for detection of antibodies in a test-specimen, and scanning the results obtained from the test-cassette into the electronic device. In some embodiments, the results are processed directly on the electronic device. In other embodiments, the electronic device further connects to a database, thereby transferring the results to said database. In certain embodiments, the device connects to the database via WiFi, SMS, worldwide web, 4G, 5G, Bluetooth and/or USB.

The electronic device can be at least one of a desktop personal computer, laptop or notebook personal computer, tablet computer, personal digital assistant, smartphone, smartwatch, smartcard, bracelet, smart clothing item, smart jewelry, media internet device, head-mounted display, or wearable glasses.

In other embodiments, the electronic device may include an operating system (OS) serving as an interface between hardware and/or physical resources of the electronic device and a user. The electronic device may include one or more processors, memory devices, network devices, drivers, or the like, as well as input/output (I/O) sources, such as touchscreens, touch panels, touch pads, virtual or regular keyboards, virtual or regular mice, and the like.

In particular embodiments, the electronic device into which the test results are scanned may be in communication with another electronic device, serving as a central computer or server computer, over one or more networks, such as a Cloud network, the Internet, intranet, Internet of Things (“IoT”), proximity network, wireless/cellular communication network (such as 3G, 4G, 5G, and/or 6G), Bluetooth, etc. Further, the electronic device into which the test results are scanned and/or the central or server computer may be in communication with one or more third-party electronic devices over the one or more networks. The central computer or server computer can be used to store, organize, keep track of, and/or analyze the test results scanned into multiple electronic devices. The third-party electronic devices can be used to access the data regarding the test results from the central computer or server computer, and/or to further analyze or utilize such data.

In other embodiments of the inventive method, the electronic device may transfer the test results to a database. The database may be contained in a central computer or server computer, or distributed across multiple electronic devices. To transfer test results, the electronic device may connect to the database via WiFi, WiMax, SMS, the Internet (including worldwide web), intranet, Internet of Things (“IoT”), proximity network, wireless/cellular communication network (such as 3G, 4G, 5G, and/or 6G), Cloud network, Bluetooth and/or USB (such as USB-A, USB-B, and/or USB-C). Results can also be downloaded from the electronic device for transfer to the database via storage media such as a USB flash drive, flash memory card, or SD memory card. The database may store and maintain any amount and type of data including but not limited to the presence or absence of functional antibodies, the presence or absence of binding antibodies, relative level of functional antibodies and/or binding antibodies, presence or absence of control or test line colors (including that expressed as density units), color intensity for the control line (including that expressed as density units), color intensity (or other characteristic) for one or more test lines, ratio values associated with functional and binding antibodies and associated IC50 values, interpretations of the test results, estimated antibody titers, sample metadata, and/or other sample data such as patient demographic or genomic data, or patient vaccination and/or infection data.

In the first example test readout (far left example of expected test readouts, 210) of FIG. 2, the test sample comprises a high titer of non-nAbs, which bridge the tagged non-essential part of the Spike protein and the labeled coupling molecule, and wherein the tag of the A-B-C complex binds to the anti-tag of the immunity region. The test sample also comprises a high titer of nAbs, which block the binding of RBD (e.g., labeled RBD of the test kit) to ACE2 (or fragment thereof). In the second example test readout 220, the test sample comprises low titer of non-nAbs and a high titer of nAbs. In the third example test readout 230, the test sample comprises a low titer of non-nAbs and a low titer of nAbs. In the fourth example (far right) test readout 240, the test sample comprises no non-nAbs and no nAbs, indicating the test sample is from an individual that has not had COVID-19.

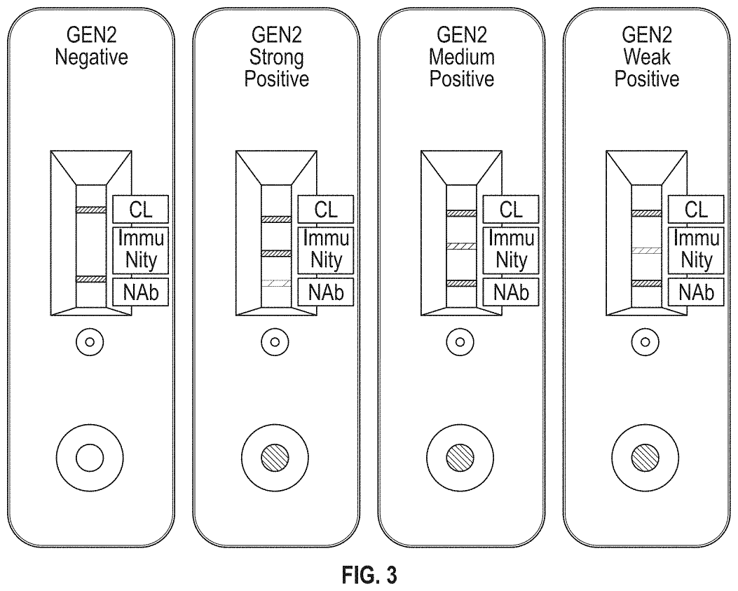

FIG. 3 shows images of actual test results obtained using test kits as described above. The assays each comprise a nAb test line, an immunity test line, and a control line, each of which are spatially separated from each other. The tests were performed with fingerstick blood using donors with varying immunity to SARS-CoV-2. The assay of FIGS. 2 and 3 can be used to measure levels of neutralizing antibodies against Spike protein receptor binding domains (RBD) that block the RBDs from binding to ACE2 receptors, and levels of non-neutralizing antibodies against Spike proteins. The addition of serum or plasma lacking nAbs (far left of FIG. 3) does not block binding of RBD-beads to ACE2 resulting in the RBD-bead—ACE2 complex creating a visible line at the test location (e.g., nAb test line). The addition of serum or plasma lacking non-nAbs (far left of FIG. 3) does not form a A-B-C complex, resulting in no visible line at the immunity test line. This “negative” result shows no color on the immunity test region and a high intensity color on the nAb test region from a test sample comprising no non-nAbs and no nAbs. The “strong positive” results show a high intensity color on the immunity test region and low intensity color on the nAb test region from a test sample comprising a high titer of non-nAbs and nAbs. The “medium positive” results show medium (between weak and strong) intensity color on the immunity test region and a medium to strong intensity color on the nAb test region from a test sample comprising a medium titer of non-nAbs and low to medium titer of nAbs. The “weak positive” results show low intensity color on the immunity line and medium to high intensity color on the nAbs test region from a test sample comprising a low titer of non-nAbs and low titer of nAbs.

The control location (e.g., control line) downstream of the test lines represents capture of gold nanospheres (or other label) coupled to a monoclonal antibody (e.g., a mouse Mab, or the like).

Detection and measuring of non-neutralizing antibodies can provide information on the presence of general innate immune response. Detection and measuring of nAbs can determine serum neutralizing activity. The ratio of nAbs/non-nAbs can provide the percent of protective antibodies. In some embodiments, the assays described herein that can measure the ratio of total and neutralizing antibodies and/or non-neutralizing and neutralizing antibodies for other pathogens. In some embodiments, the assays described herein can measure the ratio of total and functional antibodies and/or binding and functional antibodies for endogenous or therapeutic bioactive proteins. In some embodiments, the assays described herein can provide pattern recognition of weak, medium and strong neutralizers.

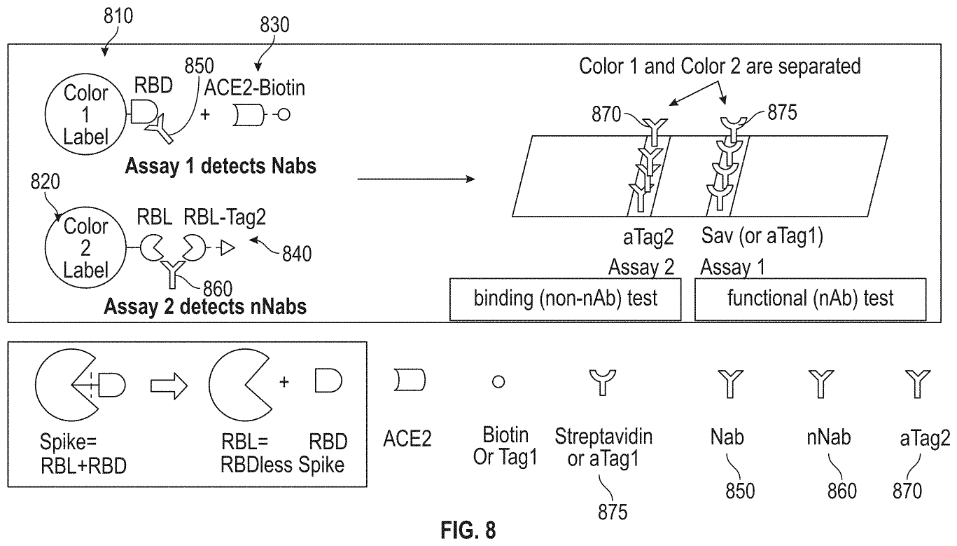

FIG. 8 is a schematic of a bi-color immunoassay and molecules of a test kit, according to an embodiment. The test kit of FIG. 8 comprises an assay, here a lateral flow assay, comprising a sample receiving portion (e.g., a sample receiving port, a sample pad, and/or a sample filter), and a detection zone. In some embodiments, the test kit comprises at least one of a sample receiving portion and a conjugate release pad that holds one or more of a labeled first molecule 810 comprising an essential part of a protein (here, RBD domain of a Spike protein), a labeled second molecule 820 comprising a non-essential part of the protein (here, RBD-less and/or RBM-less Spike protein), a tagged target molecule 830 (here, ACE2), and a tagged coupling molecule 840 (here, another RBD-less and/or RBM-less Spike protein). In particular embodiments, the target is spatially separated from the first molecule, the second molecule, and the coupling molecule. In one embodiment, the following reagent configuration is employed. A sample pad or sample filter is infused with the tagged target, while conjugate pad is infused with a mixture of the labeled first molecule, the labeled second molecule, the tagged coupling molecule, and a mouse monoclonal antibody coupled to control label as a constant assay control. In another embodiment, the following reagent configuration is employed. A sample pad or sample filter is infused with the tagged target and the tagged coupling molecule, while conjugate pad is infused with a mixture of the labeled first molecule, the labeled second molecule, and a mouse monoclonal antibody coupled to control label as a constant assay control. The purpose of the control bead is to provide reassurances regarding sample addition, reconstitution, and flow. If control line cannot be visualized with the human eye, the test is considered invalid.

When assay (chase) buffer is added to the sample well, the dried components on the strip interact with plasma or serum from whole blood. As a test sample flows through the conjugate release pad, the at least one of the labeled first molecule, the labeled second molecule, the tagged target molecule (here, ACE2), and the tagged coupling molecule are released into the sample. The labeled first molecule binds with the functional (here, neutralizing) antibodies 850 in the test sample, if such functional antibodies are present, and the labeled second molecule and the tagged coupling molecule bind to the binding (here, non-neutralizing) antibodies 860 in the test sample, if present. Labeled first molecules that are not blocked by the functional antibodies will bind to the tagged target (here, ACE2-biotin) and be detectable using methods well-known in the art for label-detection (e.g., creating a high intensity red line). The tagged target (ACE2-biotin) will bind to the anti-tag 875 (here, Streptavidin) on the functional antibodies test region of the detection zone. If the sample contains functional antibodies that prevent the labeled first molecule from binding to the target, the test will show a light or ghost functional test line. If the sample does not contain, or contains low levels of neutralizing antibodies, RBD-gold Nanoshells and ACE2-biotin will interact forming a dark green Test line. If binding antibodies are present in the test sample, they will bridge the tagged coupling molecules and the labeled second molecules, forming an A-B-C complex (a complex comprising 860 bound to 820 and 840). The tagged coupling molecule will bind to anti-tag 2 870 on the binding antibodies test region, and if the binding antibodies bridge the tagged coupling molecules and the labeled second molecules, the presence of such binding antibodies can be detectable using methods well-known in the art for label-detection. If binding antibodies are not present in the test sample, the labeled second molecules will not be captured on the binding antibodies test region.

To perform the test according to one embodiment, 6.8 microliters (ul) (or any other suitable amount) of plasma or serum or 10 ul (or any other suitable amount) of whole blood are applied to the sample pad in the sample port and immediately followed by three drops (˜50 ul) (or any other suitable number of drops or amount) of chase buffer. The plasma/serum+chase buffer reconstitutes target reagent dried in sample pad that then mixes with sample and flows towards the components (e.g., labeled first molecule, labeled second molecule, labeled control molecule, and/or tagged coupling molecule) dried on conjugate pad. Upon flowing through the labeled first molecule, the functional antibody (fAb), if present, competes with the target for binding to the first molecule. The more fAb is present in a sample, the less target-tag can bind to the first molecule. The reaction mixture is drawn by capillary action towards zones striped onto nitrocellulose membrane, separated by ˜5 mm (or any other suitable distance). First is the polystreptavidin (functional antibodies test) zone that rapidly captures any first molecule-label-target-tag complex. Second is second anti-tag comprising binding antibodies test zone that rapidly captures any A-B-C complex. Third, is an optional control zone. In this assay the stronger the signal on the functional test line, the less functional antibodies is present in a sample. Hence, the assay provides a reverse relation between functional antibodies test zone intensity and the amount of functional antibodies in a sample. The stronger the signal on the binding test line, the more binding antibodies is present in the sample.

In some embodiments, reading the results from the test-cassette/assay comprises determining the intensity of a test region (e.g., test line) in the assay compared with a reference standard. In a particular embodiment, the reference standard is a scorecard. The reference standard can refer to a control set of values, either obtained simultaneously with the assay results or obtained from a previous control experiment such that they are indicative of the level of functional antibodies (e.g., nAb) and/or binding antibodies (e.g., non-nAbs) present in the test-specimen.

In some embodiments, a higher intensity on the binding antibodies test line corresponds to a higher level of binding antibodies associated with the test sample, and a higher intensity on the functional test line corresponds to lower levels of functional antibodies in the test sample.

In certain embodiments, the level of functional antibodies such as nAbs (e.g., anti-SARS-CoV-2 nAbs) in the test-specimen is reported as falling within a range of pre-determined values. In certain embodiments, the level of binding antibodies (e.g., non-nAbs) in the test-specimen is reported as falling within a range of pre-determined values.

The assay of FIG. 8 can be used to measure levels of functional antibodies and binding antibodies, including ratios thereof. The addition of serum or plasma lacking functional antibodies does not block binding of the first molecule-beads to the tagged target resulting in the first molecule-bead—target complex creating a visible line at the functional (nAb) test location. The addition of moderate titer fAbs to the sample pad creates a weak line at the fAb test location. The addition of high titer fAbs (>1:640) blocks binding of the first molecule-beads to the target such that no line is observed at the test location on the strip. The control location (e.g., control line) downstream of the test line represents capture of gold nanospheres coupled to a monoclonal antibody (e.g., a mouse Mab, or the like).

Although not illustrated in FIG. 8, some contemplated bi-color immunoassays can further comprise a control region in the detection zone. A control protein (for example, an anti-IgG monoclonal antibody) coupled to a label can be provided (e.g., on the conjugate release pad) such that it can be detected when bound to its target on the nitrocellulose membrane (for example, IgG on the control region), thus demonstrating that the test is functional and has been performed properly.

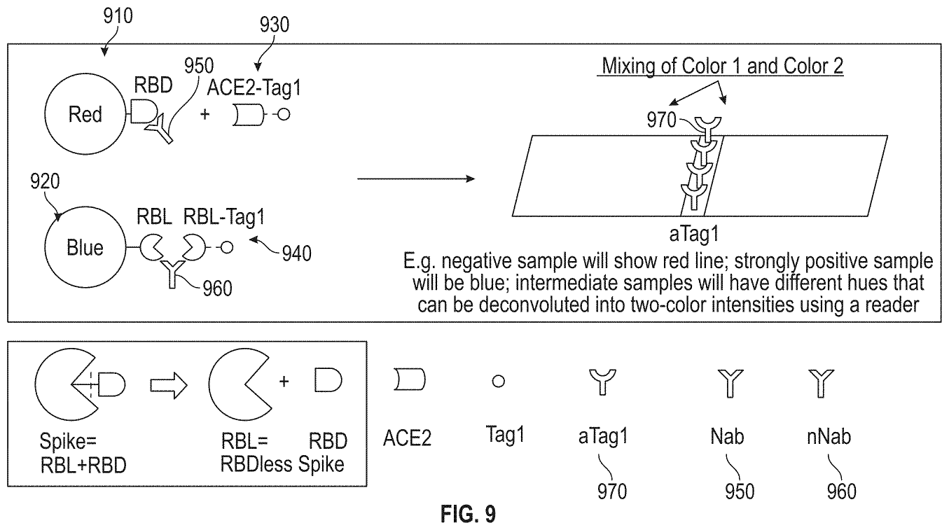

FIG. 9 is a schematic of a mixed color immunoassay and molecules of a test kit (also referred to as CoviHue™), according to an embodiment. The test kit of FIG. 9 comprises an assay, here a lateral flow assay, comprising a sample receiving portion (e.g., a sample receiving port, a sample pad, and/or a sample filter), and a detection zone. In some embodiments, the test kit comprises a sample receiving portion and/or conjugate release pad that holds one or more of a labeled first molecule 910 comprising an essential part of a protein (here, RBD domain of a Spike protein), a labeled second molecule 920 comprising a non-essential part of the protein (here, RBD-less and/or RBM-less Spike protein), a tagged target molecule 930 (here, ACE2), and a tagged coupling molecule 940 (here, another RBD-less and/or RBM-less Spike protein). In particular embodiments, the target is spatially separated from the first molecule, the second molecule, and the coupling molecule. In one embodiment, the following reagent configuration is employed. A sample pad or sample filter is infused with the tagged target, while conjugate pad is infused with a mixture of the labeled first molecule, the labeled second molecule, the tagged coupling molecule, and a mouse monoclonal antibody coupled to control label as a constant assay control. In another embodiment, the following reagent configuration is employed. A sample pad or sample filter is infused with the tagged target and the tagged coupling molecule, while conjugate pad is infused with a mixture of the labeled first molecule, the labeled second molecule, and a mouse monoclonal antibody coupled to control label as a constant assay control. The purpose of the control bead is to provide reassurances regarding sample addition, reconstitution, and flow. If control line cannot be visualized with the human eye, the test is considered invalid.

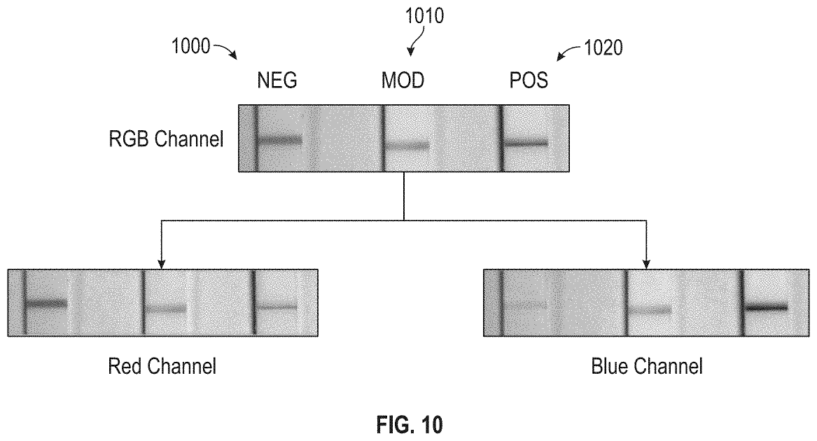

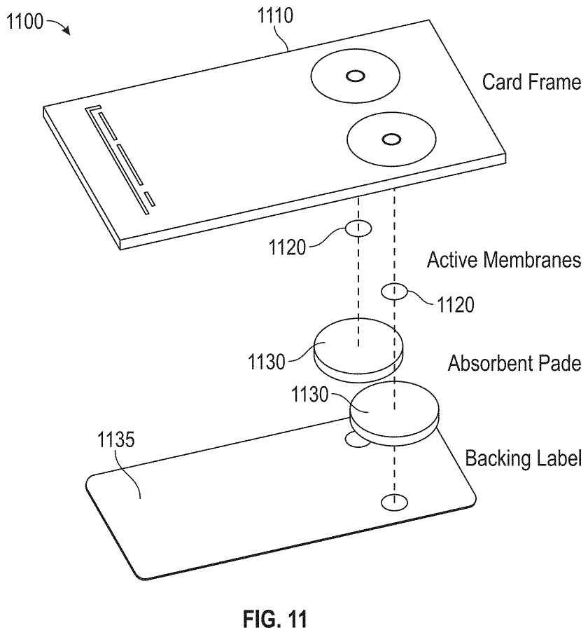

Here, the target molecule is tagged with a first tag, and the coupling molecule is tagged with the same first tag. As a test sample flows through the conjugate release pad, the at least one of the labeled first molecule, the labeled second molecule, the tagged target molecule (here, ACE2), and the tagged coupling molecule are released into the sample. The labeled first molecule binds with the functional (here, neutralizing) antibodies in the test sample, if such functional antibodies are present, and the labeled second molecule and the tagged coupling molecule bind to the binding (here, non-neutralizing) antibodies in the test sample, if present. Labeled first molecules that are not blocked by the functional antibodies 950 (here, nAbs) will bind to the tagged target (here, ACE2-biotin) and be detectable using methods well-known in the art for label-detection. The tagged target (ACE2-biotin) will bind to the anti-tag 970 (here, Streptavidin) on the single test region of the detection zone. If binding antibodies (here, non-nAbs) are present in the test sample, they will bridge the tagged coupling molecules and the labeled second molecules. The tagged coupling molecule will bind to the anti-tag (here, Streptavidin) on the single region of the detection zone, and if the binding antibodies bridge the tagged coupling molecules and the labeled second molecules, the presence of such binding antibodies 960 can be detectable using methods well-known in the art for label-detection. If binding antibodies are not present in the test sample, the labeled second molecules will not be captured on the binding antibodies test region. In the example shown in FIG. 9, the first molecules are labeled with red nanoparticles and the second molecules are labeled with blue nanoparticles. FIG. 10 are images of exemplary test readouts 1000 (red), 1010 (purple), and 1020 (blue) obtained using the test kit of FIG. 9. Where there is simultaneous binding of the red and blue labeled molecules, the single test line will appear purple, as indicated in test readout 1010. A red line indicates no functional antibodies or binding antibodies are present in the test sample. A blue line indicates a high titer of functional antibodies and binding antibodies are present in the test sample. Intermediate samples will have different hues that can be, for example, deconvoluted into two color intensities using a reader equipped with the appropriate software.

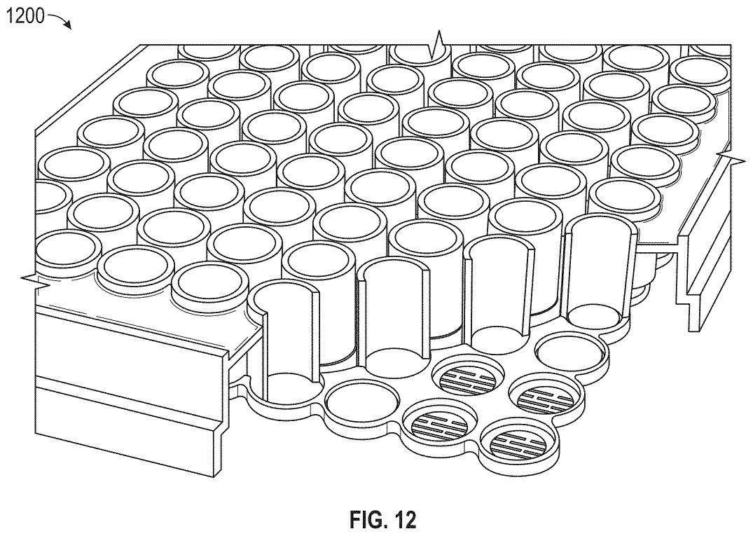

As described above, the immunoassay of a test kit can comprise a lateral flow assay. In some embodiments, the immunoassay of a test kit comprises a vertical flow assay, as shown in FIG. 11. FIG. 11 illustrates an exploded view of a vertical flow assay 1100, which requires less space and can provides a reduced assay time compared to lateral flow assays. Assay 1100 can comprise a bi-color assay and/or a mixed color assay as described above, yet in a vertical configuration. Assay 1100 comprises a card frame 1110, which can comprise any suitable size and shape, and a backing label 1135, which can comprise two openings that align with sample receiving portions of the card frame. Assay 1100 can comprise or more conjugate release pads, one or more active membranes 1120, and one or more absorbent pads 1130 sandwiched between card frame 1110 and backing label 1135. Conjugate release pads can hold and preserve the detection reagents, or conjugate. In some embodiments, when an assay (chase) buffer is added to the sample well, the dried components on the strip interact with plasma or serum from whole blood. As the test sample flows through the conjugate release pad, the conjugate is released into the sample and binds with the functional and/or binding antibodies, if present. Active membranes 1120 can comprise one or more anti-tags bound thereto, and/or any other suitable components, for example, to capture a tag of a tagged target and/or tagged coupling molecule. A well of a vertical flow assay can comprise one or more test regions and optionally a control region. Absorbent pads 830 can absorb excess test samples. In some embodiments, the vertical flow assay is a multi-well vertical flow assay (e.g., a 96-well vertical flow assay 1200 as shown in FIG. 12). The multi-channel vertical flow assay can comprise any suitable number of wells (e.g., at least 4, at least 8, at least 12, at least 16, at least 32, at least 64, at least 96) allows for mass screening/surveillance with multiple tests capable of being performed at the same time. In some embodiments, each well of the multi-channel vertical flow assay can comprise one or more test regions and/or a control region. In some embodiments, each well of the multi-channel vertical flow assay can comprise a sample receiving portion, a conjugate release pad, an active membrane, and an absorbent pad.

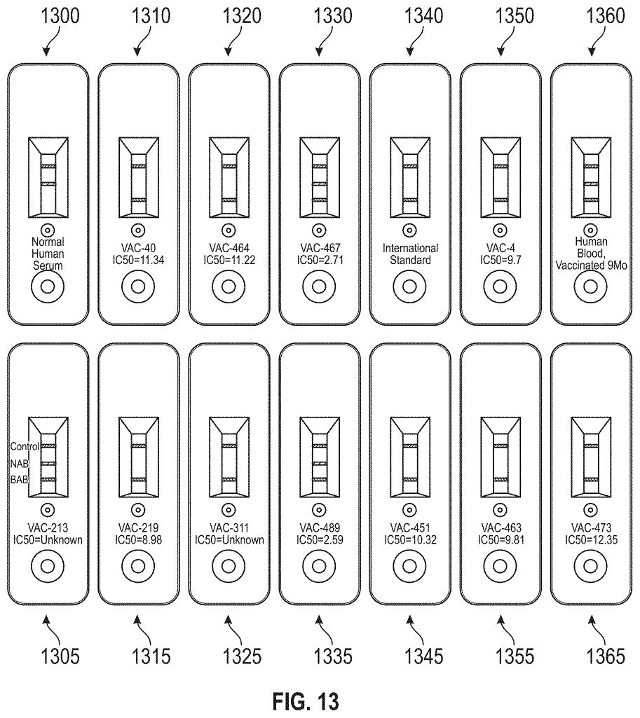

FIG. 13 are images of test results obtained using a test kit of the disclosure, according to an embodiment. The lateral flow assay shown can be used to detect any suitable functional antibodies and binding antibodies. In the examples shown, the lateral flow assays are used to detect neutralizing (functional) and non-neutralizing (binding) antibodies. The test strips were run using several samples, including some samples with known IC50 (Inhibitory Concentration at 50%) determined in live virus focus reduction neutralization test (FRNT). A first line, here the top line, or region on each test strip is a control line that changes color (e.g., from blue to red) when the test is run properly. The first test line or region (shown here as NAB line) is a functional antibodies test region (here, nAb test region). In the example shown, a more intense line is indicative of little to no nAb being present in a sample. No line corresponds to a high concentration of nAbs. The second test line or region (shown here as BAB line or non-neutralizing antibody test region) is a binding antibodies (or general immunity) test region. A more intense line indicates more bAb is present in a sample. No line means no bAb was detected in a sample.

In some embodiments, qualitative interpretation is contemplated wherein the presence of lines at only the control and NAB test regions indicates no immunity, the presence of lines at only the control and BAB test regions indicates strong immunity; and the presence of all three lines (control, NAB, BAB) indicates moderate immunity.

The sample used to run test strip 1300 is normal human serum, without vaccination and without earlier infection. Here, the normal human serum is from an individual that has not been vaccinated for the prevention and/or reduction of illness from a SARS-COV-2 infection, and that has not been infected with COVID-19 disease.

The sample used to run test strip 1310 is a vaccinated individual (subject number 40), with an IC50 value of 11.34. It is contemplated that the IC50 value can be based on the number of times a test sample/serum is diluted to get to 50% inhibition. The test results show no immunity. The sample used to run test strip 1320 is a vaccinated individual (subject number 464), with an IC50 value of 11.22. The test results show strong neutralizing power and high levels of nAbs. The sample used to run test strip 1330 is a vaccinated individual (subject number 467) with an IC50 value of 2.71. The test results show weak to medium nAb levels and bAb levels. The sample used to run test strip 1340 represents the International Standard. The sample used to run test strip 1350 is a vaccinated individual (subject number 4), with an IC50 value of 9.7. The sample used to run test strip 1360 is a sample from a human vaccinated 9 months earlier. The sample used to run test strip 1305 is a vaccinated individual (subject number 213), with an unknown IC50 value. The sample used to run test strip 1315 is a vaccinated individual (subject number 219), with an IC50 value of 8.98. The sample used to run test strip 1325 is a vaccinated individual (subject number 311), with an unknown IC50 value. The sample used to run test strip 1335 is a vaccinated individual (subject number 489), with an IC50 value of 2.59. The sample used to run test strip 1345 is a vaccinated individual (subject number 451), with an IC50 value of 10.32. The sample used to run test strip 1355 is a vaccinated individual (subject number 463), with an IC50 value of 9.81. The sample used to run test strip 1365 is a vaccinated individual (subject number 473), with an IC50 value of 12.35. The benefits of detecting and measuring both functional and binding antibodies can be seen, for example, when viewing test strips 1300 and 1360. While not much difference can be seen on the first test (T1) line, the immune response differences in the samples can clearly be seen and/or measured by the differences in the second test (T2) line.

In some embodiments, the binding antibodies test region can comprise an anti-tag of a first tag/anti-tag pair that is bound to the binding antibodies test region. A corresponding tag of the first pair can be bound to a coupling molecule, which can comprise a non-essential portion of the viral-ACE2 binding protein (e.g., an RBD-less Spike protein, an RBM-less Spike protein). Another molecule, which similarly to the coupling molecule, comprises the non-essential part of a viral-ACE2 binding protein (e.g., another RBD-less Spike protein, another RBM-less Spike protein) can be coupled to a label. A binding antibody (here, the non-neutralizing antibody) of a test sample, if present, binds to each of the labeled molecule comprising the non-essential part of the given protein (here, Spike protein) and the tagged coupling molecule (which can also comprise the non-essential part of the given protein) to form a complex of the binding antibody (here, non-neutralizing antibody), the labeled molecule comprising the non-essential part, and the tagged coupling molecule (an “A-B-C complex”), and the binding antibodies test line can capture the A-B-C complex via the first tag/anti-tag pair.

The functional test region (here, neutralizing antibodies (nAb) test region) can comprise a target (e.g., ACE2 or a fragment thereof). In some embodiments, ACE2 can be bound directly to the nAb test region via covalent bonding. In some embodiments, ACE2 can bind to the nAb test region via a second tag/anti-tag pair. In some embodiments, ACE2 or a fragment thereof is bound to biotin (tag), and streptavidin (anti-tag) is bound to the functional test region.

In some embodiments, the conjugate pad of the immunoassay can comprise a mixture of (a) a first molecule coupled to a first label, the first molecule comprising essential part of the viral-ACE2 binding protein (e.g., RBD or RBM region of a SARS-CoV-2 Spike protein), (b) a coupling molecule bound to a tag, the coupling molecule comprising non-essential part of the viral-ACE2 binding protein (e.g., RBD-less or RBM-less Spike protein), (c) a second molecule coupled to a second label, the second molecule comprising the non-essential part of a viral-ACE2 binding protein (similarly to the coupling molecule), and/or (d) a target bound to a second tag (e.g., ACE2-biotin).

In the examples shown, a higher intensity on the binding antibodies test line corresponds to higher levels of bAbs associated with the test sample, and a higher intensity on the functional antibodies test line corresponds to lower levels of nAbs (or no nAb) in the test sample.

In certain embodiments, the level of functional antibodies (here nAbs) in the test-specimen is reported as falling within a range of pre-determined values. In certain embodiments, the level of binding antibodies (here, non-nAbs) in the test-specimen is reported as falling within a range of pre-determined values.

In some aspects, measuring nAb (or other fAb) levels, measuring non-nAb (or other bAb) levels, determining a ratio of nAbs (or other fAbs) to non-nAbs (or other bAbs), determining a ratio of nAbs (or other fAbs) to total antibodies, determining a ratio of non-nAbs (or other bAbs) to nAbs (or other fAbs), determining a ratio of non-nAbs (or other bAbs) to total antibodies, and/or determining any useful quantitative relation between fAb levels and bAb levels can comprise using an densitometer or other reader or electronic device and/or system as further described below.

While some examples herein relates to a test kits, test kit components, assays, and method for detecting and measuring nAbs and non-nAbs in a bio sample in a single test, it should be appreciated that the molecules, test kits, test kit components, assays, and methods disclosed herein can be used to detect and/or measure any different first and second antibodies, for example, functional and binding antibodies, in a single test as further described in detail herein. As noted above, binding antibodies bind to a protein without affecting its primary or essential function, while functional antibodies affect the essential or primary function of a protein upon binding. For example, it should be appreciated that the assays, test kits, methods and molecules described herein can be suitable for detecting and measuring other functional and binding antibodies, and determining an IC50 value or other value associated with an immune response quality score based on a ratio of functional antibodies to binding antibodies, binding antibodies to functional antibodies, functional antibodies to total antibodies, and/or binding antibodies to total antibodies. Such determination can also be based on, for example, known correlations between known ratios and, for example, known IC50 values.

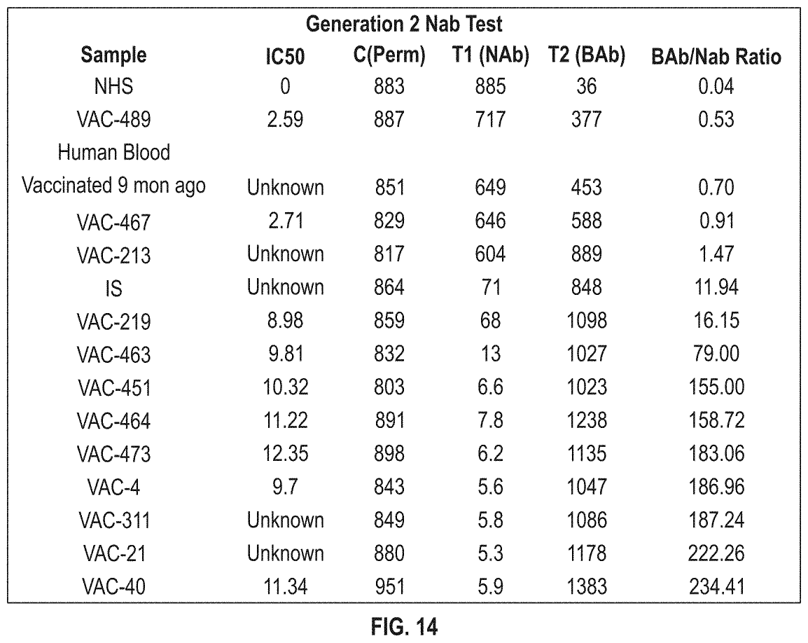

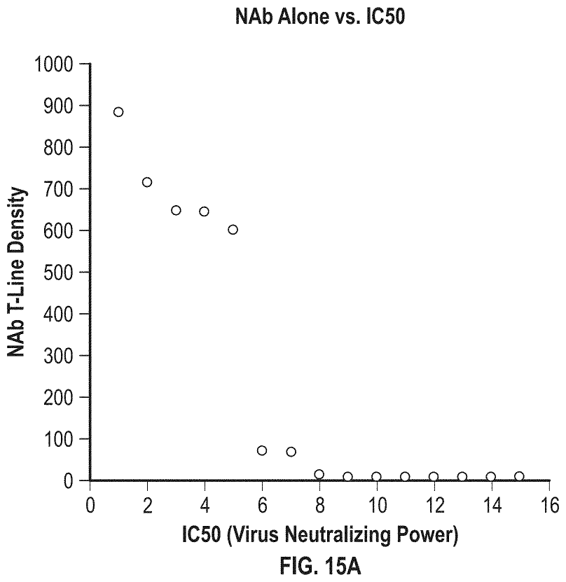

FIG. 14 is a compilation of numeric data obtained from the test results of FIG. 13 using a lateral flow densitometer. The Sample column indicates the sample name, the IC50 column indicates the known IC50 value, if any, or if unknown, indicates “unknown”. The “C (Perm)” column represents a quality (e.g., color intensity, degree of darkness, the optical density) of the control line (C line value), The “T1 (NAb)” column represents a quality (e.g., color intensity, degree of darkness, the optical density) at the functional test line (here, the nAb test line). The quality at the functional test line is referred to as the functional antibodies line value. The “T2 (BAb)” column represents a quality (e.g., color intensity, degree of darkness, the optical density) at the binding antibodies test line (binding antibodies line value). The “BAb/NAb Ratio” column represents a ratio of the binding antibodies line value to the functional antibodies line value, or binding antibodies line value divided by functional antibodies line value.

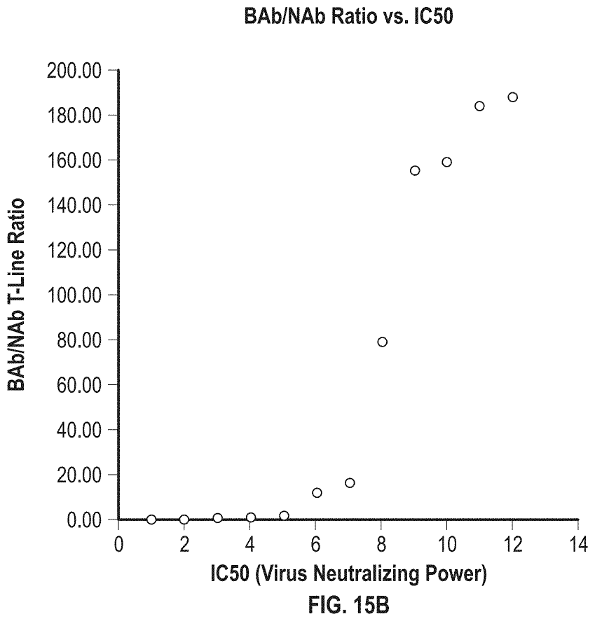

FIG. 15A illustrates a graph of the data from FIG. 14 using nAb alone and IC50. FIG. 15B illustrates a graph of the data from FIG. 14 using bAb/nAb ratio and IC50. FIG. 15B shows that a higher binding antibodies/functional antibodies (here nAb) ratio corresponds to a higher virus neutralizing power. In the examples shown, the bAb line (T2 line) always has a non-0 value (i.e., always has some color) where the sample is from an individual that has been vaccinated or previously infected (here, with COVID19). The functional antibodies test line (here, nAb test line) provides little to no differentiation (not distinguishable by the eye) between samples with higher IC50 (greater than 8), as shown in FIG. 15A. As shown in FIG. 15B, the The bAb/nabs ratios allows for fine differentiation between higher (e.g., >8) IC50 samples. For certain IC50 values, functional (here, nAbs) alone may not provide reliable measurement of immunity. The ratio or other quantitative relation between the functional and binding antibodies allows direct measurement of an immune response quality score. In FIG. 15B, we discovered that the correlation between the bAb and nAb unexpectedly showed to be correlated with IC 50 values. More specifically, the higher the bAb/nAb ratio number, the more efficient the immune response/the more a person is protected. Such a correlation allows us to predict IC50 and/or a quality of immune response based on the ratio or other quantitative relation between binding and functional antibodies rather than ordering an expensive test to determine IC50 value. Thus, the test kits, assays, molecules and methods described herein can be used to calculate a neutralizing power of a test sample. It is contemplated that these concepts can be applied to any disease, including viral diseases, bacterial diseases, cancers, and/or any disease there is or will be a vaccine for.

As discussed above, in some embodiments, a method can comprise using an electronic device to detect and/or quantify functional and binding antibodies present in a sample. In an aspect of the disclosure, a system is provided, comprising at least one measuring device or reader (e.g., densitometer, sensors of a densitometer), configured to measure (a) a first value associated with a first test region of an assay (e.g., a color intensity), and (b) a second value associated with a second test line of the assay (e.g., a color intensity), and a platform comprising at least one hardware processor, and one or more software modules that are configured to, when executed by the at least one hardware processor, receive the first and second values from the at least one measuring device or reader, determine a quantitative relation between the first and second values (e.g., a ratio of a value associated with a color intensity at a first test region and a value associated with a color intensity at a second test region), and calculate an immune response quality score (e.g., an estimated IC50 value) based on the quantitative relation (e.g., ratio). In some aspects, calculating the score can comprise comparing the ratio to data associated with known ratios and associated IC50 values. Such data can be stored in one or more databases as described below. The database(s) may store and maintain any amount and type of data including but not limited to the presence or absence of functional antibodies, the presence or absence of binding antibodies, relative level of functional antibodies and/or binding antibodies, presence or absence of control or test line colors (including that expressed as density units), color intensity for the control line (including that expressed as density units), color intensity (or other characteristic) for one or more test lines, ratio values associated with functional and binding antibodies and associated IC50 values, interpretations of the test results, estimated antibody titers, sample metadata, and/or other sample data such as patient demographic or genomic data, or patient vaccination and/or infection data. In some aspects, the first and second test regions are provided in any test kit or assay described above.

In some embodiments, a system is provided, comprising at least one user system (e.g., a mobile phone, computer, laptop, tablet, or other electronic device) having one or more sensors configured to obtain sensor data associated with a first test region of an assay and a second test region of an assay, and a platform comprising at least one hardware processor, and one or more software modules that are configured to, when executed by the at least one hardware processor, (a) receive the sensor data associated with the first and second test regions from the user system, (b) determine a first value associated with a first test region of an assay (e.g., a quantitative value associated with color intensity), and (c) determine a second value associated with a second test region of the assay (e.g., a quantitative value associated with color intensity), (d) determine a quantitative relation between the first and second values (e.g., a ratio of a value associated with a color intensity at a first test region and a value associated with a color intensity at a second test region), and (e) calculate an immune response quality score (e.g., an estimated IC50 value) based on the quantitative relation (e.g., ratio). In some embodiments, the one or more software modules are further configured to, when executed by the at least one hardware processor, to send an alert or notification to a user system (e.g., the same user system having the one or more sensors, or a different user system or external system) associated with the immune response quality value/score and/or a test sample run on the assay from which the value/score was calculated. In some embodiments, the alert or notification can be associated with available appointment times (e.g., for a visit with a healthcare worker, for a vaccination). In some embodiments, the application can further be configured to receive data from the user system (e.g., a selection of an appointment date and time, health data associated with a user).

In some aspects, calculating the score can comprise comparing the ratio to data associated with known ratios and associated IC50 values. Such data can be stored in one or more databases as described herein. In some aspects, the first and second test regions are provided in any test kit or assay described above.

In an aspect of the disclosure, a system is provided, comprising at least one sensor configured to obtain sensor data associated with a first test region of an assay, and sensor data associated with a second test region of an assay (e.g., image sensor configured to obtain image data, which can be part of an assay reader or other device), a processing system coupled with the at least one sensor and configured to communicate the sensor data associated with the first test region and the sensor data associated with the second test region, and a platform, comprising or coupled to one or more databases (e.g., storing data associated with one or more of a quantitative relation between amounts of functional and binding antibodies in various samples, data associated with IC50 values of known samples, data associated with a correlation between a quantitative relation between amounts of functional and binding antibodies in various samples and IC values, data associated with an immune response quality scores (e.g., grades, categories, and/or numerical values), sample data (e.g., age, vaccination data, infection data, health data, gender data associated with an individual from whom the sample was obtained), and/or any other suitable data), and an application coupled with the database(s) and configured to receive the sensor data, and determine an IC50 value or other immune response quality value/score associated with an individual's ability to recognize and defend themselves against a virus, bacteria, or other invader. In some embodiments, determining the immune response quality value/score can comprise calculating a quantitative relation (e.g., ratio) between data associated with the first test region and data associated with the second test region, and comparing the quantitative relation with data stored in the one or more databases (e.g., data associated with a correlation between a quantitative relation between values associated with amounts of functional and binding antibodies in various samples and IC values). In some embodiments, the application can further be configured to send an alert or notification to a user system associated with the immune response quality value/score. In some embodiments, the alert or notification can be associated with available appointment times (e.g., for a visit with a healthcare worker, for a vaccination). In some embodiments, the application can further be configured to receive data from the user system (e.g., a selection of an appointment date and time, health data associated with a user).

System Overview

1.1. Infrastructure

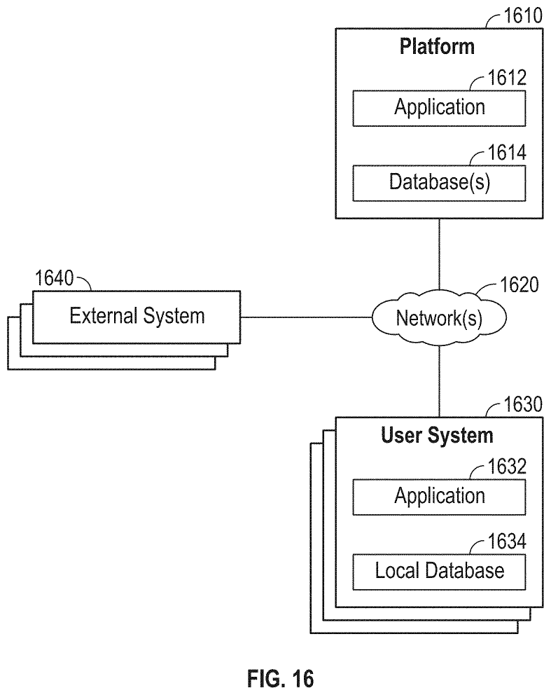

FIG. 16 illustrates an example infrastructure in which one or more of the disclosed processes may be implemented, according to an embodiment. The infrastructure may comprise a platform 1610 (e.g., one or more servers) which hosts and/or executes one or more of the various functions, processes, methods, and/or software modules described herein. Platform 1610 may comprise dedicated servers, or may instead comprise cloud instances, which utilize shared resources of one or more servers. These servers or cloud instances may be collocated and/or geographically distributed. Platform 1610 may also comprise or be communicatively connected to a server application 1612 and/or one or more databases 1614. In addition, platform 1610 may be communicatively connected to one or more user systems 1630 via one or more networks 1620, or may be entirely implemented on the loopback (e.g., localhost) interface. Platform 1610 may also be communicatively connected to one or more external systems 1640 (e.g., other platforms, websites, etc.) via one or more networks 1620.

Network(s) 1620 may comprise the Internet, and platform 1610 may communicate with user system(s) 1630 (e.g., electronic devices, mobile device, assay reader) through the Internet using standard transmission protocols, such as HyperText Transfer Protocol (HTTP), HTTP Secure (HTTPS), File Transfer Protocol (FTP), FTP Secure (FTPS), Secure Shell FTP (SFTP), and the like, as well as proprietary protocols. While platform 1610 is illustrated as being connected to various systems through a single set of network(s) 1620, it should be understood that platform 1610 may be connected to the various systems via different sets of one or more networks. For example, platform 1610 may be connected to a subset of user systems 1630 and/or external systems 1640 via the Internet, but may be connected to one or more other user systems 1630 and/or external systems 1640 via an intranet. Furthermore, while only a few user systems 1630 and external systems 1640, one server application 1612, and one set of database(s) 1614 are illustrated, it should be understood that the infrastructure may comprise any number of user systems, external systems, server applications, and databases. In addition, communication between any of these systems, for example, platform 1610, user systems 1630, and/or external system 1640, may be entirely implemented on the loopback (e.g., localhost) interface.

User system(s) 1630 may comprise any type or types of computing devices capable of wired and/or wireless communication, including without limitation, desktop computers, laptop computers, tablet computers, smart phones or other mobile phones, servers, game consoles, televisions, set-top boxes, electronic kiosks, point-of-sale terminals, and/or the like. Each user system 1630 may comprise or be communicatively connected to a client application 1632 and/or one or more local databases 1634. While user system 1630 and platform 1610 are shown here as separate devices connected by a network 1620. User system 1630 may comprise an application 1632 that may comprise one portion of a distributed cloud-based system that integrates with platform 1610, for example, using a multi-tasking OS (e.g., Linux) and local only (localhost) network addresses.

Platform 1610 may comprise web servers which host one or more websites and/or web services. In embodiments in which a website is provided, the website may comprise a graphical user interface, including, for example, one or more screens (e.g., webpages) generated in HyperText Markup Language (HTML) or other language. Platform 1610 transmits or serves one or more screens of the graphical user interface in response to requests from user system(s) 1630. In some embodiments, these screens may be served in the form of a wizard, in which case two or more screens may be served in a sequential manner, and one or more of the sequential screens may depend on an interaction of the user or user system 1630 with one or more preceding screens. The requests to platform 1610 and the responses from platform 1610, including the screens of the graphical user interface, may both be communicated through network(s) 1620, which may include the Internet, or may be entirely implemented on the loopback (e.g., localhost) interface, using standard communication protocols (e.g., HTTP, HTTPS, etc.). These screens (e.g., webpages) may comprise a combination of content and elements, such as text, images, videos, animations, references (e.g., hyperlinks), frames, inputs (e.g., textboxes, text areas, checkboxes, radio buttons, drop-down menus, buttons, forms, etc.), scripts (e.g., JavaScript), and the like, including elements comprising or derived from data stored in one or more databases (e.g., database(s) 1614) that are locally and/or remotely accessible to platform 1610. Platform 1610 may also respond to other requests from user system(s) 1630.

Platform 1610 may comprise, be communicatively coupled with, or otherwise have access to one or more database(s) 1614. For example, platform 1610 may comprise one or more database servers which manage one or more databases 1614. Server application 1612 executing on platform 1610 and/or client application 1632 executing on user system 1630 may submit data (e.g., user data, form data, etc.) to be stored in database(s) 1614, and/or request access to data stored in database(s) 1614. Any suitable database may be utilized, including without limitation MySQL™, Oracle™, IBM™, Microsoft SQL™, Access™ PostgreSQL™, MongoDB™, and the like, including cloud-based databases and proprietary databases. Data may be sent to platform 1610, for instance, using the well-known POST, GET, and PUT request supported by HTTP, via FTP, proprietary protocols, requests using data encryption via SSL (HTTPS requests), and/or the like. This data, as well as other requests, may be handled, for example, by server-side web technology, such as a servlet or other software module (e.g., comprised in server application 1612), executed by platform 1610.

In embodiments in which a web service is provided, platform 1610 may receive requests from external system(s) 1640, and provide responses in eXtensible Markup Language (XML), JavaScript Object Notation (JSON), and/or any other suitable or desired format. In such embodiments, platform 1610 may provide an application programming interface (API) which defines the manner in which user system(s) 1630 and/or external system(s) 1640 may interact with the web service. Thus, user system(s) 1630 and/or external system(s) 1640 (which may themselves be servers), can define their own user interfaces, and rely on the web service to implement or otherwise provide the backend processes, methods, functionality, storage, and/or the like, described herein. For example, in such an embodiment, a client application 1632, executing on one or more user system(s) 1630, may interact with a server application 1612 executing on platform 1610 to execute one or more or a portion of one or more of the various functions, processes, methods, and/or software modules described herein. In an embodiment, client application 1632 may utilize a local database 1634 for storing data locally on user system 1630.

Client application 1632 may be “thin,” in which case processing is primarily carried out server-side by server application 1612 on platform 1610. A basic example of a thin client application 1632 is a browser application, which simply requests, receives, and renders webpages at user system(s) 1630, while server application 1612 on platform 1610 is responsible for generating the webpages and managing database functions. Alternatively, the client application may be “thick,” in which case processing is primarily carried out client-side by user system(s) 1630. It should be understood that client application 1632 may perform an amount of processing, relative to server application 1612 on platform 1610, at any point along this spectrum between “thin” and “thick,” depending on the design goals of the particular implementation. In any case, the software described herein, which may wholly reside on either platform 1610 (e.g., in which case server application 1612 performs all processing) or user system(s) 1630 (e.g., in which case client application 1632 performs all processing) or be distributed between platform 1610 and user system(s) 130 (e.g., in which case server application 1612 and client application 1632 both perform processing), can comprise one or more executable software modules comprising instructions that implement one or more of the processes, methods, or functions described herein.

While platform 1610, user systems 1630, and external systems 1640 are shown as separate devices communicatively coupled by network 1620, each of the devices shown as platform 1610, user systems 1630, and external systems 1640 may be implemented on one or more devices, and/or one or more of platform 1610, user systems 1630, and external systems 1640 may be implemented on a single device.

1.2. Example Processing Device

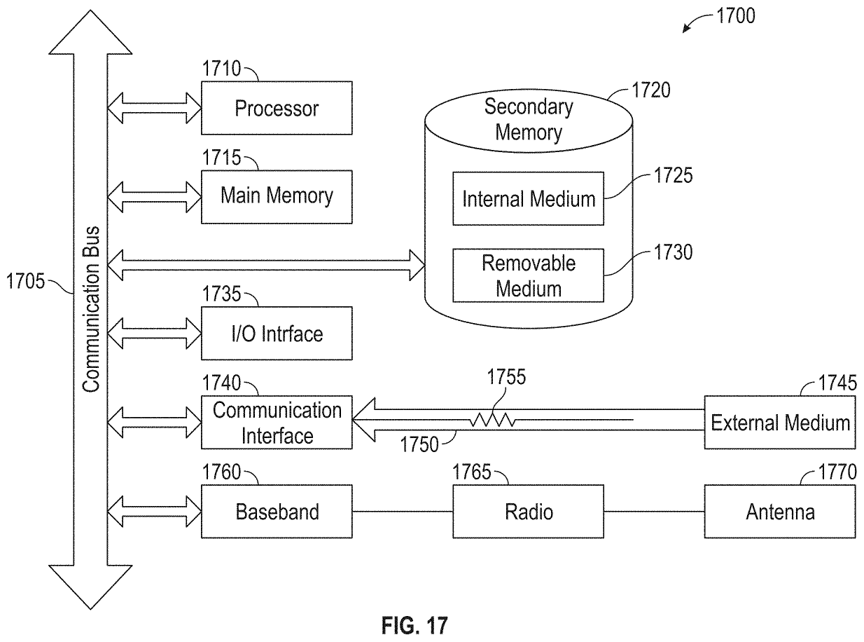

FIG. 17 is a block diagram illustrating an example wired or wireless system 1700 that may be used in connection with various embodiments described herein. For example, system 1700 may be used as or in conjunction with one or more of the functions, processes, or methods (e.g., to store and/or execute the software) described herein, and may represent components of platform 1610, user system(s) 1630, external system(s) 1640, and/or other processing devices described herein. System 1700 can be a server or any conventional personal computer, or any other processor-enabled device that is capable of wired or wireless data communication. Other computer systems and/or architectures may be also used, as will be clear to those skilled in the art.

System 1700 preferably includes one or more processors 1710. Processor(s) 1710 may comprise a central processing unit (CPU). Additional processors may be provided, such as a graphics processing unit (GPU), an auxiliary processor to manage input/output, an auxiliary processor to perform floating-point mathematical operations, a special-purpose microprocessor having an architecture suitable for fast execution of signal-processing algorithms (e.g., digital-signal processor), a slave processor subordinate to the main processing system (e.g., back-end processor), an additional microprocessor or controller for dual or multiple processor systems, and/or a coprocessor. Such auxiliary processors may be discrete processors or may be integrated with processor 1710. Examples of processors which may be used with system 1700 include, without limitation, any of the processors (e.g., Pentium™, Core i7™, Xeon™, etc.) available from Intel Corporation of Santa Clara, Calif., any of the processors available from Advanced Micro Devices, Incorporated (AMD) of Santa Clara, Calif., any of the processors (e.g., A series, M series, etc.) available from Apple Inc. of Cupertino, any of the processors (e.g., Exynos™) available from Samsung Electronics Co., Ltd., of Seoul, South Korea, any of the processors available from NXP Semiconductors N.V. of Eindhoven, Netherlands, and/or the like.

Processor 1710 is preferably connected to a communication bus 1705. Communication bus 1705 may include a data channel for facilitating information transfer between storage and other peripheral components of system 1700. Furthermore, communication bus 1705 may provide a set of signals used for communication with processor 1710, including a data bus, address bus, and/or control bus (not shown). Communication bus 1705 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/S-100, and/or the like.

System 1700 preferably includes a main memory 1715 and may also include a secondary memory 1720. Main memory 1715 provides storage of instructions and data for programs executing on processor 1710, such as any of the software discussed herein. It should be understood that programs stored in the memory and executed by processor 1710 may be written and/or compiled according to any suitable language, including without limitation C/C++, Java, JavaScript, Perl, Visual Basic, .NET, and the like. Main memory 1715 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

Secondary memory 1720 is a non-transitory computer-readable medium having computer-executable code (e.g., any of the software disclosed herein) and/or other data stored thereon. The computer software or data stored on secondary memory 1720 is read into main memory 1715 for execution by processor 1710. Secondary memory 1720 may include, for example, semiconductor-based memory, such as programmable read-only memory (PROM), erasable programmable read-only memory (EPROM), electrically erasable read-only memory (EEPROM), and flash memory (block-oriented memory similar to EEPROM).

Secondary memory 1720 may optionally include an internal medium 1725 and/or a removable medium 1730. Removable medium 1730 is read from and/or written to in any well-known manner. Removable storage medium 1730 may be, for example, a magnetic tape drive, a compact disc (CD) drive, a digital versatile disc (DVD) drive, other optical drive, a flash memory drive, and/or the like.

In alternative embodiments, secondary memory 1720 may include other similar means for allowing computer programs or other data or instructions to be loaded into system 1700. Such means may include, for example, a communication interface 1740, which allows software and data to be transferred from external storage medium 1745 to system 1700. Examples of external storage medium 1745 include an external hard disk drive, an external optical drive, an external magneto-optical drive, and/or the like.

As mentioned above, system 1700 may include a communication interface 1740. Communication interface 1740 allows software and data to be transferred between system 1700 and external devices (e.g. printers), networks, or other information sources. For example, computer software or executable code may be transferred to system 1700 from a network server (e.g., platform 1610) via communication interface 1740. Examples of communication interface 1740 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 1700 with a network (e.g., network(s) 1620) or another computing device. Communication interface 1740 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via communication interface 1740 are generally in the form of electrical communication signals 1755. These signals 1755 may be provided to communication interface 1740 via a communication channel 1750. In an embodiment, communication channel 1750 may be a wired or wireless network (e.g., network(s) 1620), or any variety of other communication links. Communication channel 1750 carries signals 1755 and can be implemented using a variety of wired or wireless communication means including wire or cable, fiber optics, conventional phone line, cellular phone link, wireless data communication link, radio frequency (“RF”) link, or infrared link, just to name a few.

Computer-executable code (e.g., computer programs, such as the disclosed software) is stored in main memory 1715 and/or secondary memory 1720. Computer-executable code can also be received via communication interface 1740 and stored in main memory 1715 and/or secondary memory 220. Such computer programs, when executed, enable system 1700 to perform the various functions of the disclosed embodiments as described elsewhere herein.

In this description, the term “computer-readable medium” is used to refer to any non-transitory computer-readable storage media used to provide computer-executable code and/or other data to or within system 1700. Examples of such media include main memory 1715, secondary memory 1720 (including internal memory 1725, removable medium 1730, and external storage medium 1745), and any peripheral device communicatively coupled with communication interface 1740 (including a network information server or other network device). These non-transitory computer-readable media are means for providing software and/or other data to system 1700.

In an embodiment that is implemented using software, the software may be stored on a computer-readable medium and loaded into system 1700 by way of removable medium 1730, I/O interface 1735, or communication interface 1740. In such an embodiment, the software is loaded into system 1700 in the form of electrical communication signals 1755. The software, when executed by processor 1710, preferably causes processor 1710 to perform one or more of the processes and functions described elsewhere herein.

In an embodiment, I/O interface 1735 provides an interface between one or more components of system 1700 and one or more input and/or output devices. Example input devices include, without limitation, sensors, keyboards, touch screens or other touch-sensitive devices, cameras, biometric sensing devices, computer mice, trackballs, pen-based pointing devices, and/or the like. Examples of output devices include, without limitation, other processing devices, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), and/or the like. In some cases, an input and output device may be combined, such as in the case of a touch panel display (e.g., in a smartphone, tablet, or other mobile device).

System 1700 may also include optional wireless communication components that facilitate wireless communication over a voice network and/or a data network (e.g., in the case of user system 1630). The wireless communication components comprise an antenna system 1770, a radio system 1765, and a baseband system 1760. In system 1700, radio frequency (RF) signals are transmitted and received over the air by antenna system 1770 under the management of radio system 1765.

In an embodiment, antenna system 1770 may comprise one or more antennae and one or more multiplexors (not shown) that perform a switching function to provide antenna system 1770 with transmit and receive signal paths. In the receive path, received RF signals can be coupled from a multiplexor to a low noise amplifier (not shown) that amplifies the received RF signal and sends the amplified signal to radio system 1765.

In an alternative embodiment, radio system 1765 may comprise one or more radios that are configured to communicate over various frequencies. In an embodiment, radio system 1765 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 1765 to baseband system 1760.

If the received signal contains audio information, then baseband system 1760 decodes the signal and converts it to an analog signal. Then the signal is amplified and sent to a speaker. Baseband system 1760 also receives analog audio signals from a microphone. These analog audio signals are converted to digital signals and encoded by baseband system 1760. Baseband system 1760 also encodes the digital signals for transmission and generates a baseband transmit audio signal that is routed to the modulator portion of radio system 1765. The modulator mixes the baseband transmit audio signal with an RF carrier signal, generating an RF transmit signal that is routed to antenna system 1770 and may pass through a power amplifier (not shown). The power amplifier amplifies the RF transmit signal and routes it to antenna system 1770, where the signal is switched to the antenna port for transmission.

Baseband system 1760 is also communicatively coupled with processor(s) 1710. Processor(s) 1710 may have access to data storage areas 1715 and 1720. Processor(s) 1710 are preferably configured to execute instructions (i.e., computer programs, such as the disclosed software) that can be stored in main memory 1715 or secondary memory 1720. Computer programs can also be received from baseband processor 1760 and stored in main memory 1710 or in secondary memory 1720, or executed upon receipt. Such computer programs, when executed, can enable system 1700 to perform the various functions of the disclosed embodiments.

1. Process Overview

Embodiments of processes for obtaining a immune response quality score will now be further described. It should be understood that the described processes may be embodied in one or more software modules that are executed by one or more hardware processors (e.g., processor 1710), for example, as a software application (e.g., server application 1612, client application 1632, and/or a distributed application comprising both server application 1612 and client application 1632), which may be executed wholly by processor(s) of platform 110, wholly by processor(s) of user system(s) 1630, or may be distributed across platform 1610 and user system(s) 1630, such that some portions or modules of the software application are executed by platform 1610 and other portions or modules of the software application are executed by user system(s) 1630. The described processes may be implemented as instructions represented in source code, object code, and/or machine code. These instructions may be executed directly by hardware processor(s) 1710, or alternatively, may be executed by a virtual machine operating between the object code and hardware processor(s) 1710. In addition, the disclosed software may be built upon or interfaced with one or more existing systems.

Alternatively, the described processes may be implemented as a hardware component (e.g., general-purpose processor, integrated circuit (IC), application-specific integrated circuit (ASIC), digital signal processor (DSP), field-programmable gate array (FPGA) or other programmable logic device, discrete gate or transistor logic, etc.), combination of hardware components, or combination of hardware and software components. To clearly illustrate the interchangeability of hardware and software, various illustrative components, blocks, modules, circuits, and steps are described herein generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a component, block, module, circuit, or step is for ease of description. Specific functions or steps can be moved from one component, block, module, circuit, or step to another without departing from the invention.

Furthermore, while the processes, described herein, are illustrated with a certain arrangement and ordering of subprocesses, each process may be implemented with fewer, more, or different subprocesses and a different arrangement and/or ordering of subprocesses. In addition, it should be understood that any subprocess, which does not depend on the completion of another subprocess, may be executed before, after, or in parallel with that other independent subprocess, even if the subprocesses are described or illustrated in a particular order.

In an aspect of the disclosure, a system is provided, comprising: (a) one or more sensors; (b) one or more processors communicatively coupled to the one or more sensors; (c) one or more databases in communication with the one or more processors and configured to store data associated with ratio values associated with functional and binding antibodies and associated IC50 values; and (d) a memory storing software instructions that, when executed by the one or more processors, cause the one or more processors to: (1) obtain, from the one or more sensors, first sensor data associated with a first test line of an assay (e.g., a color intensity) and second sensor data associated with a second test line of an assay, (2) determine a first intensity value associated with the first sensor data, (3) determine a second intensity value associated with the second sensor data, (4) determine a quantitative relation value (e.g., a ratio) associated with the first and second intensity values, and (5) determine an immune response quality value/score based at least in part on the quantitative relation value. In some embodiments, determining an immune response quality value/score comprises comparing the quantitative relation value with data stored in the one or more databases (e.g., data associated with a correlation between a ratio of binding antibodies to functioning antibodies in various samples and associated IC values, or any other suitable data).

Non-Limiting Embodiments.

Embodiment 1. A test kit for detection of a first antibody and a second antibody in a test specimen, comprising: a first molecule comprising a first portion of a protein, wherein the first antibody has a first affinity to bind to the first portion; and a second molecule comprising a second portion of the protein different from the first portion, wherein the second antibody has a second affinity to bind to the second portion.

Embodiment 2. The test kit of embodiment 1, further comprising a target molecule for the first molecule (e.g., a target molecule the first molecule has an affinity to bind to), and an immunoassay having a detection zone, the detection zone comprising at least one test location, and wherein the at least one test location comprises a first anti-tag.

Embodiment 3. The test kit of any of embodiments 1-2, wherein the at least one test location comprises a second anti-tag.

Embodiment 4. The test kit of any of embodiments 1-3, wherein the at least one test location comprises two test locations.

Embodiment 5. The test kit of any of embodiments 1-4, wherein the target molecule is bound to a first tag, wherein the first molecule is coupled to a first label, wherein the second molecule is bound to a second label, and wherein a coupling molecule is bound to a second tag, the coupling molecule comprising the second portion of the protein.

Embodiment 6. The test kit of any of embodiments 1-5, wherein the first tag is the same as the second tag, and wherein the detection zone comprises a single test location.

Embodiment 7. The test kit of any of embodiments 1-6, wherein the first tag and the first anti-tag comprise a first tag/anti-tag pair, and wherein the second tag and the second anti-tag comprise a second tag/anti-tag pair.

Embodiment 8. The test kit of any of embodiments 1-7, wherein the immunoassay further comprises a sample receiving portion and a conjugate release pad, wherein the sample receiving portion comprises at least one of a sample pad and a sample filter.

Embodiment 9. The test kit of any of embodiments 1-8, wherein the detection zone comprises a nitrocellulose membrane.

Embodiment 10. The test kit of any of embodiments 1-9, wherein the protein is a viral-ACE2-binding protein, wherein the first portion comprises an ACE2-binding motif of a receptor binding domain (RBD), wherein the first antibody is a neutralizing antibody (NAb), wherein the second portion lacks the ACE2-binding motif of the RBD, wherein the second antibody is a non-neutralizing antibody (nNAb), and wherein the target molecule for the first molecule comprises ACE2 or a functional fragment thereof.

Embodiment 11. The test kit of any of embodiments 1-10, wherein the first antibody is a functional antibody, wherein the second antibody is a binding antibody, wherein the first portion of the protein comprises an essential portion of the protein, and wherein the second portion comprises a non-essential portion of the protein.

Embodiment 12. A test kit for detection of a functional and binding in a test specimen, comprising: a first molecule comprising an essential portion of a protein, a second molecule comprising a non-essential portion of the protein separate from the essential portion of the protein, and a target molecule for the essential portion of the protein (e.g., a target molecule the essential portion of the protein has an affinity to bind to).

Embodiment 13. The test kit of embodiment 12, wherein the first antibody has a first affinity to bind to the essential portion, and the second antibody has an affinity to bind to the non-essential portion.

Embodiment 14. The test kit of any of embodiments 12-13, further comprising an immunoassay having a detection zone, the detection zone comprising at least one test location, and wherein the at least one test location comprises a first anti-tag.

Embodiment 15. The test kit of any of embodiments 12-14 wherein the at least one test location comprises a second anti-tag.

Embodiment 16. The test kit of any of embodiments 12-15, wherein the at least one test location comprises two test locations.

Embodiment 17. The test kit of any of embodiments 12-16, wherein the target molecule is bound to a first tag, wherein the first molecule is coupled to a first label, wherein the second molecule is bound to a second label, and further comprising a coupling molecule is coupled to a second tag, the coupling molecule comprising the second portion of the protein.

Embodiment 18. The test kit of any of embodiments 12-17, wherein the first tag is the same as the second tag, and wherein the detection zone comprises a single test location.

Embodiment 19. The test kit of any of embodiments 12-18, wherein the first tag and the first anti-tag comprise a first tag/anti-tag pair, and wherein the second tag and the second anti-tag comprise a second tag/anti-tag pair.

Embodiment 20. The test kit of any of embodiments 12-19, wherein the immunoassay further comprises a sample receiving portion and a conjugate release pad, wherein the sample receiving portion comprises at least one of a sample pad and a sample filter.

Embodiment 21. The test kit of any of embodiments 12-20, wherein the detection zone comprises a nitrocellulose membrane.

Embodiment 22. The test kit of any of embodiments 12-21, wherein the protein is a viral-ACE2-binding protein, wherein the essential portion comprises an ACE2-binding motif of a receptor binding domain (RBD), wherein the functional antibody is a neutralizing antibody (NAb), wherein the non-essential portion lacks the ACE2-binding motif of the RBD, wherein the binding antibody is a non-neutralizing antibody (nNAb), and wherein the target molecule for the first molecule comprises ACE2 or a functional fragment thereof.

Embodiment 23. The test kit of any of embodiments 12-22, wherein the at least one test location comprises a functional antibodies test location comprising the first anti-tag, and a binding antibodies test location comprising a second anti-tag different from the first anti-tag.

Embodiment 24. The test kit of any of embodiments 12-23, wherein the functional antibodies are at least one of neutralizing antibodies, blocking antibodies, and enhancing antibodies.

Embodiment 25. The test kit of any of embodiments 12-24, wherein the protein is an enzyme, wherein the essential portion is important for its catalytic activity, and wherein the functional antibodies bind to the essential portion and deactivate or enhance enzymatic activity.

Embodiment 26. The test kit of any of embodiments 12-25, wherein the protein is a cytokine, wherein the essential portion is a cytokine receptor, and wherein the functional antibodies bind to the essential portion and prevent efficient cytokine-drive intracellular signaling.

Embodiment 27. The test kit of any of embodiments 12-26, wherein the protein is a receptor, wherein the essential portion is a portion essential for binding with its ligand, and wherein the functional antibodies bind to the essential portion prevent efficient receptor-driven intracellular signaling.

Embodiment 28. The test kit of any of embodiments 12-27, wherein the protein plays a scaffold function, wherein the essential portion is essential for binding with a second protein that is important for adequate function of a multi-subunit protein complex, and wherein the functional antibodies bind to the essential portion and prevent the binding with the second protein.

Embodiment 29. The test kit of any of embodiments 12-28, the essential portion and the non-essential portion comprise engineered portions of the protein.

Embodiment 30. The test kit of any of embodiments 12-29, wherein the immunoassay is a vertical flow assay.

Embodiment 31. The test kit of any of embodiments 12-30, wherein the vertical flow assay is a multi-well vertical flow assay.

Embodiment 32. The test kit of any of embodiments 12-30, wherein the immunoassay is a lateral flow assay.

Embodiment 33. A method for detection of first and second antibodies in a test specimen, comprising: obtaining the test specimen from a subject; transferring the test specimen to a sample receiving portion of an assay of a test kit, wherein the test kit further comprises: a first molecule comprising a first portion of a protein, wherein the first antibodies have a first affinity to bind to the first portion; a second molecule comprising a second portion of the protein different from the first portion, wherein the second antibodies have a second affinity to bind to the second portion; and a target molecule for the first molecule (e.g., target molecule the first molecule has an affinity to bind to); and reading the results from the assay.

Embodiment 34. The method of embodiment 33, wherein the assay comprises a detection zone at least one test location, and wherein the at least one test location comprises a first anti-tag.

Embodiment 35. The method of any of embodiments 33-34, wherein the protein is a viral-ACE2-binding protein, wherein the first portion comprises an ACE2-binding domain of the viral-ACE2-binding protein, wherein the second portion lacks the ACE2-binding domain of the viral-ACE2-binding protein, and wherein the target molecule is ACE2 of a functional fragment thereof.

Embodiment 36. The method of any of embodiments 33-35, wherein the detection zone comprises a single test location comprising a first anti-tag, wherein the target molecule is bound to a first tag, wherein the first molecule is coupled to a first label, wherein the second molecule is coupled to a second label, and further comprising a coupling molecule coupled to a second tag, the coupling molecule comprising the second portion of the protein.

Embodiment 37. The method of any of embodiments 33-36, further comprising determining a ratio of the first antibodies to the second antibodies using a color deconvolution algorithm.

Embodiment 38. The method of any of embodiments 33-37. wherein the test specimen is obtained from a patient that is at least one of known to be recovering from COVID19 disease, known to have been vaccinated for SARS-CoV-2, and suspected to be recovering from COVID19 disease.

Embodiment 39. The method of any of embodiments 33-38, wherein the target molecule is bound to biotin, and wherein the first anti-tag comprises streptavidin.

Embodiment 40. The method of any of embodiments 33-39, wherein the detection zone comprises a first antibodies test location comprising the first anti-tag, a second antibodies test location comprising a second anti-tag, wherein the target molecule is bound to a first tag, wherein the first molecule is coupled to a first label, wherein the second molecule is bound to a second label, and further comprising a coupling molecule coupled to a second tag, the coupling molecule comprising the second portion of the protein.

Embodiment 41. The method of any of embodiments 33-40, wherein each of the first label and the second label is selected from a nanoparticle, bead, latex bead, aptamer, oligonucleotide, a quantum dot, and a combination thereof.

Embodiment 42. The method of any of embodiments 33-41, wherein the first molecule is coupled to a first nanoparticle, and wherein the coupling molecule is coupled to a second nanoparticle.

Embodiment 43. The method of any of embodiments 33-42, wherein the first molecule is coupled to a gold nanoshell (GNS), and wherein the coupling molecule is coupled to a gold nanosphere (GNP).

Embodiment 44. A protein variant including an amino acid sequence selected from the group consisting of SEQ ID NOs: 8, 9, 11, and 12.

The following literature is incorporated herein in their entireties:

Neutralizing antibodies to therapeutic enzymes: considerations for testing, prevention and treatment. Wang J, Lozier J, Johnson G, Kirshner S, Verthelyi D, Pariser A, Shores E, Rosenberg A. Nat Biotechnol. 2008 August; 26(8):901-8. doi: 10.1038/nbt.1484. PMID: 18688246.

The impact of the immune system on the safety and efficiency of enzyme replacement therapy in lysosomal storage disorders. Broomfield A, Jones S A, Hughes S M, Bigger B W. J Inherit Metab Dis. 2016 July; 39(4):499-512. doi: 10.10071s10545-016-9917-1. Epub 2016 Feb. 16. PMID: 26883220.

Development of an enzymatic assay for the detection of neutralizing antibodies against therapeutic angiotensin-converting enzyme 2 (ACE2). Liao K, Sikkema D, Wang C, Lee T N. J Immunol Methods. 2013 Mar. 29; 389(1-2):52-60. doi: 10.1016/j.jim.2012.12.010. Epub 2013 Jan. 5. PMID: 23298658.

Enzyme therapy for Fabry disease: neutralizing antibodies toward agalsidase alpha and beta. Linthorst G E, Hollak C E, Donker-Koopman W E, Strijland A, Aerts J M. Kidney Int. 2004 October; 66(4):1589-95. doi: 10.1111/j.1523-1755.2004.00924.x. PMID: 15458455.

Rescuing AAV gene transfer from neutralizing antibodies with an IgG-degrading enzyme. Elmore Z C, Oh D K, Simon K E, Fanous M M, Asokan A. JCI Insight. 2020 Sep. 17; 5(19):e139881. doi: 10.1172/jci.insight.139881. PMID: 32941184; PMCID: PMC7566709.

Interleukin-6 neutralizing antibody attenuates the hypersecretion of airway mucus via inducing the nuclear translocation of Nrf2 in chronic obstructive pulmonary disease. Wei Y Y, Zhang D W, Ye J J, Lan Q X, Ji S, Sun L, Li F, Fei G H. Biomed Pharmacother. 2022 August; 152:113244. doi: 10.1016/j.biopha.2022.113244. Epub 2022 Jun. 7. PMID: 35687911.

Thus, specific examples of test kits, test kit components and methods for detecting and measuring antibodies have been described. The above description of the disclosed embodiments is provided to enable any person skilled in the art to make or use the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

As used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Reference throughout this specification to “an embodiment” or “an implementation” means that a particular feature, structure, or characteristic described in connection with the embodiment is included in at least one embodiment or implementation. Thus, appearances of the phrases “in an embodiment” in various places throughout this specification are not necessarily all referring to the same embodiment or a single exclusive embodiment. Furthermore, the particular features, structures, or characteristics described herein may be combined in any suitable manner in one or more embodiments or one or more implementations.

The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Certain numerical values and ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

All structural and functional equivalents to the components of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

SEQUENCE LISTING
SEQ ID NO 1: An exemplary RBD domain of a SARS-COV-2 spike protein:
RVQPTESIVRFPNITNLCPFGEVFNATRFASVYAWNRKRISNCVADYSVLYNSASFSTFKCYGVSPT
KLNDLCFTNVYADSFVIRGDEVRQIAPGQTGKIADYNYKLPDDFTGCVIAWNSNNLDSKVGGNY
NYLYRLFRKSNLKPFERDISTEIYQAGSTPCNGVEGFNCYFPLQSYGFPTNGVGYQPYRVVVLSFE
LLHAPATVCGPKKSTNLVKNKCVNFNFNGLTGTGVLTESNKKFLPFQQFGRDIADTTDAVRDPQT
LEILDITPCS.
SEQ ID NO 2: an exemplary sequence for the RBD domain, which
corresponds to amino acids 319-541 of SARS-COV-2 spike:
QRVQPTESIVRFPNITNLCPFGEVFNATRFASVYAWNRKRISNCVADYSVLYNSASFSTFKCYGVSP
TKLNDLCFTNVYADSFVIRGDEVRQIAPGQTGKIADYNYKLPDDFTGCVIAWNSNNLDSKVGGNY
NYLYRLFRKSNLKPFERDISTEIYQAGSTPCNGVEGFNCYFPLQSYGFQPTNGVGYQPYRVVVLSF
ELLHAPATVCGPKKSTNLVKNKCVNF.
SEQ ID NO 3: an exemplary sequence used herein for the ACE2 domain,
which corresponds to amino acids 18-615 of the full-length human
ACE2:
QSTIEEQAKTFLDKFNHEAEDLFYQSSLASWNYNTNITEENVQNMNNAGDKWSAFLKEQSTLAQ
MYPLQEIQNLTVKLQLQALQQNGSSVLSEDKSKRLNTILNTMSTIYSTGKVCNPDNPQECLLLEPG
LNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMWGRF
WTNLYSLTVPFGQKPNIDVTDAMVDQAWDAQRIFKEAEKFFVSVGLPNMTQGFWENSMLTDPG
NVQKAVCHPTAWDLGKGDFRILMCTKVTMDDFLTAHHEMGHIQYDMAYAAQPFLLRNGANEG
FHEAVGEIMSLSAATPKHLKSIGLLSPDFQEDNETEINFLLKQALTIVGTLPFTYMLEKWRWMVFK
GEIPKDQWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDQNKNSFVGWSTDWSPYAD.
SEQ ID NO 4: cDNA sequence encoding full-length spike trimer
protein. Parts encoding signal peptide, T4-phage fibritin
trimerization domain and 6xHis tag are indicated between
parentheses (“( )” - round brackets), brackets (“[ ]” -
square brackets), and braces (“{ }” - curly brackets),
respectively. Protein translation initiation and stop
codons are shown in bold. Cloning restriction sites,
5′ BamHI and 3′ XhoI are underlined in italic:
GGATCCGCCACC(ATGTTCGTGTTTCTGGTGCTGCTGCCTCTGGTGTCCAGC)CAGTGTGTGAAC
CTGACCACAAGAACCCAGCTGCCTCCTGCGTACACAAACAGCTTCACCCGGGGAGTGTACTAC
CCCGATAAGGTGTTCCGTAGCTCCGTGCTGCACTCTACACAGGACCTGTTCCTCCCCTTTTTCT
CTAACGTGACTTGGTTCCACGCCATCCACGTGAGTGGCACCAACGGCACCAAGAGATTCGAC
AATCCTGTTCTGCCCTTCAACGACGGCGTGTACTTCGCCAGCACAGAGAAGAGCAACATCATC
AGAGGATGGATCTTCGGCACCACTCTCGATAGCAAGACCCAGTCTCTGCTGATCGTCAACAAT
GCCACCAATGTGGTGATCAAGGTTTGTGAATTCCAGTTCTGCAACGACCCTTTTCTGGGCGTTT
ACTATCACAAGAATAACAAGTCCTGGATGGAAAGCGAGTTTCGGGTGTATTCTTCTGCCAACA
ACTGTACCTTCGAGTACGTGTCTCAACCTTTTCTGATGGACCTGGAAGGCAAGCAGGGCAACT
TCAAAAACCTGAGAGAATTCGTGTTCAAGAACATTGATGGCTACTTCAAAATCTACAGCAAGC
ACACACCAATCAACCTGGTGAGAGACCTGCCTCAGGGCTTCAGCGCCCTGGAACCCCTGGTG
GACCTGCCTATTGGCATTAACATCACCAGATTCCAGACCCTGTTGGCTCTGCACAGAAGCTAC
CTGACACCTGGCGACTCCAGCAGCGGATGGACCGCCGGCGCCGCTGCTTACTACGTGGGCTAC
CTGCAGCCTAGGACATTCCTACTGAAGTACAATGAGAACGGCACCATCACCGATGCCGTGGA
TTGCGCCCTGGACCCTCTGAGTGAAACCAAGTGTACCCTGAAATCGTTCACTGTCGAGAAGGG
CATCTACCAGACCAGCAACTTCAGAGTGCAGCCTACAGAGAGCGCTTCGGTGGCGTGTCTGTG
ATCACCCCGGGTACCAACACCAGCAACCAGGTGGCTGTTCTTTACCAGGGAGTGAACTGCACC
GAAGTGCCCGTGGCCATTCACGCAGACCAGCTGACCCCCACCTGGCGGGTGTACTCAACAGG
CAGCAATGTGTTCCAGACTCGGGCCGGATGTCTGATCGGAGCTGAACACGTGAACAATAGCT
ACGAGTGCGACATCCCCATCGGCGCTGGCATCTGCGCCTCTTACCAGACCCAGACCAACTCCC
CAGGATCTGCCTCTTCCGTGGCCTCTCAGAGCATCATCGCCTACACCATGAGCCTGGGAGCCG
AGAATAGCGTGGCTTACAGCAACAACTCCATCGCGATCCCTACAAACTTCACCATCTCTGTGA
CCACCGAGATCCTGCCGGTTTCTATGACCAAGACCAGCGTTGATTGCACCATGTACATCTGCG
GCGATTCCACAGAGTGCAGCAACCTGCTGCTGCAATACGGCAGCTTTTGCACCCAGCTCAACA
GAGCCCTGACCGGCATCGCAGTTGAGCAGGATAAGAACACACAGGAGGTTTTCGCCCAAGTG
AAACAAATCTACAAGACCCCTCCTATCAAGGACTTCGGCGGGTTTAATTTCAGCCAAATCCTG
CCTGATCCTTCTAAACCCAGCGCGGGTTCTCCCATCGAGGACCTGCTGTTCAACAAGGTAACA
CTCGCTGACGCCGGCTTCATCAAGCAGTATGGCGACTGCCTGGGCGATATCGCTGCCAGAGAC
CTGATCTGCGCCCAGAAATTCAACGGCCTGACGGTGCTGCCTCCTCTGCTGACCGACGAGATG
ATCGCCCAGTATACCTCTGCCCTCCTGGCCGGAACAATCACCAGCGGCTGGACCTTCGGCGCC
GGACCTGCCCTCCAGATTCCCTTCCCTATGCAGATGGCCTACCGGTTCAACGGAATCGGCGTC
ACCCAAAACGTGCTGTACGAGAACCAGAAACTGATCGCTAATCAGTTCAACAGCGCCATCGG
AAAGATCCAGGACAGCTTGAGTAGCACACCAAGCGCCCTGGGCAAGCTGCAGGATGTGGTTA
ATCAGAACGCCCAGGCCCTGAACACCCTGGTTAAGCAGTTAAGCTCTAACTTTGGCGCCATCA
GCTCCGTGCTGAATGACATCCTCAGCAGACTGGACCCTCCTGAGGCCGAGGTGCAGATCGACC
GGCTGATTACAGGGCGGCTGCAAAGCCTGCAGACATACGTGACACAGCAACTAATCCGGGCT
GCCGAGATCAGAGCCTCCGCCAACCTGGCCGCCACAAAGATGTCCGAATGCGTGCTCGGACA
GAGCAAGCGAGTGGACTTCTGCGGAAAGGGCTACCACCTGATGAGCTTCCCACAGTCCGCCC
CCCACGGCGTCGTGTTCCTGCACGTGACATATGTGCCTGCACAGGAAAAAAACTTCACAACAG
CCCCTGCCATCTGCCACGACGGCAAGGCCCACTTCCCCAGAGAGGGCGTGTTCGTGTCCAACG
GAACACACTGGTTCGTGACCCAAAGAAACTTCTACGAGCCTCAGATCATCACCACCGATAATA
CCTTCGTCAGCGGCAACTGCGACGTGGTGATCGGCATCGTGAACAACACGGTGTACGACCCGT
TGCAACCAGAGTTGGATAGCTTTAAGGAAGAGCTGGACAAGTACTTCAAGAATCACACCTCC
CCTGACGTGGACCTGGGCGACATCTCCGGCATCAACGCCAGCGTGGTGAACATCCAGAAGGA
AATCGATAGACTTAACGAAGTGGCCAAGAACCTGAACGAGAGCCTGATCGACCTTCAAGAGC
TGGGCAAATACGAGCAGTACATCAAGTGGCCT[GGCAGCGGTTACATCCCTGAAGCCCCTAGA
GACGGCCAGGCCTATGTGCGGAAAGATGGCGAATGGGTCCTGCTGAGCACGTTTCTG]GGA{C
ATCATCATCATCATCAC}TAATGACTCGAG.
SEQ ID NO 5: .cDNA sequence encoding “Loop-less” spike trimer
protein. Parts encoding signal peptide, T4-phage fibritin
trimerization domain and 6xHis tag are indicated between
parentheses (“( )” - round brackets), brackets (“[ ]” -
square brackets), and braces (“{ }” - curly brackets),
respectively. Protein translation initiation and stop codons
are shown in bold. Cloning restriction sites, 5′ BamHI and
3′ XhoI are underlined in italic:
GGATCCGCCACC(ATGTTCGTGTTTCTGGTGCTGCTGCCTCTGGTGTCCAGC)CAGTGTGTGAAC
CTGACCACAAGAACCCAGCTGCCTCCTGCGTACACAAACAGCTTCACCCGGGGAGTGTACTAC
CCCGATAAGGTGTTCCGTAGCTCCGTGCTGCACTCTACACAGGACCTGTTCCTCCCCTTTTTCT
CTAACGTGACTTGGTTCCACGCCATCCACGTGAGTGGCACCAACGGCACCAAGAGATTCGAC
AATCCTGTTCTGCCCTTCAACGACGGCGTGTACTTCGCCAGCACAGAGAAGAGCAACATCATC
AGAGGATGGATCTTCGGCACCACTCTCGATAGCAAGACCCAGTCTCTGCTGATCGTCAACAAT
GCCACCAATGTGGTGATCAAGGTTTGTGAATTCCAGTTCTGCAACGACCCTTTTCTGGGCGTTT
ACTATCACAAGAATAACAAGTCCTGGATGGAAAGCGAGTTTCGGGTGTATTCTTCTGCCAACA
ACTGTACCTTCGAGTACGTGTCTCAACCTTTTCTGATGGACCTGGAAGGCAAGCAGGGCAACT
TCAAAAACCTGAGAGAATTCGTGTTCAAGAACATTGATGGCTACTTCAAAATCTACAGCAAGC
ACACACCAATCAACCTGGTGAGAGACCTGCCTCAGGGCTTCAGCGCCCTGGAACCCCTGGTG
GACCTGCCTATTGGCATTAACATCACCAGATTCCAGACCCTGTTGGCTCTGCACAGAAGCTAC
CTGACACCTGGCGACTCCAGCAGCGGATGGACCGCCGGCGCCGCTGCTTACTACGTGGGCTAC
CTGCAGCCTAGGACATTCCTACTGAAGTACAATGAGAACGGCACCATCACCGATGCCGTGGA
TTGCGCCCTGGACCCTCTGAGTGAAACCAAGTGTACCCTGAAATCGTTCACTGTCGAGAAGGG
CATCTACCAGACCAGCAACTTCAGAGTGCAGCCTACAGAGAGCATCGTGCGGTTCCCTAACAT
CACAAATCTGTGTCCTTTCGGCGAGGTGTTCAACGCCACAAGATTTGCCTCAGTGTACGCTTG
GAATAGGAAGAGAATCTCCAACTGTGTGGCCGACTATAGCGTTCTGTACAACAGCGCGAGCT
TCAGCACCTTCAAGTGCTACGGCGTTAGCCCTACCAAGCTGAACGACCTGTGCTTCACCAACG
TGTACGCCGACAGCTTCGTCATCAGAGGAGATGAGGTGAGACAGATCGCCCCTGGCCAGACA
GGCAAAATCGCCGACTACAACTACAAGCTGCCTGACGACTTCACTGGCTGCGTGATCGCCTGG
AACAGCAACAACCTGGACAGCAAGGTGGGCGGCACAAATGGAGTGGGCTACCAGCCCTACCG
CGTGGTGGTGCTGAGCTTCGAGCTGCTGCACGCCCCTGCTACCGTGTGCGGCCCAAAAAAGTC
TACCAACCTGGTGAAGAATAAGTGCGTGAACTTTAACTTCAACGGCCTGACAGGAACCGGCG
TCCTGACCGAAAGCAACAAAAAGTTCCTGCCATTCCAACAGTTTGGCAGAGATATCGCTGACA
CCACCGACGCCGTGCGGGACCCTCAGACCCTGGAAATCCTGGACATAACACCCTGTAGCTTCG
GTGGCGTGTCTGTGATCACCCCGGGTACCAACACCAGCAACCAGGTGGCTGTTCTTTACCAGG
GAGTGAACTGCACCGAAGTGCCCGTGGCCATTCACGCAGACCAGCTGACCCCCACCTGGCGG
GTGTACTCAACAGGCAGCAATGTGTTCCAGACTCGGGCCGGATGTCTGATCGGAGCTGAACA
CGTGAACAATAGCTACGAGTGCGACATCCCCATCGGCGCTGGCATCTGCGCCTCTTACCAGAC
CCAGACCAACTCCCCAGGATCTGCCTCTTCCGTGGCCTCTCAGAGCATCATCGCCTACACCAT
GAGCCTGGGAGCCGAGAATAGCGTGGCTTACAGCAACAACTCCATCGCGATCCCTACAAACT
TCACCATCTCTGTGACCACCGAGATCCTGCCGGTTTCTATGACCAAGACCAGCGTTGATTGCA
CCATGTACATCTGCGGCGATTCCACAGAGTGCAGCAACCTGCTGCTGCAATACGGCAGCTTTT
GCACCCAGCTCAACAGAGCCCTGACCGGCATCGCAGTTGAGCAGGATAAGAACACACAGGAG
GTTTTCGCCCAAGTGAAACAAATCTACAAGACCCCTCCTATCAAGGACTTCGGCGGGTTTAAT
TTCAGCCAAATCCTGCCTGATCCTTCTAAACCCAGCGCGGGTTCTCCCATCGAGGACCTGCTG
TTCAACAAGGTAACACTCGCTGACGCCGGCTTCATCAAGCAGTATGGCGACTGCCTGGGCGAT
ATCGCTGCCAGAGACCTGATCTGCGCCCAGAAATTCAACGGCCTGACGGTGCTGCCTCCTCTG
CTGACCGACGAGATGATCGCCCAGTATACCTCTGCCCTCCTGGCCGGAACAATCACCAGCGGC
TGGACCTTCGGCGCCGGACCTGCCCTCCAGATTCCCTTCCCTATGCAGATGGCCTACCGGTTC
AACGGAATCGGCGTCACCCAAAACGTGCTGTACGAGAACCAGAAACTGATCGCTAATCAGTT
CAACAGCGCCATCGGAAAGATCCAGGACAGCTTGAGTAGCACACCAAGCGCCCTGGGCAAGC
TGCAGGATGTGGTTAATCAGAACGCCCAGGCCCTGAACACCCTGGTTAAGCAGTTAAGCTCTA
ACTTTGGCGCCATCAGCTCCGTGCTGAATGACATCCTCAGCAGACTGGACCCTCCTGAGGCCG
AGGTGCAGATCGACCGGCTGATTACAGGGCGGCTGCAAAGCCTGCAGACATACGTGACACAG
CAACTAATCCGGGCTGCCGAGATCAGAGCCTCCGCCAACCTGGCCGCCACAAAGATGTCCGA
ATGCGTGCTCGGACAGAGCAAGCGAGTGGACTTCTGCGGAAAGGGCTACCACCTGATGAGCT
TCCCACAGTCCGCCCCCCACGGCGTCGTGTTCCTGCACGTGACATATGTGCCTGCACAGGAAA
AAAACTTCACAACAGCCCCTGCCATCTGCCACGACGGCAAGGCCCACTTCCCCAGAGAGGGC
GTGTTCGTGTCCAACGGAACACACTGGTTCGTGACCCAAAGAAACTTCTACGAGCCTCAGATC
ATCACCACCGATAATACCTTCGTCAGCGGCAACTGCGACGTGGTGATCGGCATCGTGAACAAC
ACGGTGTACGACCCGTTGCAACCAGAGTTGGATAGCTTTAAGGAAGAGCTGGACAAGTACTT
CAAGAATCACACCTCCCCTGACGTGGACCTGGGCGACATCTCCGGCATCAACGCCAGCGTGGT
GAACATCCAGAAGGAAATCGATAGACTTAACGAAGTGGCCAAGAACCTGAACGAGAGCCTG
ATCGACCTTCAAGAGCTGGGCAAATACGAGCAGTACATCAAGTGGCCT[GGCAGCGGTTACAT
CCCTGAAGCCCCTAGAGACGGCCAGGCCTATGTGCGGAAAGATGGCGAATGGGTCCTGCTGA
GCACGTTTCTG]GGA{CATCATCATCATCATCAC}TAATGACTCGAG.
SEQ ID NO 6: cDNA sequence encoding “RBD-less” spike trimer
protein. Parts encoding signal peptide, T4 fibritin
trimerization domain and 6xHis tag are indicated between
parentheses (“( )” - round brackets), brackets (“[ ]” -
square brackets), and braces (“{ }” - curly brackets),
respectively. Protein translation initiation and stop
codons are shown in bold. Cloning restriction sites,
5′ BamHI and 3′ XhoI are underlined in italic:
GGATCCGCCACC(ATGTTCGTGTTTCTGGTGCTGCTGCCTCTGGTGTCCAGC)CAGTGTGTGAAC
CTGACCACAAGAACCCAGCTGCCTCCTGCGTACACAAACAGCTTCACCCGGGGAGTGTACTAC
CCCGATAAGGTGTTCCGTAGCTCCGTGCTGCACTCTACACAGGACCTGTTCCTCCCCTTTTTCT
CTAACGTGACTTGGTTCCACGCCATCCACGTGAGTGGCACCAACGGCACCAAGAGATTCGAC
AATCCTGTTCTGCCCTTCAACGACGGCGTGTACTTCGCCAGCACAGAGAAGAGCAACATCATC
AGAGGATGGATCTTCGGCACCACTCTCGATAGCAAGACCCAGTCTCTGCTGATCGTCAACAAT
GCCACCAATGTGGTGATCAAGGTTTGTGAATTCCAGTTCTGCAACGACCCTTTTCTGGGCGTTT
ACTATCACAAGAATAACAAGTCCTGGATGGAAAGCGAGTTTCGGGTGTATTCTTCTGCCAACA
ACTGTACCTTCGAGTACGTGTCTCAACCTTTTCTGATGGACCTGGAAGGCAAGCAGGGCAACT
TCAAAAACCTGAGAGAATTCGTGTTCAAGAACATTGATGGCTACTTCAAAATCTACAGCAAGC
ACACACCAATCAACCTGGTGAGAGACCTGCCTCAGGGCTTCAGCGCCCTGGAACCCCTGGTG
GACCTGCCTATTGGCATTAACATCACCAGATTCCAGACCCTGTTGGCTCTGCACAGAAGCTAC
CTGACACCTGGCGACTCCAGCAGCGGATGGACCGCCGGCGCCGCTGCTTACTACGTGGGCTAC
CTGCAGCCTAGGACATTCCTACTGAAGTACAATGAGAACGGCACCATCACCGATGCCGTGGA
TTGCGCCCTGGACCCTCTGAGTGAAACCAAGTGTACCCTGAAATCGTTCACTGTCGAGAAGGG
CATCTACCAGACCAGCAACTTCAGAGTGCAGgctAGCTTCGGTGGCGTGTCTGTGATCACCCCG
GGTACCAACACCAGCAACCAGGTGGCTGTTCTTTACCAGGGAGTGAACTGCACCGAAGTGCC
CGTGGCCATTCACGCAGACCAGCTGACCCCCACCTGGCGGGTGTACTCAACAGGCAGCAATG
TGTTCCAGACTCGGGCCGGATGTCTGATCGGAGCTGAACACGTGAACAATAGCTACGAGTGC
GACATCCCCATCGGCGCTGGCATCTGCGCCTCTTACCAGACCCAGACCAACTCCCCAGGATCT
GCCTCTTCCGTGGCCTCTCAGAGCATCATCGCCTACACCATGAGCCTGGGAGCCGAGAATAGC
GTGGCTTACAGCAACAACTCCATCGCGATCCCTACAAACTTCACCATCTCTGTGACCACCGAG
ATCCTGCCGGTTTCTATGACCAAGACCAGCGTTGATTGCACCATGTACATCTGCGGCGATTCC
ACAGAGTGCAGCAACCTGCTGCTGCAATACGGCAGCTTTTGCACCCAGCTCAACAGAGCCCTG
ACCGGCATCGCAGTTGAGCAGGATAAGAACACACAGGAGGTTTTCGCCCAAGTGAAACAAAT
CTACAAGACCCCTCCTATCAAGGACTTCGGCGGGTTTAATTTCAGCCAAATCCTGCCTGATCC
TTCTAAACCCAGCGCGGGTTCTCCCATCGAGGACCTGCTGTTCAACAAGGTAACACTCGCTGA
CGCCGGCTTCATCAAGCAGTATGGCGACTGCCTGGGCGATATCGCTGCCAGAGACCTGATCTG
CGCCCAGAAATTCAACGGCCTGACGGTGCTGCCTCCTCTGCTGACCGACGAGATGATCGCCCA
GTATACCTCTGCCCTCCTGGCCGGAACAATCACCAGCGGCTGGACCTTCGGCGCCGGACCTGC
CCTCCAGATTCCCTTCCCTATGCAGATGGCCTACCGGTTCAACGGAATCGGCGTCACCCAAAA
CGTGCTGTACGAGAACCAGAAACTGATCGCTAATCAGTTCAACAGCGCCATCGGAAAGATCC
AGGACAGCTTGAGTAGCACACCAAGCGCCCTGGGCAAGCTGCAGGATGTGGTTAATCAGAAC
GCCCAGGCCCTGAACACCCTGGTTAAGCAGTTAAGCTCTAACTTTGGCGCCATCAGCTCCGTG
CTGAATGACATCCTCAGCAGACTGGACCCTCCTGAGGCCGAGGTGCAGATCGACCGGCTGATT
ACAGGGCGGCTGCAAAGCCTGCAGACATACGTGACACAGCAACTAATCCGGGCTGCCGAGAT
CAGAGCCTCCGCCAACCTGGCCGCCACAAAGATGTCCGAATGCGTGCTCGGACAGAGCAAGC
GAGTGGACTTCTGCGGAAAGGGCTACCACCTGATGAGCTTCCCACAGTCCGCCCCCCACGGCG
TCGTGTTCCTGCACGTGACATATGTGCCTGCACAGGAAAAAAACTTCACAACAGCCCCTGCCA
TCTGCCACGACGGCAAGGCCCACTTCCCCAGAGAGGGCGTGTTCGTGTCCAACGGAACACAC
TGGTTCGTGACCCAAAGAAACTTCTACGAGCCTCAGATCATCACCACCGATAATACCTTCGTC
AGCGGCAACTGCGACGTGGTGATCGGCATCGTGAACAACACGGTGTACGACCCGTTGCAACC
AGAGTTGGATAGCTTTAAGGAAGAGCTGGACAAGTACTTCAAGAATCACACCTCCCCTGACG
TGGACCTGGGCGACATCTCCGGCATCAACGCCAGCGTGGTGAACATCCAGAAGGAAATCGAT
AGACTTAACGAAGTGGCCAAGAACCTGAACGAGAGCCTGATCGACCTTCAAGAGCTGGGCAA
ATACGAGCAGTACATCAAGTGGCCT[GGCAGCGGTTACATCCCTGAAGCCCCTAGAGACGGCC
AGGCCTATGTGCGGAAAGATGGCGAATGGGTCCTGCTGAGCACGTTTCTG]GGA{CATCATCA
TCATCATCAC}TAATGACTCGAG.
SEQ ID NO 7: Protein sequences of the Full-length spike trimer
protein variant. Signal peptide, T4-phage fibritin trimerization
domain, and 6xHs tag are indicated between parentheses (“( )” -
round brackets), brackets (“[ ]” - square brackets), and braces
(“{ }” - curly brackets), respectively:
(MFVFLVLLPLVSS)QCVNLTTRTQLPPAYTNSFTRGVYYPDKVFRSSVLHSTQDLFLPFFSNVTWF
HAIHVSGTNGTKRFDNPVLPFNDGVYFASTEKSNIIRGWIFGTTLDSKTQSLLIVNNATNVVIKVCE
FQFCNDPFLGVYYHKNNKSWMESEFRVYSSANNCTFEYVSQPFLMDLEGKQGNFKNLREFVFKNI
DGYFKIYSKHTPINLVRDLPQGFSALEPLVDLPIGINITRFQTLLALHRSYLTPGDSSSGWTAGAAA
YYVGYLQPRTFLLKYNENGTITDAVDCALDPLSETKCTLKSFTVEKGIYQTSNFRVQPTESIVRFPN
ITNLCPFGEVFNATRFASVYAWNRKRISNCVADYSVLYNSASFSTFKCYGVSPTKLNDLCFTNVYA
DSFVIRGDEVRQIAPGQTGKIADYNYKLPDDFTGCVIAWNSNNLDSKVGGNYNYLYRLFRKSNLK
PFERDISTEIYQAGSTPCNGVEGFNCYFPLQSYGFQPTNGVGYQPYRVVVLSFELLHAPATVCGPK
KSTNLVKNKCVNFNFNGLTGTGVLTESNKKFLPFQQFGRDIADTTDAVRDPQTLEILDITPCSFGG
VSVITPGTNTSNQVAVLYQGVNCTEVPVAIHADQLTPTWRVYSTGSNVFQTRAGCLIGAEHVNNS
YECDIPIGAGICASYQTQTNSPGSASSVASQSIIAYTMSLGAENSVAYSNNSIAIPTNFTISVTTEILPV
SMTKTSVDCTMYICGDSTECSNLLLQYGSFCTQLNRALTGIAVEQDKNTQEVFAQVKQIYKTPPIK
DFGGFNFSQILPDPSKPSAGSPIEDLLFNKVTLADAGFIKQYGDCLGDIAARDLICAQKFNGLTVLPP
LLTDEMIAQYTSALLAGTITSGWTFGAGPALQIPFPMQMAYRFNGIGVTQNVLYENQKLIANQFNS
AIGKIQDSLSSTPSALGKLQDVVNQNAQALNTLVKQLSSNFGAISSVLNDILSRLDPPEAEVQIDRLI
TGRLQSLQTYVTQQLIRAAEIRASANLAATKMSECVLGQSKRVDFCGKGYHLMSFPQSAPHGVVF
LHVTYVPAQEKNFTTAPAICHDGKAHFPREGVFVSNGTHWFVTQRNFYEPQIITTDNTFVSGNCD
VVIGIVNNTVYDPLQPELDSFKEELDKYFKNHTSPDVDLGDISGINASVVNIQKEIDRLNEVAKNLN
ESLIDLQELGKYEQYIKWP[GSGYIPEAPRDGQAYVRKDGEWVLLSTFL]G{HHHHHH}.
SEQ ID NO 8: Protein sequences of “Loop-less” spike trimer protein
variant. Signal peptide, T4-phagefibritin trimerization domain,
and 6xHs tag are indicated parentheses (“( )” - round brackets),
brackets (“[ ]” - square brackets), and braces (“{ }” - curly
brackets), respectively:
(MFVFLVLLPLVSS)QCVNLTTRTQLPPAYTNSFTRGVYYPDKVFRSSVLHSTQDLFLPFFSNVTWF
HAIHVSGTNGTKRFDNPVLPFNDGVYFASTEKSNIIRGWIFGTTLDSKTQSLLIVNNATNVVIKVCE
FQFCNDPFLGVYYHKNNKSWMESEFRVYSSANNCTFEYVSQPFLMDLEGKQGNFKNLREFVFKNI
DGYFKIYSKHTPINLVRDLPQGFSALEPLVDLPIGINITRFQTLLALHRSYLTPGDSSSGWTAGAAA
YYVGYLQPRTFLLKYNENGTITDAVDCALDPLSETKCTLKSFTVEKGIYQTSNFRVQPTESIVRFPN
ITNLCPFGEVFNATRFASVYAWNRKRISNCVADYSVLYNSASFSTFKCYGVSPTKLNDLCFTNVYA
DSFVIRGDEVRQIAPGQTGKIADYNYKLPDDFTGCVIAWNSNNLDSKVGGTNGVGYQPYRVVVLS
FELLHAPATVCGPKKSTNLVKNKCVNFNFNGLTGTGVLTESNKKFLPFQQFGRDIADTTDAVRDP
QTLEILDITPCSFGGVSVITPGTNTSNQVAVLYQGVNCTEVPVAIHADQLTPTWRVYSTGSNVFQT
RAGCLIGAEHVNNSYECDIPIGAGICASYQTQTNSPGSASSVASQSIIAYTMSLGAENSVAYSNNSIA
IPTNFTISVTTEILPVSMTKTSVDCTMYICGDSTECSNLLLQYGSFCTQLNRALTGIAVEQDKNTQE
VFAQVKQIYKTPPIKDFGGFNFSQILPDPSKPSAGSPIEDLLFNKVTLADAGFIKQYGDCLGDIAARD
LICAQKFNGLTVLPPLLTDEMIAQYTSALLAGTITSGWTFGAGPALQIPFPMQMAYRFNGIGVTQN
VLYENQKLIANQFNSAIGKIQDSLSSTPSALGKLQDVVNQNAQALNTLVKQLSSNFGAISSVLNDIL
SRLDPPEAEVQIDRLITGRLQSLQTYVTQQLIRAAEIRASANLAATKMSECVLGQSKRVDFCGKGY
HLMSFPQSAPHGVVFLHVTYVPAQEKNFTTAPAICHDGKAHFPREGVFVSNGTHWFVTQRNFYEP
QKEIDRLNEVAKNLNESLIDLQELGKYEQYIKWP[GSGYIPEAPRDGQAYVRKDGEWVLLSTFL]G
{HHHHHH}.
SEQ ID NO 9: Protein sequences of “RBD-less” spike trimer protein
variant. Signal peptide, T4-phage fibritin trimerization domain,
and 6xHs tag are indicated between parentheses (“( )″ - round
brackets), brackets (“[ ]” - square brackets), and braces
(“{ }” -curly brackets), respectively:
(MFVFLVLLPLVSS)QCVNLTTRTQLPPAYTNSFTRGVYYPDKVFRSSVLHSTQDLFLPFFSNVTWF
HAIHVSGTNGTKRFDNPVLPFNDGVYFASTEKSNIIRGWIFGTTLDSKTQSLLIVNNATNVVIKVCE
FQFCNDPFLGVYYHKNNKSWMESEFRVYSSANNCTFEYVSQPFLMDLEGKQGNFKNLREFVFKNI
DGYFKIYSKHTPINLVRDLPQGFSALEPLVDLPIGINITRFQTLLALHRSYLTPGDSSSGWTAGAAA
YYVGYLQPRTFLLKYNENGTITDAVDCALDPLSETKCTLKSFTVEKGIYQTSNFRVQASFGGVSVI
TPGTNTSNQVAVLYQGVNCTEVPVAIHADQLTPTWRVYSTGSNVFQTRAGCLIGAEHVNNSYEC
DIPIGAGICASYQTQTNSPGSASSVASQSIIAYTMSLGAENSVAYSNNSIAIPTNFTISVTTEILPVSMT
KTSVDCTMYICGDSTECSNLLLQYGSFCTQLNRALTGIAVEQDKNTQEVFAQVKQIYKTPPIKDFG
GFNFSQILPDPSKPSAGSPIEDLLFNKVTLADAGFIKQYGDCLGDIAARDLICAQKFNGLTVLPPLLT
DEMIAQYTSALLAGTITSGWTFGAGPALQIPFPMQMAYRFNGIGVTQNVLYENQKLIANQFNSAIG
KIQDSLSSTPSALGKLQDVVNQNAQALNTLVKQLSSNFGAISSVLNDILSRLDPPEAEVQIDRLITG
RLQSLQTYVTQQLIRAAEIRASANLAATKMSECVLGQSKRVDFCGKGYHLMSFPQSAPHGVVFLH
VTYVPAQEKNFTTAPAICHDGKAHFPREGVFVSNGTHWFVTQRNFYEPQIITTDNTFVSGNCDVVI
GIVNNTVYDPLQPELDSFKEELDKYFKNHTSPDVDLGDISGINASVVNIQKEIDRLNEVAKNLNESL
IDLQELGKYEQYIKWP[GSGYIPEAPRDGQAYVRKDGEWVLLSTFL]G{HHHHHH}.
SEQ ID NO 10: Protein sequences of the Full-length spike monomer
protein variant. Signal peptide and 6xHs tag are indicated between
parentheses (“( )” - round brackets), brackets (“[ ]” - square
brackets), and braces (“{ }” - curly brackets), respectively:
(MFVFLVLLPLVSS)QCVNLTTRTQLPPAYTNSFTRGVYYPDKVFRSSVLHSTQDLFLPFFSNVTWF
HAIHVSGTNGTKRFDNPVLPFNDGVYFASTEKSNIIRGWIFGTTLDSKTQSLLIVNNATNVVIKVCE
FQFCNDPFLGVYYHKNNKSWMESEFRVYSSANNCTFEYVSQPFLMDLEGKQGNFKNLREFVFKNI
DGYFKIYSKHTPINLVRDLPQGFSALEPLVDLPIGINITRFQTLLALHRSYLTPGDSSSGWTAGAAA
YYVGYLQPRTFLLKYNENGTITDAVDCALDPLSETKCTLKSFTVEKGIYQTSNFRVQPTESIVRFPN
ITNLCPFGEVFNATRFASVYAWNRKRISNCVADYSVLYNSASFSTFKCYGVSPTKLNDLCFTNVYA
DSFVIRGDEVRQIAPGQTGKIADYNYKLPDDFTGCVIAWNSNNLDSKVGGNYNYLYRLFRKSNLK
PFERDISTEIYQAGSTPCNGVEGFNCYFPLQSYGFQPTNGVGYQPYRVVVLSFELLHAPATVCGPK
KSTNLVKNKCVNFNFNGLTGTGVLTESNKKFLPFQQFGRDIADTTDAVRDPQTLEILDITPCSFGG
VSVITPGTNTSNQVAVLYQGVNCTEVPVAIHADQLTPTWRVYSTGSNVFQTRAGCLIGAEHVNNS
YECDIPIGAGICASYQTQTNSPGSASSVASQSIIAYTMSLGAENSVAYSNNSIAIPTNFTISVTTEILPV
SMTKTSVDCTMYICGDSTECSNLLLQYGSFCTQLNRALTGIAVEQDKNTQEVFAQVKQIYKTPPIK
DFGGFNFSQILPDPSKPSAGSPIEDLLFNKVTLADAGFIKQYGDCLGDIAARDLICAQKFNGLTVLPP
LLTDEMIAQYTSALLAGTITSGWTFGAGPALQIPFPMQMAYRFNGIGVTQNVLYENQKLIANQFNS
AIGKIQDSLSSTPSALGKLQDVVNQNAQALNTLVKQLSSNFGAISSVLNDILSRLDPPEAEVQIDRLI
TGRLQSLQTYVTQQLIRAAEIRASANLAATKMSECVLGQSKRVDFCGKGYHLMSFPQSAPHGVVF
LHVTYVPAQEKNFTTAPAICHDGKAHFPREGVFVSNGTHWFVTQRNFYEPQIITTDNTFVSGNCD
VVIGIVNNTVYDPLQPELDSFKEELDKYFKNHTSPDVDLGDISGINASVVNIQKEIDRLNEVAKNLN
ESLIDLQELGKYEQYIKWPG{HHHHHH}.
SEQ ID NO 11: Protein sequences of “Loop-less” spike monomer protein
variant. Signal peptide, and 6xHs tag are indicated between
parentheses (“( )” - round brackets), brackets (“[ ]” - square
brackets), and braces (“{ }” - curly brackets), respectively:
(MFVFLVLLPLVSS)QCVNLTTRTQLPPAYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLIVNNATNVVI
KVCEFQFCNDPFLGVYYHKNNKSWMESEFRVYSSANNCTFEYVSQPFLMDLEGKQGNFKNLREF
VFKNIDGYFKIYSKHTPINLVRDLPQGFSALEPLVDLPIGINITRFQTLLALHRSYLTPGDSSSGWTA
GAAAYYVGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLDSKVGGTNGVGYQPY
RVVVLSFELLHAPATVCGPKKSTNLVKNKCVNFNFNGLTGTGVLTESNKKFLPFQQFGRDIADTT
DAVRDPQTLEILDITPCSFGGVSVITPGTNTSNQVAVLYQGVNCTEVPVAIHADQLTPTWRVYSTG
SNVFQTRAGCLIGAEHVNNSYECDIPIGAGICASYQTQTNSPGSASSVASQSIIAYTMSLGAENSVA
YSNNSIAIPTNFTISVTTEILPVSMTKTSVDCTMYICGDSTECSNLLLQYGSFCTQLNRALTGIAVEQ
DKNTQEVFAQVKQIYKTPPIKDFGGFNFSQILPDPSKPSAGSPIEDLLFNKVTLADAGFIKQYGDCL
GDIAARDLICAQKFNGLTVLPPLLTDEMIAQYTSALLAGTITSGWTFGAGPALQIPFPMQMAYRFN
GIGVTQNVLYENQKLIANQFNSAIGKIQDSLSSTPSALGKLQDVVNQNAQALNTLVKQLSSNFGAI
SSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFV
TQRNFYEPQUITTDNTFVSGNCDVVIGIVNNTVYDPLQPELDSFKEELDKYFKNHTSPDVDLGDISGI
NASVVNIQKEIDRLNEVAKNLNESLIDLQELGKYEQYIKWPG{HHHHHH}.
SEQ ID NO 12: Protein sequences of “RBD-less” spike monomer protein
variant. Signal peptide and 6xHs tag are indicated between parentheses
(“( )” - round brackets), brackets (“[ ]” - square brackets), and
braces (“{ }” - curly brackets), respectively:
(MFVFLVLLPLVSS)QCVNLTTRTQLPPAYTNSFTRGVYYPDKVFRSSVLHSTQDLFLPFFSNVTWF
HAIHVSGTNGTKRFDNPVLPFNDGVYFASTEKSNIIRGWIFGTTLDSKTQSLLIVNNATNVVIKVCE
FQFCNDPFLGVYYHKNNKSWMESEFRVYSSANNCTFEYVSQPFLMDLEGKQGNFKNLREFVFKNI
DGYFKIYSKHTPINLVRDLPQGFSALEPLVDLPIGINITRFQTLLALHRSYLTPGDSSSGWTAGAAA
YYVGYLQPRTFLLKYNENGTITDAVDCALDPLSETKCTLKSFTVEKGIYQTSNFRVQASFGGVSVI
TPGTNTSNQVAVLYQGVNCTEVPVAIHADQLTPTWRVYSTGSNVFQTRAGCLIGAEHVNNSYEC
DIPIGAGICASYQTQTNSPGSASSVASQSIIAYTMSLGAENSVAYSNNSIAIPTNFTISVTTEILPVSMT
KTSVDCTMYICGDSTECSNLLLQYGSFCTQLNRALTGIAVEQDKNTQEVFAQVKQIYKTPPIKDFG
GFNFSQILPDPSKPSAGSPIEDLLFNKVTLADAGFIKQYGDCLGDIAARDLICAQKFNGLTVLPPLLT
DEMIAQYTSALLAGTITSGWTFGAGPALQIPFPMQMAYRFNGIGVTQNVLYENQKLIANQFNSAIG
KIQDSLSSTPSALGKLQDVVNQNAQALNTLVKQLSSNFGAISSVLNDILSRLDPPEAEVQIDRLITG
RLQSLQTYVTQQLIRAAEIRASANLAATKMSECVLGQSKRVDFCGKGYHLMSFPQSAPHGVVFLH
VTYVPAQEKNFTTAPAICHDGKAHFPREGVFVSNGTHWFVTQRNFYEPQIITTDNTFVSGNCDVVI
GIVNNTVYDPLQPELDSFKEELDKYFKNHTSPDVDLGDISGINASVVNIQKEIDRLNEVAKNLNESL
IDLQELGKYEQYIKWPG{HHHHHH}.

1. A test kit for detection of functional and binding antibodies in a test specimen, comprising:
a first molecule comprising an essential portion of a protein;
a second molecule comprising a non-essential portion of the protein separate from the essential portion of the protein; and
a target molecule for the essential portion of the protein.

2. The test kit of claim 1, further comprising an immunoassay having a detection zone, the detection zone comprising at least one test location, and wherein the at least one test location comprises a first anti-tag.

3. The test kit of claim 2, wherein the at least one test location comprises a second anti-tag.

4. The test kit of claim 3, wherein the at least one test location comprises two test locations.

5. The test kit of claim 2, wherein the target molecule is bound to a first tag, wherein the first molecule is coupled to a first label, wherein the second molecule is bound to a second label, and further comprising a coupling molecule is coupled to a second tag, the coupling molecule comprising the second portion of the protein.

6. The test kit of claim 5, wherein the first tag is the same as the second tag, and wherein the detection zone comprises a single test location.

7. The test kit of claim 5, wherein the first tag and the first anti-tag comprise a first tag/anti-tag pair, and wherein the second tag and the second anti-tag comprise a second tag/anti-tag pair.

8. The test kit of claim 2, wherein the immunoassay further comprises a sample receiving portion and a conjugate release pad, wherein the sample receiving portion comprises at least one of a sample pad and a sample filter.

9. The test kit of claim 2, wherein the detection zone comprises a nitrocellulose membrane.

10. The test kit of claim 1, wherein the protein is a viral-ACE2-binding protein, wherein the essential portion comprises an ACE2-binding motif of a receptor binding domain (RBD), wherein the functional antibody is a neutralizing antibody (NAb), wherein the non-essential portion lacks the ACE2-binding motif of the RBD, wherein the binding antibody is a non-neutralizing antibody (nNAb), and wherein the target molecule for the first molecule comprises ACE2 or a functional fragment thereof.

11. The test kit of claim 2, wherein the at least one test location comprises a functional antibodies test location comprising the first anti-tag, and a binding antibodies test location comprising a second anti-tag different from the first anti-tag.

12. The test kit of claim 1, wherein the functional antibodies are at least one of neutralizing antibodies, blocking antibodies, and enhancing antibodies.

13. The test kit of claim 1, wherein the protein is an enzyme, wherein the essential portion is important for its catalytic activity, and wherein the functional antibodies bind to the essential portion and deactivate or enhance enzymatic activity.

14. The test kit of claim 1, wherein the protein is a cytokine, wherein the essential portion is a cytokine receptor, and wherein the functional antibodies bind to the essential portion and prevent efficient cytokine-drive intracellular signaling.

15. The test kit of claim 1, wherein the protein is a receptor, wherein the essential portion is a portion essential for binding with its ligand, and wherein the functional antibodies bind to the essential portion prevent efficient receptor-driven intracellular signaling.

16. The test kit of claim 1, wherein the protein plays a scaffold function, wherein the essential portion is essential for binding with a second protein that is important for adequate function of a multi-subunit protein complex, and wherein the functional antibodies bind to the essential portion and prevent the binding with the second protein.

17. A method for determining an immune response quality score of a test specimen from detection of first and second antibodies in a test specimen, comprising:
obtaining the test specimen from a subject;
transferring the test specimen to a sample receiving portion of an assay of a test kit, the test kit further comprising a functional antibodies test line and a binding antibodies test line; and
obtaining the immune response quality score based on a correlation between a binding antibodies test line value and a functional antibodies test line value.

18. The method of claim 17, wherein obtaining the immune response quality score comprises obtaining the binding antibodies test line value and the functional antibodies test line value, and dividing the binding antibodies test line value by the functional antibodies test line value.

19. The method of claim 18, wherein the binding antibodies test line value is associated with a color intensity on the binding antibodies test line after transferring the test specimen to the sample receiving portion of the assay and waiting at least a pre-determined amount of time.

20. The method of claim 19, wherein the functional antibodies test line value is associated with a color intensity on the functional antibodies test line after transferring the test specimen to the sample receiving portion of the assay and waiting at least the pre-determined amount of time.

21. The method of claim 20, wherein each of the binding antibodies test line value and functional antibodies test line values are based on a comparing the color intensity on the functional antibodies test line with a first scorecard and comparing the color intensity on the functional antibodies test line with a second scorecard.

22. The method of claim 20, wherein each of the binding antibodies test line value and functional antibodies test line values are based on a reader that is configured to quantify an intensity of the binding antibodies test line and the functional antibodies test line.
